(19) United States
(12) Patent Application Publication
(10) Pub. No.: US 2026/0315549 A1
(43) Pub. Date: Oct. 15, 2026

(54) COMPOSITIONS AND METHODS FOR TREATING CLOSTRIDIOIDES DIFFICILE INFECTION

(71) Applicant: Vedanta Biosciences, Inc., Cambridge, MA (US)

(72) Inventors: Rajita Menon, Brighton, MA (US); Jason Norman, North Weymouth, MA (US); Bernat Olle, Cambridge, MA (US)

(73) Assignee: Vedanta Biosciences, Inc., Cambridge, MA (US)

(21) Appl. No.: 19/478,851

(22) PCT Filed: Apr. 26, 2024
(86) PCT No.: PCT/US2024/026572

§ 371 Date: Oct. 27, 2025

Related U.S. Application Data
(60) Provisional application No. 63/498,985, filed on Apr. 28, 2023.

(51) Int. Cl. A61K 35/742; A61K 35/00; A61K 35/741; A61K 38/14; A61P 31/04; C12Q 1/06

(52) U.S. Cl. CPC A61K 35/742; A61K 35/741; A61K 38/14; A61P 31/04; C12Q 1/06; A61K 2035/115

(57) ABSTRACT

Provided herein are methods for treating or preventing recurrent Clostridioides difficile infection, comprising bacterial strains belonging to species Clostridium bolteae, Anaerotruncus colihominis, and/or Flavonifractor plautii. Also provided are compositions comprising bacterial mixtures of strains including Clostridium bolteae, Anaerotruncus colihominis, Sellimonas intestinalis, Clostridium symbiostim, Blautia producta, Dorea longicatena, Clostridium innocuum, Flavonifractor plautii, Lachnospiraceae bacterium, and/or Bifidobacterium longum. Also provided are methods of using compositions comprising such bacterial mixtures (e.g., for treating or preventing Clostridium difficile infection).

### RELATED APPLICATION

This application claims the benefit under 35 U.S.C. § 119(e) of U.S. provisional application No. 63/498,985, filed Apr. 28, 2023, the contents of which are incorporated by reference herein in their entirety.

### GOVERNMENT LICENSE RIGHTS

This invention was made with government support under Contract No. 75A50120C00177 awarded by the Biomedical Advanced Research and Development Authority. The government has certain rights in the invention.

### REFERENCE TO AN ELECTRONIC SEQUENCE LISTING

The contents of the electronic sequence listing (P074570033WO00-SEQ-NTJ.xml; Size: 25,041 bytes; and Date of Creation: Apr. 23, 2024) are herein incorporated by reference in their entirety.

### FIELD

The disclosure relates to compositions including two or more bacterial strains, and methods for treating pathogenic infections, such as a Clostridioides difficile (formerly Clostridium difficile) infection, using such compositions.

### BACKGROUND

Clostridioides difficile (formerly Clostridium difficile) infection (CDI) is typically treated with antibiotics, which perturbs the gut microbiome, thereby increasing susceptibility to recurrent CDI (rCDI). For patients who have experienced CDI recurrences, fecal microbiota transplant (FMT) and other donor-derived products have been successful in preventing further recurrences of CDI. The composition and quality attributes of these donor-derived procedures are inherently variable, they have occasionally resulted in transmission of harmful pathogens from the human donors of the feces (DeFilipp et al., 2019), the procedure requires extensive and costly donor screening (Craven et al., 2017), and the precise components of the gastrointestinal microbiota that provide resistance against C. difficile remain undetermined (Khanna et al., 2021; McDonald et al., 2018).

### SUMMARY

Provided herein are compositions and methods for the treatment and/or prevention of C. difficile infection. Various factors including antibiotic usage can induce dysbiosis of the gastrointestinal tract, which may allow for colonization by pathogenic microorganisms, such as C. difficile. Such colonization or pathogenic infection can lead to a variety of adverse effects in the subject including diarrhea, which is one of the primary symptoms characteristic of C. difficile infection (CDI). Following administration of an 8-strain bacterial mixture to subjects at risk of recurrent C. difficile infection, it was observed that certain strains of the mixture were associated with non-recurrence, suggesting a role for these bacterial strains specifically in suppression of C. difficile. These and other bacterial strains were also significantly associated with production of short-chain fatty acids (SCFAs) including acetate, butyrate, and valerate, and 2° bile acids including ursodeoxycholic acid, which inhibit C. difficile growth. See, e.g., Gregory et al., PLoS Pathog. 2021. 17(10):e1009959; Weingarden et al., J Clin Gastroenterol. 2016. 50 (8): 624-630. Accordingly, some aspects of the disclosure relate to methods of administering one or more bacterial strains belonging to the species Clostridium bolteae, Anaerotruncus colihominis, and/or Flavonifractor plautii, where one or more of the bacterial strains are detected in a fecal sample obtained from the subject after administration.

Some aspects relate to compositions comprising or consisting of bacterial strains including Clostridium bolteae, Anaerotruncus colihominis, and/or Flavonifractor plautii. Moreover, certain endogenous taxa not present in the administered mixture, such as Bifidobacterium longum and Lachnospiraceae bacterium, were also associated with non-recurrence of C. difficile infection, suggesting a role for these bacteria in suppressing C. difficile, particularly in combination with one or more administered strains. Thus, other aspects relate to compositions comprising or consisting of Bifidobacterium longum and Lachnospiraceae bacterium in combination with one or more other strains, such as Clostridium bolteae, Anaerotruncus colihominis, and/or Flavonifractor plautii.

Accordingly, some aspects relate to a method for treating or preventing recurrent Clostridioides difficile infection (rCDI) in a subject, the method comprising administering to the subject: (a) a bacterial strain belonging to the species Clostridium bolteae; (b) a bacterial strain belonging to the species Anaerotruncus colihominis; and/or (c) a bacterial strain belonging to the species Flavonifractor plautii; wherein at least one of the bacterial strains is detected in a fecal sample obtained from the subject at least two weeks after administration of the bacterial strains.

In some embodiments, at least one of the bacterial strains is detected in a fecal sample obtained from the subject at least 3, 4, 5, 6, 7, 8, 9, 10, 11, 12, 13, 14, 15, 16, 17, 18, 19, 20, 21, 22, 23, or 24 weeks after administration of the bacterial strains.

In some embodiments, the method comprises administering: (a) the bacterial strain belonging to the species Clostridium bolteae; (b) the bacterial strain belonging to the species Anaerotruncus colihominis; (c) the bacterial strain belonging to the species Flavonifractor plautii; (d) a bacterial strain belonging to the species Sellimonas intestinalis; (e) a bacterial strain belonging to the species Clostridium symbiosum; (f) a bacterial strain belonging to the species Blautia producta; (g) a bacterial strain belonging to the species Dorea longicatena; and (h) a bacterial strain belonging to the species Clostridium innocuum.

In some embodiments, the bacterial strain belonging to the species Flavonifractor plautii is detected in a fecal sample obtained from the subject at least two weeks after administration of the bacterial strains.

In some embodiments, the method further comprises measuring the abundance of the bacterial strains belonging to species Clostridium bolteae, Anaerotruncus colihominis, and/or Flavonifractor plautii in a fecal sample obtained from the subject at least two weeks after administration of the bacterial strains. In some embodiments, the method further comprises: (i) identifying a bacterial strain belonging to the species Clostridium bolteae, Anaerotruncus colihominis, or Flavonifractor plautii as not having colonized the subject, if the bacterial strain is not detected in the fecal sample; and (ii) administering to the subject one or more additional doses of the bacterial strain that did not colonize the subject.

Some aspects relate to a method for treating or preventing recurrent Clostridioides difficile infection (rCDI) in a subject, the method comprising administering to the subject: (a) a bacterial strain comprising a 16S rDNA sequence with at least 97% sequence identity to the nucleotide sequence of SEQ ID NO: 1; (b) a bacterial strain comprising a 16S rDNA sequence with at least 97% sequence identity to the nucleotide sequence of SEQ ID NO: 2; and/or (c) a bacterial strain comprising a 16S rDNA sequence with at least 97% sequence identity to the nucleotide sequence of SEQ ID NO: 8; wherein at least one of the bacterial strains is detected in a fecal sample obtained from the subject at least two weeks after administration of the bacterial strains. In some embodiments, (a) the bacterial strain of (a) comprises a 16S rDNA sequence with at least 99% sequence identity to the nucleotide sequence of SEQ ID NO: 1; (b) the bacterial strain of (b) comprises a 16S rDNA sequence with at least 99% sequence identity to the nucleotide sequence of SEQ ID NO: 2; and (c) the bacterial strain of (c) comprises a 16S rDNA sequence with at least 99% sequence identity to the nucleotide sequence of SEQ ID NO: 8.

In some embodiments, at least one of the bacterial strains is detected in a fecal sample obtained from the subject at least 3, 4, 5, 6, 7, 8, 9, 10, 11, 12, 13, 14, 15, 16, 17, 18, 19, 20, 21, 22, 23, or 24 weeks after administration of the bacterial strains.

In some embodiments, the method comprises administering: (a) a bacterial strain comprising a 16S rDNA sequence with at least 97% sequence identity to the nucleotide sequence of SEQ ID NO: 1; (b) a bacterial strain comprising a 16S rDNA sequence with at least 97% sequence identity to the nucleotide sequence of SEQ ID NO: 2; (c) a bacterial strain comprising a 16S rDNA sequence with at least 97% sequence identity to the nucleotide sequence of SEQ ID NO: 8; (d) a bacterial strain comprising a 16S rDNA sequence with at least 97% sequence identity to the nucleotide sequence of SEQ ID NO: 3; (e) a bacterial strain comprising a 16S rDNA sequence with at least 97% sequence identity to the nucleotide sequence of SEQ ID NO: 4; (f) a bacterial strain comprising a 16S rDNA sequence with at least 97% sequence identity to the nucleotide sequence of SEQ ID NO: 5; (g) a bacterial strain comprising a 16S rDNA sequence with at least 97% sequence identity to the nucleotide sequence of SEQ ID NO: 6; and (h) a bacterial strain comprising a 16S rDNA sequence with at least 97% sequence identity to the nucleotide sequence of SEQ ID NO: 7.

In some embodiments: (a) the bacterial strain of (a) comprises a 16S rDNA sequence with at least 99% sequence identity to the nucleotide sequence of SEQ ID NO: 1; (b) the bacterial strain of (b) comprises a 16S rDNA sequence with at least 99% sequence identity to the nucleotide sequence of SEQ ID NO: 2; (c) the bacterial strain of (c) comprises a 16S rDNA sequence with at least 99% sequence identity to the nucleotide sequence of SEQ ID NO: 8; (d) the bacterial strain of (d) comprises a 16S rDNA sequence with at least 99% sequence identity to the nucleotide sequence of SEQ ID NO: 3; (e) the bacterial strain of (e) comprises a 16S rDNA sequence with at least 99% sequence identity to the nucleotide sequence of SEQ ID NO: 4; (f) the bacterial strain of (f) comprises a 16S rDNA sequence with at least 99% sequence identity to the nucleotide sequence of SEQ ID NO: 5; (g) the bacterial strain of (g) comprises a 16S rDNA sequence with at least 99% sequence identity to the nucleotide sequence of SEQ ID NO: 6; and (h) the bacterial strain of (h) comprises a 16S rDNA sequence with at least 99% sequence identity to the nucleotide sequence of SEQ ID NO: 7.

In some embodiments, the bacterial strain of (c) is detected in a fecal sample obtained from the subject at least two weeks after administration of the bacterial strains. In some embodiments, the method further comprises measuring the abundance of the bacterial strain of (a), (b), and/or (c) in a fecal sample obtained from the subject at least two weeks after administration of the bacterial strains. In some embodiments, the bacterial strain of (a), (b), and/or (c) as not having colonized the subject, if the bacterial strain is not detected in the fecal sample; and (ii) administering to the subject one or more additional doses of the bacterial strain that did not colonize the subject.

In some embodiments, the bacterial strains are comprised in a pharmaceutical composition comprising the bacterial strains and a pharmaceutically acceptable excipient. In some embodiments, the pharmaceutical composition is administered orally or rectally. In some embodiments, the pharmaceutical composition is administered orally. In some embodiments, the pharmaceutical composition is formulated for delivery to the intestine. In some embodiments, the pharmaceutical composition is formulated for delivery to the colon. In some embodiments, the pharmaceutical composition comprises one or more enteric polymers.

In some embodiments, the pharmaceutical composition is administered to the subject as multiple doses over multiple days. In some embodiments, the subject is administered about 1.6×10⁹ colony-forming units (CFUs) of the bacterial strains per day. In some embodiments, the subject is administered a total of about 2.2×10¹⁰ colony-forming units (CFUs) of the bacterial strains. In some embodiments, the subject is administered about 8.0×10⁹ colony-forming units (CFUs) of the bacterial strains per day. In some embodiments, the subject is administered about 1.1×10¹¹ colony-forming units (CFUs) of the bacterial strains.

In some embodiments, the method comprises administering an antibiotic to the subject prior to administration of the pharmaceutical composition. In some embodiments, the antibiotic is vancomycin.

In some embodiments, the subject is a human.

### BRIEF DESCRIPTION OF THE DRAWINGS

The accompanying drawings, which constitute a part of this specification, illustrate several embodiments of the disclosure and together with the description, serve to explain the principles of the disclosure.

FIG. 1 shows the recruitment, dosing, monitoring, and endpoints of a double-blind placebo-controlled Phase 2 clinical trial in which an 8-strain bacterial mixture (VE303) was administered to subjects at risk of recurrent C. difficile infection (rCDI). Subjects were randomized into treatment groups: VE303 high dose (1.1×10¹¹ CFU), VE303 low dose (2.2×10¹⁰ CFUs), and placebo control.

FIGS. 2A and 2B show species alpha diversity over time and in recurrent v. non-recurrent subjects. FIG. 2A shows line plots of species alpha diversity represented by the Shannon Index over time for placebo, VE303 low-dose, and VE303 high-dose groups over time. The thick lines represent the median diversity across subjects at each timepoint, and the error bars represent the median absolute deviation. Braces show treatment groups and timepoints being compared, and asterisks show statistically significant differences between compared points as indicated by braces (Table E3-1). FIG. 2B shows that species alpha diversity increased in non-recurrent versus recurrent subjects by the end of dosing (p=0.02; linear mixed effects [LME] model). The Delta (relative diversity change) increased with dose and was highest for the VE303 high-dose group (Placebo: NS, VE303 low-dose: NS, VE303 high-dose: p=0.08; LME). The box-and-whisker plots depict the median, interquartile range, and 95% confidence intervals in the mean diversity per subject over the post-antibiotic and VE303 or placebo dosing period.

FIGS. 3A-3C show associations between bacterial strains and recurrence or non-recurrence of Clostridium difficile infection, or abundance of other pathogens. FIG. 3A shows a volcano plot representing the results of a linear mixed effects (LME) model to relate bacterial species dynamics through Day 14 with CDI recurrence or non-recurrence of disease in the VE303-dosed groups. Species in the right half of the plot are associated with non-recurrence, and those in the left half are associated with recurrence; species above the dashed line have model-adjusted p-values <0.2. Bacterial strains present in VE303 are indicated. FIG. 3B shows associations (p-adjust<0.25, linear mixed effects (LME) model) between bacterial species in subjects dosed with VE303 or placebo and the recurrence or non-recurrence of C. difficile infection. Bars are shaded according to the taxonomic class of each organism. Species associated with non-recurrence include VE303-08 (Flavonifractor sp.), VE303-02 (Anaerotruncus sp.), and VE303-01 (Clostridium bolteae), whereas species associated with recurrence include putative pathogenic organisms from the phylum Proteobacteria. FIG. 3C shows correlations between the relative abundance (RA) of VE303 strains and pathogenic taxa (Klebsiella, Kluyvera, and Veillonella).

FIGS. 4A-4E show effect-size bar plots for all organisms that have significant associations with recurrence or non-recurrence of disease (unadjusted p-value <0.05) at species (FIG. 4A), genus (FIG. 4B), family (FIG. 4C), order (FIG. 4D), and class (FIG. 4E) level. Taxa in the right half of each plot (positive effect size) are associated with non-recurrence; those in the left half of the plot (negative effect size) are associated with recurrence, and bars are colored according to taxonomic class. Vertical annotations on the right indicate significant model correlations with total VE303 abundance up to Day 14. Boxes to the right of each graph indicate positive or negative correlations with VE303. Statistically significant levels of correlations with VE303 are indicated by asterisks.

FIGS. 5A-5F show effects of VE303 administration on the abundance of primary and secondary bile acids (BA). FIG. 5A shows the abundance of primary bile acids (top row) and secondary bile acids (bottom row) over time across treatment groups, as measured by Log-transformed total concentrations (ng/mg) of primary BA and secondary BA in the stool of subjects over time. At each timepoint, right boxes indicate the concentrations for subjects who did not have an on-study CDI recurrence, and left boxes indicate the concentrations for subjects who did have an on-study CDI recurrence. The dose group depicts the concentrations in all VE303-dosed subjects. The box-and-whisker plots depict the median, interquartile range, and 95% confidence intervals of the log-transformed metabolite concentrations at each timepoint. FIGS. 5B-5F shows heatmaps of individual bile acids over time for each subject. The z-score for each row (individual bile acids) is plotted to show the changes in each bile acid over time, at Screening (FIG. 5B), Day 1 (FIG. 5C), Day 7 (FIG. 5D), Day 14 (FIG. 5E), Day 56 (FIG. 5F). Samples are organized by treatment group and by study timepoint. Each subject is annotated by whether they had an on-study CDI recurrence (light gray, R) or did not have an on-study CDI recurrence (dark gray, NR). The primary BAs include CA, CDCA, TCA, TCDCA, GCA, GCDCA. The secondary BAs include DCA, LCA, 3-oxoLCA, TLCA, TDCA, GLCA, GDCA, UDCA, TUDCA, GUDCA, and Isoallo-+IsoLCA.

FIGS. 6A-6F show effects of VE303 administration on the abundance of short-chain fatty acids, including acetic acid, succinic acid, propionic acid, isobutyric acid, butyric acid, 2-methyl-butyric acid, isovaleric acid, valeric acid, and caproic acid (hexanoic acid). FIG. 6A shows the abundance of short-chain fatty acids (SCFAs) over time across treatment groups, as measured by Log-transformed total concentrations (ng/mg) of SCFAs in the stool of subjects over time. At each timepoint, right boxes indicate the concentrations for subjects who did not have an on-study CDI recurrence, and left boxes indicate the concentrations for subjects who did have an on-study CDI recurrence. The dose group depicts the concentrations in all VE303-dosed subjects, separated by those who experienced recurrence (left) and those who did not (right). The box-and-whisker plots depict the median, interquartile range, and 95% confidence intervals of the log-transformed metabolite concentrations at each timepoint. FIGS. 6B-6F shows heatmaps of individual SCFAs over time for each subject. The z-score for each row (individual SCFA) is plotted to show the changes in each SCFA over time, at Screening (FIG. 6B), Day 1 (FIG. 6C), Day 7 (FIG. 6D), Day 14 (FIG. 6E), Day 56 (FIG. 6F). Samples are organized by treatment group and by study timepoint. Each subject is annotated by whether they had an on-study CDI recurrence (R) or did not have an on-study CDI recurrence (NR).

FIG. 7 shows significant relationships (p-adjust<0.05, LME model) between bacterial strains of VE303 and production of the indicated short-chain fatty acids (SCFAs) and secondary bile acids (BA).

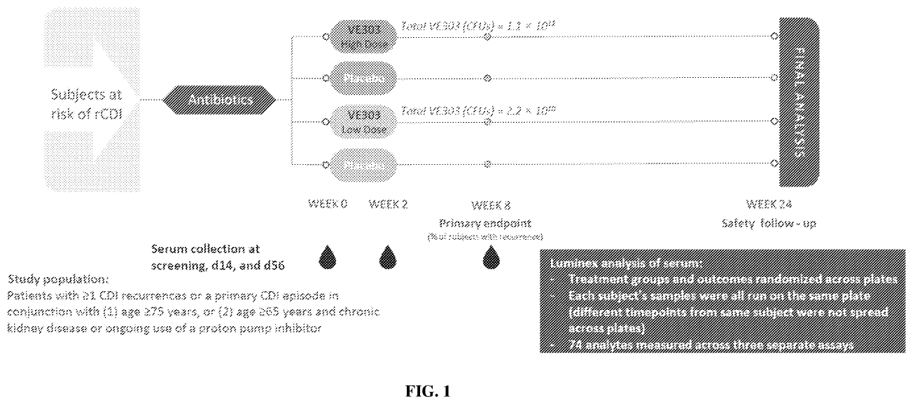

### DETAILED DESCRIPTION

Provided herein are compositions and methods for treatment and/or prevention of Clostridioides difficile (formerly Clostridium difficile) infection (CDI). Administration of an 8-strain bacterial mixture to human subjects at risk of recurrent C. difficile infection revealed significant associations between certain bacterial taxa, some present in the bacterial mixture, and others absent from the mixture, and reduced risk of C. difficile infection recurrence. Moreover, certain strains were found to be associated with increased abundance of short-chain fatty acids (SCFAs) and secondary bile acids (BAs). Without wishing to be limited by any particular theory, SCFAs and secondary BAs are thought to be protective in C. difficile infection, both by directly inhibiting C. difficile replication and indirectly by alleviating inflammation associated with C. difficile infection. Without wishing to be bound by a particular theory, it is expected that colonization by these taxa is indicative of reduced risk of C. difficile infection recurrence, whereas a lack of colonization indicates an elevated risk of recurrence. Administering an 8-strain bacterial mixture to a subject such that one or more of these bacterial strains colonize the subject (as evidenced by their detection in fecal samples from the subject) therefore allows for a reduction in the likelihood of C. difficile infection recurrence. Accordingly, combinations of bacterial strains as described herein are useful for suppressing C. difficile infection.

In some embodiments of methods for treating or preventing rCDI in a subject, one or more bacterial strains belonging to species independently selected from Clostridium bolteae, Anaerotruncus colihominis, and Flavonifractor plautii colonize the gastrointestinal tract of the subject. In some embodiments of methods for treating or preventing rCDI in a subject, one or more of (i) a bacterial strain comprising a 16S rDNA sequence with at least 97% sequence identity to SEQ ID NO: 1, (ii) a bacterial strain comprising a 16S rDNA sequence with at least 97% sequence identity to SEQ ID NO: 2, and (iii) a bacterial strain comprising a 16S rDNA sequence with at least 97% sequence identity to SEQ ID NO: 8 colonize the gastrointestinal tract of the subject. In some embodiments, a bacterial strain belonging to the species Clostridium bolteae colonizes the gastrointestinal tract. In some embodiments, a bacterial strain belonging to the species Anaerotruncus colihominis colonizes the gastrointestinal tract. In some embodiments, a bacterial strain belonging to the species Flavonifractor plautii colonizes the gastrointestinal tract. In some embodiments, a bacterial strain comprising a 16S rDNA sequence with at least 97% sequence identity to SEQ ID NO: 1 colonizes the gastrointestinal tract. In some embodiments, a bacterial strain comprising a 16S rDNA sequence with at least 97% sequence identity to SEQ ID NO: 2 colonizes the gastrointestinal tract. In some embodiments, a bacterial strain comprising a 16S rDNA sequence with at least 97% sequence identity to SEQ ID NO: 8 colonizes the gastrointestinal tract. In some embodiments, at least one of the bacterial strains colonizes an intestine of the subject. In some embodiments, at least one of the bacterial strains colonizes the colon.

In some embodiments, at least one of the bacterial strains is detected in a fecal sample obtained from the subject after administration of the bacterial strains. In some embodiments, a bacterial strain belonging to the species Clostridium bolteae is detected in the fecal sample. In some embodiments, a bacterial strain belonging to the species Anaerotruncus colihominis is detected in the fecal sample. In some embodiments, a bacterial strain belonging to the species Flavonifractor plautii is detected in the fecal sample. In some embodiments, a bacterial strain comprising a 16S rDNA sequence with at least 97% sequence identity to SEQ ID NO: 1 is detected in the fecal sample. In some embodiments, a bacterial strain comprising a 16S rDNA sequence with at least 97% sequence identity to SEQ ID NO: 2 is detected in the fecal sample. In some embodiments, a bacterial strain comprising a 16S rDNA sequence with at least 97% sequence identity to SEQ ID NO: 8 is detected in the fecal sample.

In some embodiments, the method further comprises measuring the abundance of one or more bacterial strains belonging to species independently selected from Clostridium bolteae, Anaerotruncus colihominis, and Flavonifractor plautii in a fecal sample obtained from the subject after administration of the bacterial strains. In some embodiments, the method further comprises measuring the abundance of one or more of (i) a bacterial strain comprising a 16S rDNA sequence with at least 97% sequence identity to SEQ ID NO: 1, (ii) a bacterial strain comprising a 16S rDNA sequence with at least 97% sequence identity to SEQ ID NO: 2, and (iii) a bacterial strain comprising a 16S rDNA sequence with at least 97% sequence identity to SEQ ID NO: 8 in a fecal sample obtained from the subject after administration of the bacterial strains.

In some embodiments, the abundance of a bacterial strain belonging to the species Clostridium bolteae is measured. In some embodiments, the abundance of a bacterial strain belonging to the species Anaerotruncus colihominis is measured. In some embodiments, the abundance of a bacterial strain belonging to the species Flavonifractor plautii is measured. In some embodiments, the abundance of a bacterial strain comprising a 16S rDNA sequence with at least 97% sequence identity to SEQ ID NO: 1 is measured. In some embodiments, the abundance of a bacterial strain comprising a 16S rDNA sequence with at least 97% sequence identity to SEQ ID NO: 2 is measured. In some embodiments, the abundance of a bacterial strain comprising a 16S rDNA sequence with at least 97% sequence identity to SEQ ID NO: 8 is measured.

Any suitable method may be used for detection or quantifying abundance of one or more bacterial strains in a sample. Example 1, for instance, described genomic markers for bacterial strains. Deep sequencing of 16S rRNA (or 16S rDNA) sequences present in a sample may also be used to detect or assay abundance of bacterial strains having particular 16S rRNA or 16S rDNA sequences. As a further example, a sample may be cultured to identify bacterial strains present therein.

In some embodiments, the fecal sample is obtained from the subject at least 1, 2, 3, 4, 5, 6, 7, 8, 9, 10, 11, 12, 13, 14, 15, 16, 17, 18, 19, 21, 22, 23, or 24 weeks after administration of the bacterial strains. In some embodiments, the fecal sample is obtained from the subject at least 1, 2, 3, 4, 5, 6, 7, 8, 9, 10, 11, 12, 13, 14, 15, 16, 17, 18, 19, 21, 22, 23, 24, 25, 26, 27, 28, 29, 30, or 31 days after administration of the bacterial strains.

In some embodiments, the timing of obtaining one or more fecal samples is calculated starting from administration of the first dose of bacterial strains (i.e., 1 week is 7 days after the day on which the first dose is administered). In some embodiments, the timing of obtaining one or more fecal samples is calculated starting from administration of the last dose of a predefined dosing regimen (i.e., 1 week is 7 days after the day on which the last dose is administered).

In some embodiments, the fecal sample is obtained after administration of the last dose of a predefined dosing regimen. In some embodiments, the fecal sample is obtained within 7 or fewer, 6 or fewer, 5 or fewer, 4 or fewer, 3 or fewer, 2 or fewer, or 1 day(s) after administration of the last dose of a predefined dosing regimen.

In some embodiments, the bacterial strain(s) are detected over a period of at least 1, 2, 3, 4, 5, 6, 7, 8, 9, 10, 11, 12, 13, 14, 15, 16, 17, 18, 19, or 20 weeks following administration of the bacterial strains. In some embodiments, the duration over which bacterial strains are detected is calculated starting from administration of the first dose of bacterial strains (i.e., 1 week is 7 days after the day on which the first dose is administered). In some embodiments, the duration over which bacterial strains are detected is calculated starting from administration of the last dose of a specified dosing regimen (i.e., 1 week is 7 days after the day on which the last dose is administered).

In some embodiments, fecal samples are obtained from the subject at regular intervals for at least 1, 2, 3, 4, 5, 6, 7, 8, 9, 10, 11, 12, 13, 14, 15, 16, 17, 18, 19, or 20 weeks, and assayed for presence of bacterial strains. In some embodiments, fecal samples are collected weekly and assayed to determine the presence of bacterial strains.

In some embodiments, fecal samples are obtained from the subject at regular intervals for at least 1, 2, 3, 4, 5, 6, 7, 8, 9, 10, 11, 12, 13, 14, 15, 16, 17, 18, 19, or 20 weeks, and assayed for abundance of bacterial strains. In some embodiments, fecal samples are collected weekly and assayed to determine the abundance of bacterial strains.

In some embodiments, the method further comprises identifying the subject as being at elevated risk for recurrence of C. difficile infection and administering a therapy to the subject. In some embodiments, the subject is identified as being at elevated risk for recurrence of C. difficile infection, where the subject has not been colonized by one or more bacterial strains. In some embodiments, the method comprises identifying the subject as not having been colonized by a bacterial strain belonging to the species Clostridium bolteae. In some embodiments, the method comprises identifying the subject as not having been colonized by a bacterial strain belonging to the species Anaerotruncus colihominis. In some embodiments, the method comprises identifying the subject as not having been colonized by a bacterial strain belonging to the species Flavonifractor plautii. In some embodiments, the method comprises identifying the subject as not having been colonized by a bacterial strain comprising a 16S rDNA sequence with at least 97% sequence identity to SEQ ID NO: 1. In some embodiments, the method comprises identifying the subject as not having been colonized by a bacterial strain comprising a 16S rDNA sequence with at least 97% sequence identity to SEQ ID NO: 2. In some embodiments, the method comprises identifying the subject as not having been colonized by a bacterial strain comprising a 16S rDNA sequence with at least 97% sequence identity to SEQ ID NO: 8.

In some embodiments, the therapy is one or more additional doses of the bacterial strain that was identified as not having colonized the subject. In some embodiments, the therapy is administration of an antibiotic. In some embodiments, the therapy comprises administration of one or more doses of an antibiotic, followed by administration of one or more additional doses of the bacterial strain that was identified as not having colonized the subject. In some embodiments, the antibiotic is selected from vancomycin, kanamycin, gentamicin, colistin, metronidazole, clindamycin, fidaxomicin, penicillin, streptomycin, and cefoperazone. In some embodiments, the antibiotic is vancomycin.

This disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Microbiomes are present, for example in mammalian subjects, on the skin, within the gastrointestinal tract (i.e., the gut), within the oral cavity, and within the vaginal tract of female subjects, and comprise bacteria, archaea, protists, fungi, and viruses. In some instances, the species present in a microbiome benefit the subject by performing useful or necessary functions, such as aiding in the digestion of food in the intestinal tract of the subject, protecting the body from penetration by pathogenic microbes, and promoting immunological development. Organisms within the microbiota that perform these functions may be referred to as symbiotic or commensal organisms because they exist in the subject without harming, and, in some cases, actually benefit the host. In dysbiosis, a state of imbalance of the microbiome of a subject, the normal microbiome of the subject is perturbed or damaged, which may lead to a variety of diseases and/or disorders. In some embodiments, the species that normally dominate the microbiome become underrepresented (e.g., commensal or symbiotic species) and species which are normally underrepresented (e.g., opportunistic species) become overrepresented. See also Petersen et al., “Defining dysbiosis and its influence on host immunity and disease.” Cell Microbiol. (2014) 16(7):1024-1033.

### Bacterial Combinations

Some aspects relate to combinations of purified bacterial strains. Some embodiments relate to compositions comprising a purified bacterial mixture comprising two or more bacterial strains. In some embodiments, the composition is a combination product. “Combination product,” as used herein, has the definition specified in 21 CFR 3.2(e). At the time of filing the instant specification, “combination product” defined under 21 CFR 3.2 (e) includes: (1) A product comprised of two or more regulated components, i.e., drug/device, biologic/device, drug/biologic, or drug/device/biologic, that are physically, chemically, or otherwise combined or mixed and produced as a single entity; (2) Two or more separate products packaged together in a single package or as a unit and comprised of drug and device products, device and biological products, or biological and drug products; (3) A drug, device, or biological product packaged separately that according to its investigational plan or proposed labeling is intended for use only with an approved individually specified drug, device, or biological product where both are required to achieve the intended use, indication, or effect and where upon approval of the proposed product the labeling of the approved product would need to be changed, e.g., to reflect a change in intended use, dosage form, strength, route of administration, or significant change in dose; or (4) Any investigational drug, device, or biological product packaged separately that according to its proposed labeling is for use only with another individually specified investigational drug, device, or biological product where both are required to achieve the intended use, indication, or effect.

Some embodiments relate to methods of administering a composition comprising a combination of purified bacterial strains. Some embodiments relate to methods of administering a combination of purified bacterial strains to a subject. Some embodiments relate to kits comprising a combination of purified bacterial strains. The skilled artisan will appreciate that because bacterial strains may colonize the gastrointestinal tract of a subject and exert a therapeutic effect through their presence in the gastrointestinal tract, it is not necessary that all bacterial strains administered to the subject be present in a single dosage form (e.g., a single capsule comprising all bacterial strains of the combination).

In some embodiments, a combination of bacterial strains lacks one or more bacterial strains of a species or having a specified 16S rDNA sequence. In some embodiments, a composition does not comprise one or more bacterial strains of a species or having a specified 16S rDNA sequence. In some embodiments, a kit does not comprise one or more bacterial strains of a species or having a specified 16S rDNA sequence. In some embodiments, a method does not comprise administering a bacterial strain of a species or having a specified 16S rDNA sequence. Compositions may be said not to comprise (i.e., to lack) bacterial strains of a species or having a specified 16S rDNA sequence if bacteria of that species or having the specified 16S rDNA sequence are not detectable in the composition above a limit of detection. Kits may be said not to comprise (i.e., to lack) bacterial strains of a species or having a specified 16S rDNA sequence if bacteria of that species or having the specified 16S rDNA sequence, respectively, are not detectable in any dosage form of the kit. A method may be said not to comprise administering (i.e., to lack administering) bacterial strains of a species or having a specified 16S rDNA sequence if no dosage form administered to a subject comprises a detectable amount of bacterial strains of the species or having the specified 16S rDNA sequence. In each case, it is the combination of bacterial strains contemplated in a composition, kit, or method, that the skilled artisan will evaluate in determining whether a bacterial strain is present or used. In some embodiments of a method that does not comprise administering a specified bacterial strain, the specified bacterial strain is not administered to the subject within 1 day, within 1 week, or within 1 month of administering any other bacterial strain of the method.

Bacterial strains of a combination may be included in separate individual dosage forms, in arbitrary combinations in two or more dosage forms, or all bacterial strains may be included in a single dosage form. When administered as separate dosage forms, the administration form and route may be the same or different for each dosage form. Dosage forms may be administered simultaneously, sequentially, and/or separately. In some embodiments, simultaneous administration of a plurality of dosage forms occurs when all dosage forms of the plurality are administered within a period of 1, 2, 3, 4, or 5 minute(s). In some embodiments, sequential administration of a plurality of dosage forms occurs when all dosage forms of the plurality are administered in series. In some embodiments, separate administration of a plurality of dosage forms occurs when no two dosage forms of the plurality are administered within a period of 1, 2, 3, 4, or 5 minute(s).

It should be appreciated that the terms “bacteria” and “bacterial strains” as used herein are interchangeable. Compositions containing bacterial strains may also be referred to as “live bacterial products” or “live biotherapeutic products.”

Multiple bacterial strains of a combination may belong to the same species. Bacterial strains belonging to the same species may differ in one or more characteristics. Bacterial strains belonging to the same species may be distinguished using one or more of multiple features known in the art, such as colony morphology (e.g., smooth v. rough, flat v. raised, color). Bacterial strains of the same species may also be distinguished on the basis of presence or absence of one or more genes, such as antibiotic resistance genes. Bacterial strains within a species may also be distinguished by variation between 16S rDNA sequences (e.g., having at least 97% and less than 100% identity between 16S rDNA sequences), sequences of additional genes, and/or sequence of the whole genome (or portion thereof).

Methods of detecting bacterial strains of a given species are known in the art, including amplification, sequencing, and identification of 16S rDNA and/or chromosomal DNA isolated from a biological sample (e.g., composition or fecal sample).

In some embodiments, a combination comprises at least 1, at least 2, at least 3, at least 4, at least 5, at least 6, at least 7, or at least 8 bacterial strains. In some embodiments, a combination comprises 2, 3, 4, 5, 6, 7, or 8 bacterial strains. In some embodiments, a combination comprises 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, 1-2, 7-11, 8-11, 9-11, 10-11, 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 0-6, 1-6, 2-6, 3-6, 4-6, 5-6, 1-5, 2-5, 3-5, 4-5, 1-4, 2-4, 3-4, 1-3, 2-3, or 1-2 bacterial strains.

Species and 16S rRNA Sequences of Bacterial Strains

Bacterial strains may be classified phylogenetically with other closely related bacterial strains based on their 16S rRNA (or 16S rDNA) nucleic acid sequences. Methods for determining the identity of specific bacterial species based on their 16S rRNA (or 16S rDNA) nucleic acid sequence are known in the art. See, e.g., Jumpstart Consortium Human Microbiome Project Data Generation Working, G. PLoS One. (2012) 7:e39315.

Bacterial strains may be classified taxonomically as belonging to a particular species. The species to which a bacterial strain belongs may be determined, for instance, by comparing the genome of the strain to a database of bacterial genomes. Additionally or alternatively, the 16S rRNA (or 16S rDNA) sequence may be compared to a database of 16S rRNA (or 16S rDNA) sequences to identify closely related species.

In some embodiments a bacterial combination comprises one or more bacterial strains provided in Table 1. In some embodiments, the combination comprises one or more bacterial strains belonging to the following species: (i) Clostridium bolteae (Lachnoclostridium bolteae, Enterocloster bolteae), (ii) Anaerotruncus colihominis, (iii) Sellimonas intestinalis (Drancourtella massilienses, Ruminococcus torques, Eubacterium fissicatena), (iv) Clostridium symbiosum (Lachnoclostridium symbiosum), (v) Blautia producta (Blautia sp001304935), (vi) Dorea longicatena, (vii) Clostridium innocuum (Erysipelotrichaceae innocuum, Eubacterium innocuum, Absiella innocuum, Longicatena innocuum, Erysipelotrichaceae bacterium), and (viii) Flavonifractor plautii (Clostridium orbiscindens, Subdolinogranulum spp). In some embodiments, the combination comprises at least 2, at least 3, at least 4, at least 5, at least 6, at least 7, or at least 8 bacterial strains (e.g., purified bacterial strains) listed in Table 1. As will be appreciated by one of ordinary skill in the art, a bacterial strain may be closely related to one or more bacterial species. Alternatively or in addition, a bacterial strain may be referred to by one or more bacterial species names, based on changing nomenclature and phylogenetic classification.

In some embodiments, the combination includes Clostridium bolteae. In some embodiments, the bacterial strain referred to as Clostridium bolteae and having a 16S rDNA sequence comprising the nucleic acid sequence of SEQ ID NO: 1 may also be referred to, for example, as Lachnoclostridium bolteae or Enterocloster bolteae. In some embodiments, the combination includes Anaerotruncus colihominis. In some embodiments, the bacterial strain referred to as Anaerotruncus colihominis has a 16S rDNA sequence comprising the nucleic acid sequence of SEQ ID NO: 2. In some embodiments, the combination includes Sellimonas intestinalis. In some embodiments, the bacterial strain referred to as Sellimonas intestinalis and having a 16S rDNA sequence comprising the nucleic acid sequence of SEQ ID NO: 3 may also be referred to, for example, as Drancourtella massilienses, Eubacterium fissicatena, or Ruminococcus torques. In some embodiments, the combination includes Clostridium symbiosum. In some embodiments, the bacterial strain referred to as Clostridium symbiosum and having a 16S rDNA sequence comprising the nucleic acid sequence of SEQ ID NO: 4 may also be referred to, for example, as Lachnoclostridium symbiosum. In some embodiments, the combination includes Blautia producta. In some embodiments, the bacterial strain referred to as Blautia producta and having a 16S rDNA sequence comprising the nucleic acid sequence of SEQ ID NO: 5 may also be referred to, for example, as Blautia sp001304935. In some embodiments, the combination includes Dorea longicatena. In some embodiments, the bacterial strain referred to as Dorea longicatena has a 16S rDNA sequence comprising the nucleic acid sequence of SEQ ID NO: 6. In some embodiments, the combination includes Clostridium innocuum. In some embodiments, the bacterial strain referred to as Clostridium innocuum and having a 16S rDNA sequence comprising the nucleic acid sequence of SEQ ID NO: 7 may also be referred to, for example, as Erysipelotrichaceae bacterium, Erysipelotrichaceae innocuum, Eubacterium innocuum, Absiella innocuum, and Longicatena innocuum. In some embodiments, the combination includes Flavonifractor plautii. In some embodiments, the bacterial strain referred to as Flavonifractor plautii and having a 16S rDNA sequence comprising the nucleic acid sequence of SEQ ID NO: 8 may also be referred to, for example, as Subdoligranulum spp. or Clostridium orbiscindens.

In some embodiments, the combination comprises one or more bacterial strains selected from the group consisting of Clostridium bolteae, Anaerotruncus colihominis, Sellimonas intestinalis, Clostridium symbiosum, Blautia producta, Dorea longicatena, Clostridium innocuum, and Flavonifractor plautii. In some embodiments, the combination comprises Clostridium bolteae, Anaerotruncus colihominis, Sellimonas intestinalis, Clostridium symbiosum, Blautia producta, Dorea longicatena, Clostridium innocuum, and Flavonifractor plautii. In some embodiments, the combination does not comprise bacterial strains other than those belonging to Clostridium bolteae, Anaerotruncus colihominis, Sellimonas intestinalis, Clostridium symbiosum, Blautia producta, Dorea longicatena, Clostridium innocuum, and Flavonifractor plautii.

Some embodiments relate to bacterial strains comprising a 16S rRNA (or 16S rDNA) sequence having a specified percentage identity to a comparator nucleotide sequence (e.g., SEQ ID NOs: 1-8).

The term “identity” refers to a relationship between the sequences of two sequences, as determined by comparing the sequences. Identity also refers to the degree of sequence relatedness between or among sequences as determined by the number of matches between strings of two or more nucleotide sequences. “Percent (%) identity” or “percent (%) sequence identity” as it applies to nucleotide sequences is defined as the percentage of nucleotides in the candidate nucleotide sequence that are identical to the nucleotide sequence of a comparator sequence after aligning the sequences and introducing gaps, as necessary, to achieve the maximum percent identity allowed by the alignment parameters.

The percent sequence identity that a candidate sequence (e.g., as present in a claimed bacterial strain) has to a comparator sequence (e.g., having a SEQ ID NO: specified herein) is calculated by (i) aligning the candidate sequence to the comparator sequence, (ii) determining the number of matching nucleotides between the aligned candidate and comparator sequences, and (iii) dividing the number of matching residues by the length of the comparator sequence, including any gaps introduced into the comparator sequence when the two sequences are aligned.

Where an alignment between two 16S rRNA or 16S rDNA nucleotide sequences is contemplated, the first sequence (e.g., candidate sequence) is aligned to the second sequence (e.g., comparator sequence) using the Needleman-Wunsch algorithm for global alignment of the two sequences. Needleman & Wunsch, J Mol Biol. 1970. 48:443-453. Where two 16S rRNA or 16S rDNA nucleotide sequences are aligned, the alignment uses an DNAFULL substitution scoring matrix, a Gap Open penalty of 10, a Gap Extend penalty of 0.5, and no End Gap penalties. The skilled artisan will appreciate that at the time of filing the instant specification, these parameters are the default parameters of the EMBOSS Needle pairwise comparison tool provided by European Bioinformatics Institute (see ebi.ac.uk). Other suitable alignment programs may be used to obtain a global alignment using these parameters, such as BLAST, or the Needleman-Wunsch algorithm may be implemented in a scripting language (e.g., Python).

The skilled artisan will appreciate that to determine whether a candidate nucleotide sequence comprises a nucleotide sequence with a given percentage sequence identity to a comparator sequence, the denominator (length of comparator sequence plus internal gaps) in calculating sequence identity need not include gaps shown at the ends of the comparator sequence in an alignment, as such gaps are added where a candidate sequence contains additional nucleotides that extend beyond the portions of the candidate sequence that align to the 5′ end or 3′ end of the comparator sequence.

In some embodiments, a combination comprises one or more of (i) a bacterial strain comprising a 16S rDNA sequence with at least 97% sequence identity to SEQ ID NO: 1, (ii) a bacterial strain comprising a 16S rDNA sequence with at least 97% sequence identity to SEQ ID NO: 2, (iii) a bacterial strain comprising a 16S rDNA sequence with at least 97% sequence identity to SEQ ID NO: 3, (iv) a bacterial strain comprising a 16S rDNA sequence with at least 97% sequence identity to SEQ ID NO: 4, (v) a bacterial strain comprising a 16S rDNA sequence with at least 97% sequence identity to SEQ ID NO: 5, (vi) a bacterial strain comprising a 16S rDNA sequence with at least 97% sequence identity to SEQ ID NO: 6, (vii) a bacterial strain comprising a 16S rDNA sequence with at least 97% sequence identity to SEQ ID NO: 7, and (viii) a bacterial strain comprising a 16S rDNA sequence with at least 97% sequence identity to SEQ ID NO: 8.

In some embodiments, a combination comprises (i) a bacterial strain comprising a 16S rDNA sequence with at least 97% sequence identity to SEQ ID NO: 1, (ii) a bacterial strain comprising a 16S rDNA sequence with at least 97% sequence identity to SEQ ID NO: 2, (iii) a bacterial strain comprising a 16S rDNA sequence with at least 97% sequence identity to SEQ ID NO: 3, (iv) a bacterial strain comprising a 16S rDNA sequence with at least 97% sequence identity to SEQ ID NO: 4, (v) a bacterial strain comprising a 16S rDNA sequence with at least 97% sequence identity to SEQ ID NO: 5, (vi) a bacterial strain comprising a 16S rDNA sequence with at least 97% sequence identity to SEQ ID NO: 6, (vii) a bacterial strain comprising a 16S rDNA sequence with at least 97% sequence identity to SEQ ID NO: 7, and (viii) a bacterial strain comprising a 16S rDNA sequence with at least 97% sequence identity to SEQ ID NO: 8.

In some embodiments, a combination does not comprise bacterial strains other than (i) a bacterial strain comprising a 16S rDNA sequence with at least 97% sequence identity to SEQ ID NO: 1, (ii) a bacterial strain comprising a 16S rDNA sequence with at least 97% sequence identity to SEQ ID NO: 2, (iii) a bacterial strain comprising a 16S rDNA sequence with at least 97% sequence identity to SEQ ID NO: 3, (iv) a bacterial strain comprising a 16S rDNA sequence with at least 97% sequence identity to SEQ ID NO: 4, (v) a bacterial strain comprising a 16S rDNA sequence with at least 97% sequence identity to SEQ ID NO: 5, (vi) a bacterial strain comprising a 16S rDNA sequence with at least 97% sequence identity to SEQ ID NO: 6, (vii) a bacterial strain comprising a 16S rDNA sequence with at least 97% sequence identity to SEQ ID NO: 7, and (viii) a bacterial strain comprising a 16S rDNA sequence with at least 97% sequence identity to SEQ ID NO: 8.

In some embodiments, a combination comprises a bacterial strain comprising a 16S rDNA sequence with at least 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.1%, 99.2%, 99.3%, 99.4%, 99.5%, 99.6%, 99.7%, 99.8%, 99.9%, or 100% sequence identity to SEQ ID NO: 1. In some embodiments, the bacterial strain comprises a 16S rDNA sequence with at least 99% sequence identity to SEQ ID NO: 1.

In some embodiments, a combination comprises a bacterial strain comprising a 16S rDNA sequence with at least 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.1%, 99.2%, 99.3%, 99.4%, 99.5%, 99.6%, 99.7%, 99.8%, 99.9%, or 100% sequence identity to SEQ ID NO: 2. In some embodiments, the bacterial strain comprises a 16S rDNA sequence with at least 99% sequence identity to SEQ ID NO: 2.

In some embodiments, a combination comprises a bacterial strain comprising a 16S rDNA sequence with at least 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.1%, 99.2%, 99.3%, 99.4%, 99.5%, 99.6%, 99.7%, 99.8%, 99.9%, or 100% sequence identity to SEQ ID NO: 3. In some embodiments, the bacterial strain comprises a 16S rDNA sequence with at least 99% sequence identity to SEQ ID NO: 3.

In some embodiments, a combination comprises a bacterial strain comprising a 16S rDNA sequence with at least 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.1%, 99.2%, 99.3%, 99.4%, 99.5%, 99.6%, 99.7%, 99.8%, 99.9%, or 100% sequence identity to SEQ ID NO: 4. In some embodiments, the bacterial strain comprises a 16S rDNA sequence with at least 99% sequence identity to SEQ ID NO: 4.

In some embodiments, a combination comprises a bacterial strain comprising a 16S rDNA sequence with at least 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.1%, 99.2%, 99.3%, 99.4%, 99.5%, 99.6%, 99.7%, 99.8%, 99.9%, or 100% sequence identity to SEQ ID NO: 5. In some embodiments, the bacterial strain comprises a 16S rDNA sequence with at least 99% sequence identity to SEQ ID NO: 5.

In some embodiments, a combination comprises a bacterial strain comprising a 16S rDNA sequence with at least 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.1%, 99.2%, 99.3%, 99.4%, 99.5%, 99.6%, 99.7%, 99.8%, 99.9%, or 100% sequence identity to SEQ ID NO: 6. In some embodiments, the bacterial strain comprises a 16S rDNA sequence with at least 99% sequence identity to SEQ ID NO: 6.

In some embodiments, a combination comprises a bacterial strain comprising a 16S rDNA sequence with at least 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.1%, 99.2%, 99.3%, 99.4%, 99.5%, 99.6%, 99.7%, 99.8%, 99.9%, or 100% sequence identity to SEQ ID NO: 7. In some embodiments, the bacterial strain comprises a 16S rDNA sequence with at least 99% sequence identity to SEQ ID NO: 7.

In some embodiments, a combination comprises a bacterial strain comprising a 16S rDNA sequence with at least 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.1%, 99.2%, 99.3%, 99.4%, 99.5%, 99.6%, 99.7%, 99.8%, 99.9%, or 100% sequence identity to SEQ ID NO: 8. In some embodiments, the bacterial strain comprises a 16S rDNA sequence with at least 99% sequence identity to SEQ ID NO: 8.

In some embodiments, a combination comprises a bacterial strain comprising a 16S rDNA sequence with at least 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.1%, 99.2%, 99.3%, 99.4%, 99.5%, 99.6%, 99.7%, 99.8%, 99.9%, or 100% sequence identity to SEQ ID NO: 9. In some embodiments, the bacterial strain comprises a 16S rDNA sequence with at least 99% sequence identity to SEQ ID NO: 9.

In some embodiments, a combination comprises a bacterial strain comprising a 16S rDNA sequence with at least 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.1%, 99.2%, 99.3%, 99.4%, 99.5%, 99.6%, 99.7%, 99.8%, 99.9%, or 100% sequence identity to SEQ ID NO: 10. In some embodiments, the bacterial strain comprises a 16S rDNA sequence with at least 99% sequence identity to SEQ ID NO: 10.

Identifying bacteria by their 16S rRNA (or 16S rDNA) sequences furthermore allows for the identification of additional bacterial strains that are identical or highly similar to the exemplified bacteria. For instance, the 16S rRNA sequences of bacterial strains were used to identify the closest relative (based on percent identity) through whole genome sequencing and Sanger sequencing of the 16S rRNA-encoding (16S rDNA) locus and by comparing these sequences with 16S rRNA databases (Table 1). In addition, based on whole genome sequencing (WGS) and comparing of the whole genome to whole genome (WG) databases, the bacterial strains having 16S rRNA sequences provided by SEQ ID NOs: 1-8 are most closely related to the following bacterial species: Clostridium bolteae, Anaerotruncus colihominis, Sellimonas intestinalis, Clostridium symbiosum, Blautia producta, Dorea longicatena, Clostridium innocuum, Flavonifractor plautii (see, e.g., Table 1). Thus, in one aspect it should be appreciated that each row of Table 1, the bacterial strains are highly similar and/or are identical. In some embodiments, in context of the instant disclosure, the names of bacterial strains within a row of Table 1 can be used interchangeably.

Homologies based on 16S rDNA sequence analysis are presented in Table 1. Table 1 provides the closest known species by sequence identity when the 16S rDNA sequences, or portions thereof, comprising SEQ ID NOs: 1-10 are compared to 16S rDNA sequences of bacterial species available in public databases. Throughout the instant application the bacterial strains associated with 16S rDNA sequences comprising SEQ ID NOs: 1-10 may alternatively or in addition be referred to by any of the additional nucleic acid sequences provided by SEQ ID NOs: 1-10, as shown in Table 1. It should be appreciated that multiple bacterial strains of the compositions described herein may have the same closest related bacterial species. It should be appreciated that the bacterial strains described herein having a 16S rDNA sequence selected from SEQ ID NOs: 1-10 may also be homologous to other strains based on their whole genome sequence, or subset of their whole genome sequence.

Bacterial strains of species listed in Table 1 are available to those of ordinary skill in the art, from repositories such as the American Type Culture Collection (ATCC) and Deutsche Sammlung von Mikroorganismen und Zellkulturen (DSMZ). ATCC is a repository for reference cultures and biological materials, including microorganisms, distributing cultures to scientists worldwide. Similarly, DSMZ is an active collection of biological resources including microorganisms, from which catalogued strains are available. For example, Table 2 provides deposit numbers and GenBank Accession Nos. of 16S rDNA sequences of representative bacterial strains of each species listed in Table 1.

Some aspects of the disclosure relate to combinations comprising at least two bacterial strains (e.g., 2 or 3) belonging to species independently selected from Clostridium bolteae, Anaerotruncus colihominis, and Flavonifractor plautii, and wherein the combination lacks bacterial strains selected from two or more species selected from Sellimonas intestinalis, Clostridium symbiosum, Blautia producta, Dorea longicatena, and Clostridium innocuum. For example, in some embodiments, the combination comprises bacterial strains belonging to Clostridium bolteae and Anaerotruncus colihominis. In some embodiments, the combination comprises bacterial strains belonging to Clostridium bolteae and Flavonifractor plautii. In some embodiments, the combination comprises bacterial strains belonging to Anaerotruncus colihominis and Flavonifractor plautii. In some embodiments, the combination comprises bacterial strains belonging to Clostridium bolteae, Anaerotruncus colihominis, and Flavonifractor plautii. In some embodiments, the combination does not include bacterial strains other than those belonging to Clostridium bolteae and Anaerotruncus colihominis. In some embodiments, the combination does not include bacterial strains other than those belonging to Clostridium bolteae and Flavonifractor plautii. In some embodiments, the combination does not include bacterial strains other than those belonging to Anaerotruncus colihominis and Flavonifractor plautii. In some embodiments, the combination does not include bacterial strains other than those belonging to Clostridium bolteae, Anaerotruncus colihominis, and Flavonifractor plautii.

A combination comprising a bacterial strain of a species may include any number of bacterial strains belonging to that species. For example, in some embodiments, a combination comprising Flavonifractor plautii may comprise 1, 2, 3, 4, 5, 6, 7, 8, 9, 10, 11, 12, 13, 14, 15, 16, 17, 18, 19, or 20 different bacterial strains belonging to the species Flavonifractor plautii. In some embodiments, a combination comprising Clostridium bolteae may comprise 1, 2, 3, 4, 5, 6, 7, 8, 9, 10, 11, 12, 13, 14, 15, 16, 17, 18, 19, or 20 bacterial strains belonging to Clostridium bolteae. In some embodiments, a combination comprising Anaerotruncus colihominis may comprise 1, 2, 3, 4, 5, 6, 7, 8, 9, 10, 11, 12, 13, 14, 15, 16, 17, 18, 19, or 20 bacterial strains belonging to Anaerotruncus colihominis.

In some embodiments, at least two species selected from Sellimonas intestinalis, Clostridium symbiosum, Blautia producta, Dorea longicatena, and Clostridium innocuum are not present in the combination. In some embodiments, at least three species selected from Sellimonas intestinalis, Clostridium symbiosum, Blautia producta, Dorea longicatena, and Clostridium innocuum are not present in the combination. In some embodiments, at least four species selected from Sellimonas intestinalis, Clostridium symbiosum, Blautia producta, Dorea longicatena, and Clostridium innocuum are not present in the combination. In some embodiments, no bacterial strains belonging to Sellimonas intestinalis, no bacterial strains belonging to Clostridium symbiosum, no bacterial strains belonging to Blautia producta, no bacterial strains belonging to Dorea longicatena, and no bacterial strains belonging to Clostridium innocuum, are present in the combination.

Combinations of bacteria may include one or more additional bacterial strains. Additional bacterial strains may be taxonomically or phylogenetically related to bacterial strains of Bifidobacterium longum and/or Lachnospiraceae bacterium. For example, in some embodiments, one or more of the additional bacterial strains belongs to the same phylum, class, order, family, genus, or species as another strain described herein. In some embodiments, one or more additional bacterial strains belong to the genus Lachnospiraceae. In some embodiments, one or more additional bacterial strains belong to the species Lachnospiraceae bacterium. In some embodiments, one or more additional bacterial strains belong to the genus Bifidobacterium. In some embodiments, one or more additional bacterial strains belong to the species Bifidobacterium longum.

In some embodiments, the combination comprises 2, 3, 4, 5, 6, 7, 8, 9, 10, 11, 12, 13, 14, 15, 16, 17, 18, 19, or 20 additional bacterial strains belonging to the genus Lachnospiraceae. In some embodiments, the combination comprises 2, 3, 4, 5, 6, 7, 8, 9, 10, 11, 12, 13, 14, 15, 16, 17, 18, 19, or 20 additional bacterial strains belonging to the species Lachnospiraceae bacterium. In some embodiments, the combination comprises 2, 3, 4, 5, 6, 7, 8, 9, 10, 11, 12, 13, 14, 15, 16, 17, 18, 19, or 20 additional bacterial strains belonging to the genus Bifidobacterium. In some embodiments, the combination comprises 2, 3, 4, 5, 6, 7, 8, 9, 10, 11, 12, 13, 14, 15, 16, 17, 18, 19, or 20 additional bacterial strains belonging to the species Bifidobacterium longum.

Some aspects relate to combinations comprising bacterial strains belonging to each of Clostridium bolteae, Anaerotruncus colihominis, Sellimonas intestinalis, Clostridium symbiosum, Blautia producta, Dorea longicatena, Clostridium innocuum, and Flavonifractor plautii, and one or more additional bacterial strains selected from Lachnospiraceae bacterium and Bifidobacterium longum. In some embodiments, the combination comprises 2, 3, 4, 5, 6, 7, 8, 9, 10, 11, 12, 13, 14, 15, 16, 17, 18, 19, or 20 additional bacterial strains belonging to the species Lachnospiraceae bacterium. In some embodiments, the combination comprises 2, 3, 4, 5, 6, 7, 8, 9, 10, 11, 12, 13, 14, 15, 16, 17, 18, 19, or 20 additional bacterial strains belonging to the species Bifidobacterium longum.

In some embodiments, the combination comprises bacterial strains belonging to Clostridium bolteae, Anaerotruncus colihominis, Sellimonas intestinalis, Clostridium symbiosum, Blautia producta, Dorea longicatena, Clostridium innocuum, Flavonifractor plautii, and Bifidobacterium longum. In some embodiments, the combination comprises bacterial strains belonging to Clostridium bolteae, Anaerotruncus colihominis, Sellimonas intestinalis, Clostridium symbiosum, Blautia producta, Dorea longicatena, Clostridium innocuum, Flavonifractor plautii, and Lachnospiraceae bacterium. In some embodiments, the combination comprises bacterial strains belonging to Clostridium bolteae, Anaerotruncus colihominis, Sellimonas intestinalis, Clostridium symbiosum, Blautia producta, Dorea longicatena, Clostridium innocuum, Flavonifractor plautii, Bifidobacterium longum, and Lachnospiraceae bacterium.

In some embodiments, the combination does not include bacterial strains other than those belonging to Clostridium bolteae, Anaerotruncus colihominis, Sellimonas intestinalis, Clostridium symbiosum, Blautia producta, Dorea longicatena, Clostridium innocuum, Flavonifractor plautii, and Bifidobacterium longum. In some embodiments, the combination does not include bacterial strains other than those belonging to Clostridium bolteae, Anaerotruncus colihominis, Sellimonas intestinalis, Clostridium symbiosum, Blautia producta, Dorea longicatena, Clostridium innocuum, Flavonifractor plautii, and Lachnospiraceae bacterium. In some embodiments, the combination does not include bacterial strains other than those belonging to Clostridium bolteae, Anaerotruncus colihominis, Sellimonas intestinalis, Clostridium symbiosum, Blautia producta, Dorea longicatena, Clostridium innocuum, Flavonifractor plautii, Bifidobacterium longum, and Lachnospiraceae bacterium.

A combination that includes a given species (i.e., comprises a given species, or consists of a combination of species including that given species) may comprise any number of bacterial strains of that species. In some embodiments, a combination that comprises Clostridium bolteae may comprise 2, 3, 4, 5, 6, 7, 8, 9, 10, 11, 12, 13, 14, 15, 16, 17, 18, 19, or 20 bacterial strains of the species Clostridium bolteae. In some embodiments, a combination that comprises Anaerotruncus colihominis may comprise 2, 3, 4, 5, 6, 7, 8, 9, 10, 11, 12, 13, 14, 15, 16, 17, 18, 19, or 20 bacterial strains of the species Anaerotruncus colihominis. In some embodiments, a combination that comprises Sellimonas intestinalis may comprise 2, 3, 4, 5, 6, 7, 8, 9, 10, 11, 12, 13, 14, 15, 16, 17, 18, 19, or 20 bacterial strains of the species Sellimonas intestinalis. In some embodiments, a combination that comprises Clostridium symbiosum may comprise 2, 3, 4, 5, 6, 7, 8, 9, 10, 11, 12, 13, 14, 15, 16, 17, 18, 19, or 20 bacterial strains of the species Clostridium symbiosum. In some embodiments, a combination that comprises Blautia producta may comprise 2, 3, 4, 5, 6, 7, 8, 9, 10, 11, 12, 13, 14, 15, 16, 17, 18, 19, or 20 bacterial strains of the species Blautia producta. In some embodiments, a combination that comprises Dorea longicatena may comprise 2, 3, 4, 5, 6, 7, 8, 9, 10, 11, 12, 13, 14, 15, 16, 17, 18, 19, or 20 bacterial strains of the species Dorea longicatena. In some embodiments, a combination that comprises Clostridium innocuum comprises 2, 3, 4, 5, 6, 7, 8, 9, 10, 11, 12, 13, 14, 15, 16, 17, 18, 19, or 20 bacterial strains of the species Clostridium innocuum. In some embodiments, a combination that comprises Flavonifractor plautii may comprise 2, 3, 4, 5, 6, 7, 8, 9, 10, 11, 12, 13, 14, 15, 16, 17, 18, 19, or 20 bacterial strains of the species Flavonifractor plautii. In some embodiments, a combination that comprises Lachnospiraceae bacterium may comprise 2, 3, 4, 5, 6, 7, 8, 9, 10, 11, 12, 13, 14, 15, 16, 17, 18, 19, or 20 bacterial strains of the species Lachnospiraceae bacterium. In some embodiments, a combination that comprises Bifidobacterium longum may comprise 2, 3, 4, 5, 6, 7, 8, 9, 10, 11, 12, 13, 14, 15, 16, 17, 18, 19, or 20 bacterial strains of the species Bifidobacterium longum.

In some embodiments, the combinations disclosed herein comprise two or more bacterial strains. In some embodiments, the combinations described herein comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or up to 20 total bacterial strains (e.g., purified bacterial strains). In some embodiments, the combinations described herein comprise 3-20, 3-19, 3-18, 3-17, 3-16, 3-15, 3-14, 3-13, 3-12, 3-11, 3-10, 3-9, 3-8, 3-7, 3-6, 3-5, 3-4, 4-20, 4-19, 4-18, 4-17, 4-16, 4-15, 4-14, 4-13, 4-12, 4-11, 4-10, 4-9, 4-8, 4-7, 4-6, 4-5, 5-20, 5-19, 5-18, 5-17, 5-16, 5-15, 5-14, 5-13, 5-12, 5-11, 5-10, 5-9, 5-8, 5-7, 5-6, 6-20, 6-19, 6-18, 6-17, 6-16, 6-15, 6-14, 6-13, 6-12, 6-11, 6-10, 6-9, 6-8, 6-7, 7-20, 7-19, 7-18, 7-17, 7-16, 7-15, 7-14, 7-13, 7-12, 7-11, 7-10, 7-9, 7-8, 8-20, 8-19, 8-18, 8-17, 8-16, 8-15, 8-14, 8-13, 8-12, 8-11, 8-10, 8-9, 9-20, 9-19, 9-18, 9-17, 9-16, 9-15, 9-14, 9-13, 9-12, 9-11, 9-10, 10-20, 10-19, 10-18, 10-17, 10-16, 10-15, 10-14, 10-13, 10-12, 10-11, 11-20, 11-19, 11-18, 11-17, 11-16, 11-15, 11-14, 11-13, 11-12, 12-20, 12-19, 12-18, 12-17, 12-16, 12-15, 12-14, 12-13, 13-20, 13-19, 13-18, 13-17, 13-16, 13-15, 13-14, 14-20, 14-19, 14-18, 14-17, 14-16, 14-15, 15-20, 15-19, 15-18, 15-17, 15-16, 16-20, 16-19, 16-18, 16-17, 17-20, 17-19, 17-18, 18-20, 18-19, or 19-20 bacterial strains. In some embodiments, the combinations comprise a mixture of bacterial strains that consists of 3-20, 3-19, 3-18, 3-17, 3-16, 3-15, 3-14, 3-13, 3-12, 3-11, 3-10, 3-9, 3-8, 3-7, 3-6, 3-5, 3-4, 4-20, 4-19, 4-18, 4-17, 4-16, 4-15, 4-14, 4-13, 4-12, 4-11, 4-10, 4-9, 4-8, 4-7, 4-6, 4-5, 5-20, 5-19, 5-18, 5-17, 5-16, 5-15, 5-14, 5-13, 5-12, 5-11, 5-10, 5-9, 5-8, 5-7, 5-6, 6-20, 6-19, 6-18, 6-17, 6-16, 6-15, 6-14, 6-13, 6-12, 6-11, 6-10, 6-9, 6-8, 6-7, 7-20, 7-19, 7-18, 7-17, 7-16, 7-15, 7-14, 7-13, 7-12, 7-11, 7-10, 7-9, 7-8, 8-20, 8-19, 8-18, 8-17, 8-16, 8-15, 8-14, 8-13, 8-12, 8-11, 8-10, 8-9, 9-20, 9-19, 9-18, 9-17, 9-16, 9-15, 9-14, 9-13, 9-12, 9-11, 9-10, 10-20, 10-19, 10-18, 10-17, 10-16, 10-15, 10-14, 10-13, 10-12, 10-11, 11-20, 11-19, 11-18, 11-17, 11-16, 11-15, 11-14, 11-13, 11-12, 12-20, 12-19, 12-18, 12-17, 12-16, 12-15, 12-14, 12-13, 13-20, 13-19, 13-18, 13-17, 13-16, 13-15, 13-14, 14-20, 14-19, 14-18, 14-17, 14-16, 14-15, 15-20, 15-19, 15-18, 15-17, 15-16, 16-20, 16-19, 16-18, 16-17, 17-20, 17-19, 17-18, 18-20, 18-19, or 19-20 strains.

Some aspects of the disclosure relate to bacterial strains with 16S rDNA sequences that have homology or identity to a nucleic acid sequence of any one of the sequences of the bacterial strains or species described herein.

Some aspects of the disclosure relate to combinations comprising at least two (e.g., 2 or 3) of (i) a bacterial strain comprising a 16S rDNA sequence with at least 97% sequence identity to SEQ ID NO: 1, (ii) a bacterial strain comprising a 16S rDNA sequence with at least 97% sequence identity to SEQ ID NO: 2, and (iii) a bacterial strain comprising a 16S rDNA sequence with at least 97% sequence identity to SEQ ID NO: 8; and wherein the combination lacks two or more of (iv) a bacterial strain comprising a 16S rDNA sequence with at least 97% sequence identity to SEQ ID NO: 3, (v) a bacterial strain comprising a 16S rDNA sequence with at least 97% sequence identity to SEQ ID NO: 4, (vi) a bacterial strain comprising a 16S rDNA sequence with at least 97% sequence identity to SEQ ID NO: 5, (vii) a bacterial strain comprising a 16S rDNA sequence with at least 97% sequence identity to SEQ ID NO: 6, and (viii) a bacterial strain comprising a 16S rDNA sequence with at least 97% sequence identity to SEQ ID NO: 7. For example, in some embodiments, the combination comprises a bacterial strain having a 16S rDNA sequence with at least 97% sequence identity to SEQ ID NO: 1, and a bacterial strain having a 16S rDNA sequence with at least 97% sequence identity to SEQ ID NO: 2. In some embodiments, the combination comprises a bacterial strain having a 16S rDNA sequence with at least 97% sequence identity to SEQ ID NO: 1, and a bacterial strain having a 16S rDNA sequence with at least 97% sequence identity to SEQ ID NO: 8. In some embodiments, the combination comprises a bacterial strain having a 16S rDNA sequence with at least 97% sequence identity to SEQ ID NO: 2, and a bacterial strain having a 16S rDNA sequence with at least 97% sequence identity to SEQ ID NO: 8. In some embodiments, the combination comprises a bacterial strain having a 16S rDNA sequence with at least 97% sequence identity to SEQ ID NO: 1, a bacterial strain having a 16S rDNA sequence with at least 97% sequence identity to SEQ ID NO: 2, and a bacterial strain having a 16S rDNA sequence with at least 97% sequence identity to SEQ ID NO: 8.

In some embodiments, the combination does not include bacterial strains other than a bacterial strain having a 16S rDNA sequence with at least 97% sequence identity to SEQ ID NO: 1, and a bacterial strain having a 16S rDNA sequence with at least 97% sequence identity to SEQ ID NO: 2. In some embodiments, the combination does not include bacterial strains other than a bacterial strain having a 16S rDNA sequence with at least 97% sequence identity to SEQ ID NO: 1, and a bacterial strain having a 16S rDNA sequence with at least 97% sequence identity to SEQ ID NO: 8. In some embodiments, combination does not include bacterial strains other than a bacterial strain having a 16S rDNA sequence with at least 97% sequence identity to SEQ ID NO: 2, and a bacterial strain having a 16S rDNA sequence with at least 97% sequence identity to SEQ ID NO: 8. In some embodiments, the combination does not include bacterial strains other than a bacterial strain having a 16S rDNA sequence with at least 97% sequence identity to SEQ ID NO: 1, a bacterial strain having a 16S rDNA sequence with at least 97% sequence identity to SEQ ID NO: 2, and a bacterial strain having a 16S rDNA sequence with at least 97% sequence identity to SEQ ID NO: 8.

A combination comprising a bacterial strain of a species may include any number of bacterial strains belonging to that species. For example, in some embodiments, a combination comprising a bacterial strain having a 16S rDNA sequence with at least 97% sequence identity to SEQ ID NO: 1 comprises 1, 2, 3, 4, 5, 6, 7, 8, 9, 10, 11, 12, 13, 14, 15, 16, 17, 18, 19, or 20 different bacterial strains, each having a 16S rDNA sequence with at least 97% sequence identity to SEQ ID NO: 1. In some embodiments, a combination comprising a bacterial strain having a 16S rDNA sequence with at least 97% sequence identity to SEQ ID NO: 2 may comprise 1, 2, 3, 4, 5, 6, 7, 8, 9, 10, 11, 12, 13, 14, 15, 16, 17, 18, 19, or 20 bacterial strains, each having a 16S rDNA sequence with at least 97% sequence identity to SEQ ID NO: 2. In some embodiments, a combination comprising a bacterial strain having a 16S rDNA sequence with at least 97% sequence identity to SEQ ID NO: 8 may comprise 1, 2, 3, 4, 5, 6, 7, 8, 9, 10, 11, 12, 13, 14, 15, 16, 17, 18, 19, or 20 bacterial strains, each having a 16S rDNA sequence with at least 97% sequence identity to SEQ ID NO: 8. Bacterial strains with closely related 16S rDNA sequences may be distinguished using one or more of multiple features, such as colony morphology (e.g., smooth v. rough, flat v. raised, color). Bacterial strains with closely related 16S rDNA sequences may also be distinguished on the basis of presence or absence of one or more genes, such as antibiotic resistance genes, variation between sequences of additional genes, and/or sequences of whole genomes (or portions thereof).

Compositions may be said to not comprise (i.e., to lack) bacterial strains with 16S rDNA sequences related to a given sequences if bacteria having 16S rDNA sequences with the specified identity (e.g., 97%) are not detectable in the combination, e.g., above a limit of detection. Methods of detecting bacterial strains of a given species are known in the art, including culturing the bacterial strain or amplification, sequencing, and identification of 16S rDNA isolated from a biological sample (e.g., combination). In some embodiments, at least two bacterial strains, each having a 16S rDNA sequence with at least 97% sequence identity to a sequence independently selected from SEQ ID NOs: 3-7, are not present in the combination. For example, in some embodiments, the combination does not comprise a bacterial strain having a 16S rDNA sequence with at least 97% sequence identity to SEQ ID NO: 6, and does not comprise a bacterial strain with at least 97% sequence identity to SEQ ID NO: 7. In some embodiments, at least three bacterial strains, each having a 16S rDNA sequence with at least 97% sequence identity to a sequence independently selected from SEQ ID NOs: 3-7, are not present in the combination. In some embodiments, at least four bacterial strains, each having a 16S rDNA sequence with at least 97% sequence identity to a sequence independently selected from SEQ ID NOs: 3-7, are not present in the combination. In some embodiments, the combination does not comprise a bacterial strain having a 16 rDNA sequence with at least 97% sequence identity to SEQ ID NO: 3, does not comprise a bacterial strain having a 16 rDNA sequence with at least 97% sequence identity to SEQ ID NO: 4, does not comprise a bacterial strain having a 16 rDNA sequence with at least 97% sequence identity to SEQ ID NO: 5, does not comprise a bacterial strain having a 16 rDNA sequence with at least 97% sequence identity to SEQ ID NO: 6, and does not comprise a bacterial strain having a 16 rDNA sequence with at least 97% sequence identity to SEQ ID NO: 7.

Combinations described herein may include one or more additional bacterial strains. Additional bacterial strains may be taxonomically or phylogenetically related to bacterial strains having 16S rDNA sequences of SEQ ID NO: 9 or SEQ ID NO: 10. In some embodiments, one or more additional bacterial strains have 16S rDNA sequences with at least 90% sequence identity to SEQ ID NO: 9. In some embodiments, one or more additional bacterial strains have 16S rDNA sequences with at least 95% sequence identity to SEQ ID NO: 9. In some embodiments, one or more additional bacterial strains have 16S rDNA sequences with at least 97% sequence identity to SEQ ID NO: 9. In some embodiments, one or more additional bacterial strains have 16S rDNA sequences with at least 99% sequence identity to SEQ ID NO: 9. In some embodiments, one or more additional bacterial strains have 16S rDNA sequences with at least 90% sequence identity to SEQ ID NO: 10. In some embodiments, one or more additional bacterial strains have 16S rDNA sequences with at least 95% sequence identity to SEQ ID NO: 10. In some embodiments, one or more additional bacterial strains have 16S rDNA sequences with at least 97% sequence identity to SEQ ID NO: 10. In some embodiments, one or more additional bacterial strains have 16S rDNA sequences with at least 99% sequence identity to SEQ ID NO: 10.

In some embodiments, the combination comprises 2, 3, 4, 5, 6, 7, 8, 9, 10, 11, 12, 13, 14, 15, 16, 17, 18, 19, or 20 additional bacterial strains that have a 16S rDNA sequence with at least 95% sequence identity to SEQ ID NO: 9. In some embodiments, the combination comprises 2, 3, 4, 5, 6, 7, 8, 9, 10, 11, 12, 13, 14, 15, 16, 17, 18, 19, or 20 additional bacterial strains that have a 16S rDNA sequence with at least 97% sequence identity to SEQ ID NO: 9. In some embodiments, the combination comprises 2, 3, 4, 5, 6, 7, 8, 9, 10, 11, 12, 13, 14, 15, 16, 17, 18, 19, or 20 additional bacterial strains that have a 16S rDNA sequence with at least 95% sequence identity to SEQ ID NO: 10. In some embodiments, the combination comprises 2, 3, 4, 5, 6, 7, 8, 9, 10, 11, 12, 13, 14, 15, 16, 17, 18, 19, or 20 additional bacterial strains that have a 16S rDNA sequence with at least 97% sequence identity to SEQ ID NO: 10.

Some aspects of the disclosure relate to a combination comprising (i) a bacterial strain having a 16S rDNA sequence with at least 97% sequence identity to SEQ ID NO: 1, (ii) a bacterial strain having a 16S rDNA sequence with at least 97% sequence identity to SEQ ID NO: 2, (iii) a bacterial strain having a 16S rDNA sequence with at least 97% sequence identity to SEQ ID NO: 3, (iv) a bacterial strain having a 16S rDNA sequence with at least 97% sequence identity to SEQ ID NO: 4, (v) a bacterial strain having a 16S rDNA sequence with at least 97% sequence identity to SEQ ID NO: 5, (vi) a bacterial strain having a 16S rDNA sequence with at least 97% sequence identity to SEQ ID NO: 6, (vii) a bacterial strain having a 16S rDNA sequence with at least 97% sequence identity to SEQ ID NO: 7, and (viii) a bacterial strain having a 16S rDNA sequence with at least 97% sequence identity to SEQ ID NO: 8; and one or more additional bacterial strains, each having 16 rDNA sequence with at least 97% sequence identity to SEQ ID NO: 9 or SEQ ID NO: 10.

In some embodiments, the combination comprises 2, 3, 4, 5, 6, 7, 8, 9, 10, 11, 12, 13, 14, 15, 16, 17, 18, 19, or 20 additional bacterial strains, each having a 16S rDNA sequence with at least 97% sequence identity to SEQ ID NO: 9. In some embodiments, the combination comprises 2, 3, 4, 5, 6, 7, 8, 9, 10, 11, 12, 13, 14, 15, 16, 17, 18, 19, or 20 additional bacterial strains, each having a 16S rDNA sequence with at least 97% sequence identity to SEQ ID NO: 10.

In some embodiments, the combination comprises (i) a bacterial strain having a 16S rDNA sequence with at least 97% sequence identity to SEQ ID NO: 1, (ii) a bacterial strain having a 16S rDNA sequence with at least 97% sequence identity to SEQ ID NO: 2, (iii) a bacterial strain having a 16S rDNA sequence with at least 97% sequence identity to SEQ ID NO: 3, (iv) a bacterial strain having a 16S rDNA sequence with at least 97% sequence identity to SEQ ID NO: 4, (v) a bacterial strain having a 16S rDNA sequence with at least 97% sequence identity to SEQ ID NO: 5, (vi) a bacterial strain having a 16S rDNA sequence with at least 97% sequence identity to SEQ ID NO: 6, (vii) a bacterial strain having a 16S rDNA sequence with at least 97% sequence identity to SEQ ID NO: 7, and (viii) a bacterial strain having a 16S rDNA sequence with at least 97% sequence identity to SEQ ID NO: 8; and a bacterial strain having a 16S rDNA sequence with at least 97% identity to SEQ ID NO: 9.

In some embodiments, the combination comprises (i) a bacterial strain having a 16S rDNA sequence with at least 97% sequence identity to SEQ ID NO: 1, (ii) a bacterial strain having a 16S rDNA sequence with at least 97% sequence identity to SEQ ID NO: 2, (iii) a bacterial strain having a 16S rDNA sequence with at least 97% sequence identity to SEQ ID NO: 3, (iv) a bacterial strain having a 16S rDNA sequence with at least 97% sequence identity to SEQ ID NO: 4, (v) a bacterial strain having a 16S rDNA sequence with at least 97% sequence identity to SEQ ID NO: 5, (vi) a bacterial strain having a 16S rDNA sequence with at least 97% sequence identity to SEQ ID NO: 6, (vii) a bacterial strain having a 16S rDNA sequence with at least 97% sequence identity to SEQ ID NO: 7, and (viii) a bacterial strain having a 16S rDNA sequence with at least 97% sequence identity to SEQ ID NO: 8; and a bacterial strain having a 16S rDNA sequence with at least 97% identity to SEQ ID NO: 10.

In some embodiments, the combination comprises (i) a bacterial strain having a 16S rDNA sequence with at least 97% sequence identity to SEQ ID NO: 1, (ii) a bacterial strain having a 16S rDNA sequence with at least 97% sequence identity to SEQ ID NO: 2, (iii) a bacterial strain having a 16S rDNA sequence with at least 97% sequence identity to SEQ ID NO: 3, (iv) a bacterial strain having a 16S rDNA sequence with at least 97% sequence identity to SEQ ID NO: 4, (v) a bacterial strain having a 16S rDNA sequence with at least 97% sequence identity to SEQ ID NO: 5, (vi) a bacterial strain having a 16S rDNA sequence with at least 97% sequence identity to SEQ ID NO: 6, (vii) a bacterial strain having a 16S rDNA sequence with at least 97% sequence identity to SEQ ID NO: 7, and (viii) a bacterial strain having a 16S rDNA sequence with at least 97% sequence identity to SEQ ID NO: 8; a bacterial strain having a 16S rDNA sequence with at least 97% identity to SEQ ID NO: 9, and a bacterial strain having a 16S rDNA sequence with at least 97% identity to SEQ ID NO: 10.

In some embodiments, the combination does not include bacterial strains other than (i) a bacterial strain having a 16S rDNA sequence with at least 97% sequence identity to SEQ ID NO: 1, (ii) a bacterial strain having a 16S rDNA sequence with at least 97% sequence identity to SEQ ID NO: 2, (iii) a bacterial strain having a 16S rDNA sequence with at least 97% sequence identity to SEQ ID NO: 3, (iv) a bacterial strain having a 16S rDNA sequence with at least 97% sequence identity to SEQ ID NO: 4, (v) a bacterial strain having a 16S rDNA sequence with at least 97% sequence identity to SEQ ID NO: 5, (vi) a bacterial strain having a 16S rDNA sequence with at least 97% sequence identity to SEQ ID NO: 6, (vii) a bacterial strain having a 16S rDNA sequence with at least 97% sequence identity to SEQ ID NO: 7, and (viii) a bacterial strain having a 16S rDNA sequence with at least 97% sequence identity to SEQ ID NO: 8; and a bacterial strain having a 16S rDNA sequence with at least 97% identity to SEQ ID NO: 9.

In some embodiments, the combination does not include bacterial strains other than (i) a bacterial strain having a 16S rDNA sequence with at least 97% sequence identity to SEQ ID NO: 1, (ii) a bacterial strain having a 16S rDNA sequence with at least 97% sequence identity to SEQ ID NO: 2, (iii) a bacterial strain having a 16S rDNA sequence with at least 97% sequence identity to SEQ ID NO: 3, (iv) a bacterial strain having a 16S rDNA sequence with at least 97% sequence identity to SEQ ID NO: 4, (v) a bacterial strain having a 16S rDNA sequence with at least 97% sequence identity to SEQ ID NO: 5, (vi) a bacterial strain having a 16S rDNA sequence with at least 97% sequence identity to SEQ ID NO: 6, (vii) a bacterial strain having a 16S rDNA sequence with at least 97% sequence identity to SEQ ID NO: 7, and (viii) a bacterial strain having a 16S rDNA sequence with at least 97% sequence identity to SEQ ID NO: 8; and a bacterial strain having a 16S rDNA sequence with at least 97% identity to SEQ ID NO: 10.

In some embodiments, the combination does not include bacterial strains other than (i) a bacterial strain having a 16S rDNA sequence with at least 97% sequence identity to SEQ ID NO: 1, (ii) a bacterial strain having a 16S rDNA sequence with at least 97% sequence identity to SEQ ID NO: 2, (iii) a bacterial strain having a 16S rDNA sequence with at least 97% sequence identity to SEQ ID NO: 3, (iv) a bacterial strain having a 16S rDNA sequence with at least 97% sequence identity to SEQ ID NO: 4, (v) a bacterial strain having a 16S rDNA sequence with at least 97% sequence identity to SEQ ID NO: 5, (vi) a bacterial strain having a 16S rDNA sequence with at least 97% sequence identity to SEQ ID NO: 6, (vii) a bacterial strain having a 16S rDNA sequence with at least 97% sequence identity to SEQ ID NO: 7, and (viii) a bacterial strain having a 16S rDNA sequence with at least 97% sequence identity to SEQ ID NO: 8; a bacterial strain having a 16S rDNA sequence with at least 97% identity to SEQ ID NO: 9, and a bacterial strain having a 16S rDNA sequence with at least 97% identity to SEQ ID NO: 10.

A combination that includes a bacterial strain having a 16S rDNA sequence with a certain sequence identity (e.g., 97%) to a sequence (i.e., comprises a given strain, or comprises or consists of a combination of strains, at least one having 16S rDNA sequence a certain sequence identity to the reference sequence) may comprise any number of bacterial strains having the specified sequence identity to that sequence. In some embodiments, a combination that comprises a bacterial strain having at least 97% sequence identity to SEQ ID NO: 1 may comprise 2, 3, 4, 5, 6, 7, 8, 9, 10, 11, 12, 13, 14, 15, 16, 17, 18, 19, or 20 bacterial strains, each having a 16S rDNA sequence with at least 97% sequence identity to SEQ ID NO: 1.

In some embodiments, a combination that comprises a bacterial strain having at least 97% sequence identity to SEQ ID NO: 2 may comprise 2, 3, 4, 5, 6, 7, 8, 9, 10, 11, 12, 13, 14, 15, 16, 17, 18, 19, or 20 bacterial strains, each having a 16S rDNA sequence with at least 97% sequence identity to SEQ ID NO: 2.

In some embodiments, a combination that comprises a bacterial strain having at least 97% sequence identity to SEQ ID NO: 3 may comprise 2, 3, 4, 5, 6, 7, 8, 9, 10, 11, 12, 13, 14, 15, 16, 17, 18, 19, or 20 bacterial strains, each having a 16S rDNA sequence with at least 97% sequence identity to SEQ ID NO: 3.

In some embodiments, a combination that comprises a bacterial strain having at least 97% sequence identity to SEQ ID NO: 4 may comprise 2, 3, 4, 5, 6, 7, 8, 9, 10, 11, 12, 13, 14, 15, 16, 17, 18, 19, or 20 bacterial strains, each having a 16S rDNA sequence with at least 97% sequence identity to SEQ ID NO: 4.

In some embodiments, a combination that comprises a bacterial strain having at least 97% sequence identity to SEQ ID NO: 5 may comprise 2, 3, 4, 5, 6, 7, 8, 9, 10, 11, 12, 13, 14, 15, 16, 17, 18, 19, or 20 bacterial strains, each having a 16S rDNA sequence with at least 97% sequence identity to SEQ ID NO: 5.

In some embodiments, a combination that comprises a bacterial strain having at least 97% sequence identity to SEQ ID NO: 6 may comprise 2, 3, 4, 5, 6, 7, 8, 9, 10, 11, 12, 13, 14, 15, 16, 17, 18, 19, or 20 bacterial strains, each having a 16S rDNA sequence with at least 97% sequence identity to SEQ ID NO: 6.

In some embodiments, a combination that comprises a bacterial strain having at least 97% sequence identity to SEQ ID NO: 7 may comprise 2, 3, 4, 5, 6, 7, 8, 9, 10, 11, 12, 13, 14, 15, 16, 17, 18, 19, or 20 bacterial strains, each having a 16S rDNA sequence with at least 97% sequence identity to SEQ ID NO: 7.

In some embodiments, a combination that comprises a bacterial strain having at least 97% sequence identity to SEQ ID NO: 8 may comprise 2, 3, 4, 5, 6, 7, 8, 9, 10, 11, 12, 13, 14, 15, 16, 17, 18, 19, or 20 bacterial strains, each having a 16S rDNA sequence with at least 97% sequence identity to SEQ ID NO: 8.

In some embodiments, a combination that comprises a bacterial strain having at least 97% sequence identity to SEQ ID NO: 9 may comprise 2, 3, 4, 5, 6, 7, 8, 9, 10, 11, 12, 13, 14, 15, 16, 17, 18, 19, or 20 bacterial strains, each having a 16S rDNA sequence with at least 97% sequence identity to SEQ ID NO: 9.

In some embodiments, a combination that comprises a bacterial strain having at least 97% sequence identity to SEQ ID NO: 10 may comprise 2, 3, 4, 5, 6, 7, 8, 9, 10, 11, 12, 13, 14, 15, 16, 17, 18, 19, or 20 bacterial strains, each having a 16S rDNA sequence with at least 97% sequence identity to SEQ ID NO: 10.

In some embodiments, the combinations disclosed herein comprise two or more bacterial strains. In some embodiments, the combinations described herein comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or up to 20 total bacterial strains (e.g., purified bacterial strains). In some embodiments, the combinations described herein comprise 3-20, 3-19, 3-18, 3-17, 3-16, 3-15, 3-14, 3-13, 3-12, 3-11, 3-10, 3-9, 3-8, 3-7, 3-6, 3-5, 3-4, 4-20, 4-19, 4-18, 4-17, 4-16, 4-15, 4-14, 4-13, 4-12, 4-11, 4-10, 4-9, 4-8, 4-7, 4-6, 4-5, 5-20, 5-19, 5-18, 5-17, 5-16, 5-15, 5-14, 5-13, 5-12, 5-11, 5-10, 5-9, 5-8, 5-7, 5-6, 6-20, 6-19, 6-18, 6-17, 6-16, 6-15, 6-14, 6-13, 6-12, 6-11, 6-10, 6-9, 6-8, 6-7, 7-20, 7-19, 7-18, 7-17, 7-16, 7-15, 7-14, 7-13, 7-12, 7-11, 7-10, 7-9, 7-8, 8-20, 8-19, 8-18, 8-17, 8-16, 8-15, 8-14, 8-13, 8-12, 8-11, 8-10, 8-9, 9-20, 9-19, 9-18, 9-17, 9-16, 9-15, 9-14, 9-13, 9-12, 9-11, 9-10, 10-20, 10-19, 10-18, 10-17, 10-16, 10-15, 10-14, 10-13, 10-12, 10-11, 11-20, 11-19, 11-18, 11-17, 11-16, 11-15, 11-14, 11-13, 11-12, 12-20, 12-19, 12-18, 12-17, 12-16, 12-15, 12-14, 12-13, 13-20, 13-19, 13-18, 13-17, 13-16, 13-15, 13-14, 14-20, 14-19, 14-18, 14-17, 14-16, 14-15, 15-20, 15-19, 15-18, 15-17, 15-16, 16-20, 16-19, 16-18, 16-17, 17-20, 17-19, 17-18, 18-20, 18-19, or 19-20 bacterial strains. In some embodiments, the combinations comprise a mixture of bacterial strains that consists of 3-20, 3-19, 3-18, 3-17, 3-16, 3-15, 3-14, 3-13, 3-12, 3-11, 3-10, 3-9, 3-8, 3-7, 3-6, 3-5, 3-4, 4-20, 4-19, 4-18, 4-17, 4-16, 4-15, 4-14, 4-13, 4-12, 4-11, 4-10, 4-9, 4-8, 4-7, 4-6, 4-5, 5-20, 5-19, 5-18, 5-17, 5-16, 5-15, 5-14, 5-13, 5-12, 5-11, 5-10, 5-9, 5-8, 5-7, 5-6, 6-20, 6-19, 6-18, 6-17, 6-16, 6-15, 6-14, 6-13, 6-12, 6-11, 6-10, 6-9, 6-8, 6-7, 7-20, 7-19, 7-18, 7-17, 7-16, 7-15, 7-14, 7-13, 7-12, 7-11, 7-10, 7-9, 7-8, 8-20, 8-19, 8-18, 8-17, 8-16, 8-15, 8-14, 8-13, 8-12, 8-11, 8-10, 8-9, 9-20, 9-19, 9-18, 9-17, 9-16, 9-15, 9-14, 9-13, 9-12, 9-11, 9-10, 10-20, 10-19, 10-18, 10-17, 10-16, 10-15, 10-14, 10-13, 10-12, 10-11, 11-20, 11-19, 11-18, 11-17, 11-16, 11-15, 11-14, 11-13, 11-12, 12-20, 12-19, 12-18, 12-17, 12-16, 12-15, 12-14, 12-13, 13-20, 13-19, 13-18, 13-17, 13-16, 13-15, 13-14, 14-20, 14-19, 14-18, 14-17, 14-16, 14-15, 15-20, 15-19, 15-18, 15-17, 15-16, 16-20, 16-19, 16-18, 16-17, 17-20, 17-19, 17-18, 18-20, 18-19, or 19-20 strains.

TABLE 1
Examples of bacterial species of the compositions disclosed herein.

| | SEQ ID | | Closest species based | Closest species | | | |
|---|---|---|---|---|---|---|---|
| | NO. of | | on Consensus SEQ ID | based on | | | |
| | 16S | GenBank Accession | # of 16S region as | Sanger | Closest species based on | | |
| Strain | rDNA | No. of genome | compared with 16S | sequencing | WGS compared versus | Additional closely | Clostridium |
| number | sequence | sequence | database | of 16S region | WG databases | related species | cluster |
| 1 | 1 | GCA_023008325.1 | Clostridium bolteae | Clostridium | Clostridium bolteae | Lachnoclostridium | XIVa |
| | | | | bolteae | 90A9 | sp.; Enterocloster | |
| | | | | | | bolteae | |
| 2 | 2 | GCA_023008305.1 | Anaerotruncus | Anaerotruncus | Anaerotruncus | | IV |
| | | | colihominis | colihominis | colihominis DSM 17241 | | |
| 3 | 3 | GCA_023008365.1 | Sellimonas intestinalis | Sellimonas | Sellimonas intestinalis | Ruminococcus | XIVa |
| | | | | intestinalis | | torques; | |
| | | | | | | Eubacterium | |
| | | | | | | fissicatena; | |
| | | | | | | Drancourtella | |
| | | | | | | massiiliensis | |
| 4 | 4 | GCA_023015555.1 | Clostridium symbiosum | Clostridium | Clostridium symbiosum | | XIVa |
| | | | | symbiosum | WAL-14163 | | |
| 5 | 5 | GCA_023008385.1 | Blautia producta | Blautia producta | Clostridium bacterium | Blautia producta | XIVa |
| | | | | | UC5.1-1D4 | ATCC 27340; | |
| | | | | | | Blautia hominis | |
| 6 | 6 | GCA_023008345.1 | Dorea longicatena | Dorea | Dorea longicatena | | XIVa |
| | | | | longicatena | CAG:42 | | |
| 7 | 7 | GCA_023008265.1 | Clostridium innocuum | Clostridium | Erysipelotrichaceae | | XVII |
| | | | | innocuum | bacterium 21_3 | | |
| 8 | 8 | GCA_023008285.1 | Flavonifractor plautii | Flavonifractor | Clostridium orbiscindens | Subdolinogranulum | IV |
| | | | | plautii | 1_3_50AFAA | spp. | |
| 9 | 9 | | Lachnospiraceae | | Lachnospiraceae | Flavonifractor | IV |
| | | | bacterium | | bacterium 7_1_58FAA | plautii | |
| 10 | 10 | | Bifidobacterium longum | | | | N/A |

TABLE 2
Availability of bacterial strains

| | | GenBank Accession | |
|---|---|---|---|
| | Deposit | No. of 16S rDNA | |
| Species | Number | sequence | Reference |
| Clostridium | ATCC | AJ508452.1 | [No Author Listed], Int J Syst Evol Microb. |
| bolteae | BAA-613 | | 2003. 53(Pt 4):935-937 |
| Anaerotruncus | DSM 17241 | NR_027558.1 | Lawson et al., Int J Syst Evol Microb. 2004. |
| colihominis | | | 54(Pt 2): 413-417 |
| Sellimonas | DSM | NR_144722.1; | Durand et al., New Microbes New Infect. |
| intestinalis | 100357; | AM275522.1 | 2016. 11:34-42; |
| | DSM | | Hynönen et al., Anaerobe. 2016. 39:60-67; |
| | 100440 | | Muñoz et al., Microb Genom. 2020. |
| | | | 6(12):mgen000476 |
| Clostridium | ATCC | NR_118730.1 | Varel et al., Int J Syst Bacteriol. 1995. |
| symbiosum | 14940 | | 45(3):490-494 |
| Blautia producta | ATCC | NR_119217.1 | Liu et al., Int J Syst Evol Microb. 2008. |
| | 27340 | | 58(Pt 8): 1896-1902 |
| Dorea longicatena | DSM 13814 | CP102280.1 (nt | Taras et al., Int J Syst Evol Microb. 2002. |
| | | 110683-112214) | 52(Pt 2):423-428 |
| Clostridium | ATCC | M23732.2 | Weisburg et al., J Bacteriol. 1989. |
| innocuum | 14501 | | 171(12):6455-6467 |
| Flavonifractor | ATCC | CP048436.1 (nt | Carlier et al., Int J Syst Evol Microb. 2010. |
| plautii | 29863 | 1112612-114150) | 60(3): 585-590 |
| Lachnospiraceae | DSM 24814 | GCA_000242155.1 | NCBI Reference Genome-Registered |
| bacterium | | (contig JH590915.1, nt | Oct. 4, 2011 |
| | | 707-2237) | |
| Bifidobacterium | DSM 20219 | M58739.2 | Scardovi et al., Int J Syst Bacteriol. 1971. |
| longum | | | 21:276-294 |

### Sources of Bacterial Strains

In some embodiments of the compositions described herein, one or more of the bacterial strains are human-derived bacteria, meaning the one or more bacterial strains were obtained from or identified from a human or a sample therefrom (e.g., a human donor). In some embodiments, all of the bacterial strains are human-derived bacteria. In some embodiments, the bacterial strains are derived from more than one human donor.

The bacterial strains used in the compositions described herein generally are isolated from the microbiome of healthy individuals. In some embodiments, the compositions include strains originating from a single individual. In some embodiments, the compositions include strains originating from multiple individuals. In some embodiments, the bacterial strains are obtained from multiple individuals, isolated, and grown up individually. The bacterial strains that are grown up individually may subsequently be combined to provide a composition described herein. It should be appreciated that the origin of the bacterial strains of the compositions is not limited to the human microbiome from a healthy individual. In some embodiments, the bacterial strains originate from a human with a microbiome in dysbiosis. In some embodiments, the bacterial strains originate from non-human animals or the environment (e.g., soil or surface water). In some embodiments, a combination of bacterial strains described herein originates from multiple sources (e.g., human and non-human animals).

In some embodiments of the compositions described herein, the composition includes one or more anaerobic bacteria. In some embodiments of the compositions described herein, the composition includes only anaerobic bacteria. In some embodiments of the compositions described herein, the composition includes one or more facultative anaerobic bacteria. In some embodiments of the compositions described herein, the composition includes only facultative anaerobic bacteria. In some embodiments of the compositions described herein, the composition includes one or more obligate anaerobic bacteria. In some embodiments of the compositions described herein, the composition includes only obligate anaerobic bacteria.

In some embodiments of the compositions provided herein, one or more of the bacterial strains is a spore-former. In some embodiments of the compositions provided herein, one or more of the bacterial strains is in spore form. In some embodiments of the compositions provided herein, one or more of the bacterial strains is a non-spore former.

In some embodiments, the compositions described herein comprise spore forming and non-spore forming bacterial strains. In some embodiments, the compositions described herein comprise spore-forming bacterial strains. In some embodiments, the compositions described herein comprise only spore-forming bacterial strains. In some embodiments, the compositions described herein comprise only non-spore forming bacterial strains. The spore-forming bacteria can be in spore form (i.e., as spores) or in vegetative form (i.e., as vegetative cells). In spore form, bacteria are generally more resistant to environmental conditions, such as heat, acid, radiation, oxygen, chemicals, and antibiotics. In contrast, in the vegetative state or actively growing state, bacteria are more susceptible to such environmental conditions, compared to in the spore form. In general, bacterial spores are able to germinate from the spore form into a vegetative/actively growing state, under appropriate conditions. For instance, bacteria in spore format may germinate when they are introduced in the intestine.

In some embodiments, at least one (e.g., 1, 2, 3, 4, 5, 6, 7, 8, 9, or 10) of the bacterial strains in the composition is a spore-former. In some embodiments, at least one (e.g., 1, 2, 3, 4, 5, 6, 7, 8, 9, or 10) of the bacterial strains in the composition is in spore form. In some embodiments, at least one (e.g., 1, 2, 3, 4, 5, 6, 7, 8, 9, or 10) of the bacterial strains in the composition is a non-spore former. In some embodiments, at least one (e.g., 1, 2, 3, 4, 5, 6, 7, 8, 9, or 10) of the bacterial strains in the composition is in vegetative form. As discussed above, spore forming bacteria can also be in vegetative form. In some embodiments, at least one (e.g., 1, 2, 3, 4, 5, 6, 7, 8, 9, or 10) of the bacterial strains in the composition is in spore form and at least one (e.g., 1, 2, 3, 4, 5, 6, 7, 8, 9, or 10) of the bacterial strains in the composition is in vegetative form. In some embodiments, at least one bacterial strain that is considered able to form spores (i.e., a spore-former) but is present in the composition in vegetative form. In some embodiments, at least one bacterial strain that is considered able to form spores is present in the composition both in spore form and in vegetative form. In some embodiments, each of the bacterial strains is in vegetative form.

It is envisioned that the bacterial strains of the compositions described herein are alive and will be alive when they reach the target area (e.g., the intestines). Bacterial spores are considered to be alive in this regard. In some embodiments, bacteria that are administered as spores may germinate in the target area (e.g., the intestines). It should further be appreciated that not all of the bacteria are alive and the compositions can include a percentage (e.g., by weight) that is not alive. In addition, in some embodiments, the compositions include bacterial strains that are not alive when administered or at the time when the composition reaches the target area (e.g., the intestines). It is envisioned that non-living bacteria may still be useful by providing some nutrients and metabolites for the other bacterial strains in the composition.

In any of the compositions described herein, in some embodiments, the bacterial strains are purified. In any of the compositions described herein, in some embodiments, the bacterial strains are isolated. Any of the bacterial strains described herein may be isolated and/or purified, for example, from a source such as a culture or a microbiota sample (e.g., fecal matter). The bacterial strains used in the compositions described herein generally are isolated from the microbiome of healthy individuals. However, bacterial strains can also be isolated from individuals that are considered not to be healthy. In some embodiments, the compositions include strains originating from multiple individuals. As used herein, the term “isolated” with respect to bacteria refers to bacteria that have been separated from one or more undesired components, such as another bacterium or bacterial strain, one or more component of a growth medium, and/or one or more component of a sample, such as a fecal sample. In some embodiments, the bacteria are substantially isolated from a source such that other components of the source are not detected. As also used herein, the term “purified” refers to a bacterial strain or composition comprising such that has been separated from one or more components, such as contaminants. In some embodiments, the bacterial strain is substantially free of contaminants. In some embodiments, one or more bacterial strains of a composition may be independently purified from one or more other bacteria produced and/or present in a culture or a sample containing the bacterial strain. In some embodiments, a bacterial strain is isolated or purified from a sample and then cultured under the appropriate conditions for bacterial replication, e.g., under anaerobic culture conditions. The bacteria that are grown under appropriate conditions for bacterial replication can subsequently be isolated/purified from the culture in which they are grown.

### Formulation of Bacterial Strains

In some embodiments, one of or more of the bacterial strains of the compositions has been spray-dried. The process of spray-drying refers to production of a dry powder from a liquid comprising bacterial compositions. (See e.g., Ledet et al., Spray-Drying of Pharmaceuticals in “Lyophilized Biologics and Vaccines” pages 273-194, Springer). In general, the process involves rapidly drying the bacterial compositions with a hot gas. A bacterial strain may be combined with a pharmaceutical excipient prior to combining it with the other bacterial strains or multiple spray-dried bacterial strains may be combined while in spray-dried form and the mixture of bacterial strains, once combined, may be subsequently combined with a pharmaceutical excipient.

Any of the compositions described herein, including the pharmaceutical compositions and food products comprising bacterial strains, the bacterial strains in any form, for example in an aqueous form, such as a solution or a suspension, embedded in a semi-solid form, in a powdered form, or freeze-dried form. In some embodiments, the composition or the bacterial strains are lyophilized. In some embodiments, a subset of the bacterial strains is lyophilized. Suitable methods of lyophilizing compositions, specifically compositions comprising bacteria, are described, for example, in U.S. Pat. Nos. 3,261,761; 4,205,132; and PCT Publication Nos. WO 2014/029578 and WO 2012/098358, herein incorporated by reference in their entirety. The bacteria may be lyophilized as a combination and/or the bacteria may be lyophilized separately and combined prior to administration. A bacterial strain may be combined with a pharmaceutical excipient prior to combining it with the other bacterial strain or multiple lyophilized bacteria may be combined while in lyophilized form and the mixture of bacteria, once combined may subsequently be combined with a pharmaceutical excipient. In some embodiments, the bacterial strain is a lyophilized cake. In some embodiments, the compositions comprising the one or more bacterial strains are a lyophilized cake.

The bacterial strains can be manufactured using any suitable fermentation technique. In some embodiments, the bacteria are propagated or manufactured using anaerobic fermenters, which can support the rapid growth of anaerobic bacterial species. The anaerobic fermenters may be, for example, stirred tank reactors or disposable wave bioreactors. Culture media such as BL media and EG media, or similar versions of these media devoid of animal components, can be used to support the growth of the bacterial species. The bacterial product can be purified and concentrated from the fermentation broth by traditional techniques, such as centrifugation and filtration, and can optionally be dried and lyophilized by suitable methods.

### Pharmaceutical Compositions and Uses Thereof

Some aspects of the disclosure relate to a pharmaceutical composition comprising any of the bacterial combinations or bacterial strains described herein. As used herein, the term “pharmaceutical composition” refers to a composition comprising (i) at least one active ingredient, such as any one or more of the bacterial strains described herein, and (ii) one or more inactive ingredients, which may include one or more pharmaceutically acceptable excipients.

### Excipients

An “acceptable” excipient refers to an excipient that must be compatible with the active ingredient and not deleterious to the subject to which it is administered. In some embodiments, the pharmaceutically acceptable excipient is selected based on the intended route of administration of the composition, for example a composition for oral or nasal administration may comprise a different pharmaceutically acceptable excipient than a composition for rectal administration. Examples of excipients include sterile water, physiological saline, solvent, a base material, an emulsifier, a suspending agent, a surfactant, a stabilizer, a flavoring agent, an aromatic, an excipient, a vehicle, a preservative, a binder, a diluent, a tonicity adjusting agent, a soothing agent, a bulking agent, a disintegrating agent, a buffer agent, a coating agent, a lubricant, a colorant, a sweetener, a thickening agent, and a solubilizer.

Pharmaceutical compositions can be prepared in accordance with methods well known and routinely practiced in the art (see e.g., Remington: The Science and Practice of Pharmacy, Mack Publishing Co. 20th ed. 2000). The pharmaceutical compositions described herein may further comprise any carriers or stabilizers in the form of a lyophilized composition or an aqueous solution. Acceptable excipients, carriers, or stabilizers may include, for example, buffers, antioxidants, preservatives, polymers, chelating reagents, and/or surfactants. Pharmaceutical compositions are preferably manufactured under GMP conditions. The pharmaceutical compositions can be used orally, nasally or parenterally, for instance, in the form of capsules, tablets, pills, sachets, liquids, powders, granules, fine granules, film-coated preparations, pellets, troches, sublingual preparations, chewables, buccal preparations, pastes, syrups, suspensions, elixirs, emulsions, liniments, ointments, plasters, cataplasms, transdermal absorption systems, lotions, inhalations, aerosols, injections, suppositories, and the like.

### Delivery Formulations

In some embodiments, the compositions comprising bacterial strains are formulated for oral delivery. In some embodiments, the compositions comprising bacterial strains are formulated for delivery to the intestines (e.g., the small intestine and/or the colon). In some embodiments, the composition comprising bacterial strains may be formulated with an enteric coating that increases the survival of the bacteria through the harsh environment in the stomach. The enteric coating is one which resists the action of gastric juices in the stomach so that the bacteria of the composition therein will pass through the stomach and into the intestines. The enteric coating may readily dissolve when in contact with intestinal fluids, so that the bacteria enclosed in the coating will be released in the intestinal tract. Enteric coatings may consist of suitable polymers and copolymers, such as commercially available EUDRAGIT (Evonik Industries). See e.g., Zhang, AAPS PharmSciTech (2016) 17 (1), 56-67.

The compositions comprising bacterial strains may also be formulated for rectal delivery to the intestine (e.g., the colon (large intestine)). Thus, in some embodiments, compositions comprising bacterial strains may be formulated for delivery by suppository, colonoscopy, endoscopy, sigmoidoscopy or enema. A pharmaceutical preparation or formulation and particularly a pharmaceutical preparation for oral administration, may include an additional component that enables efficient delivery of the compositions of the disclosure to the intestine (e.g., the colon). A variety of pharmaceutical preparations that allow for the delivery of the compositions to the intestine (e.g., the colon) can be used. Examples thereof include pH sensitive compositions, more specifically, buffered sachet formulations or enteric polymers that release their contents when the pH becomes alkaline after the enteric polymers pass through the stomach. When a pH sensitive composition is used for formulating the pharmaceutical preparation, the pH sensitive composition is preferably a polymer whose pH threshold of the decomposition of the composition is between about 6.8 and about 7.5. Such a numeric value range is a range in which the pH shifts toward the alkaline side at a distal portion of the stomach, and hence is a suitable range for use in the delivery to the colon. It should further be appreciated that each part of the intestine (e.g., the duodenum, jejunum, ileum, cecum, colon and rectum), has different biochemical and chemical environment. For instance, parts of the intestines have different pHs, allowing for targeted delivery by compositions that have a specific pH sensitivity. Thus, the compositions provided herein may be formulated for delivery to the intestine or specific parts of the intestine (e.g., the duodenum, jejunum, ileum, cecum, colon and rectum) by providing formulations with the appropriate pH sensitivity. See e.g., Villena et al., Int J Pharm 2015, 487 (1-2): 314-9.

Also within the scope of the present disclosure are pharmaceutical compositions for administration by additional or alternative routes. In some embodiments, the pharmaceutical compositions are formulated for sublingual administration. In some embodiments, the pharmaceutical compositions are formulated for administration by injection.

In some embodiments, a pharmaceutical composition may include an additional component that enables efficient delivery of the compositions of the disclosure to a desired site, such as the gastrointestinal tract (e.g., the colon).

In some embodiments, the pharmaceutical composition includes an adjuvant associated with providing a benefit in the treatment of allergy. In some embodiments, the pharmaceutical composition includes one or more components of an oral immunotherapeutic, an epicutaneous immunotherapeutic, or a sublingual immunotherapeutic.

Another embodiment of a pharmaceutical preparation useful for delivery of the compositions to the intestine (e.g., the colon) is one that ensures the delivery to the colon by delaying the release of the contents (e.g., the bacterial strains) by approximately 3 to 5 hours, which corresponds to the small intestinal transit time. In one embodiment of a pharmaceutical preparation for delayed release, a hydrogel is used as a shell. The hydrogel is hydrated and swells upon contact with gastrointestinal fluid, with the result that the contents are effectively released (released predominantly in the colon). Delayed release dosage units include drug-containing compositions having a material which coats or selectively coats a drug or active ingredient to be administered. Examples of such a selective coating material include in vivo degradable polymers, gradually hydrolyzable polymers, gradually water-soluble polymers, and/or enzyme degradable polymers. A wide variety of coating materials for efficiently delaying the release is available and includes, for example, cellulose-based polymers such as hydroxypropyl cellulose, acrylic acid polymers and copolymers such as methacrylic acid polymers and copolymers, and vinyl polymers and copolymers such as polyvinylpyrrolidone.

Additional examples of pharmaceutical compositions that allow for the delivery to the intestine (e.g., the colon) include bioadhesive compositions which specifically adhere to the colonic mucosal membrane (for example, a polymer described in the specification of U.S. Pat. No. 6,368,586) and compositions into which a protease inhibitor is incorporated for protecting particularly a biopharmaceutical preparation in the gastrointestinal tracts from decomposition due to an activity of a protease.

Another example of a system enabling the delivery to the intestine (e.g., the colon) is a system of delivering a composition to the colon by pressure change in such a way that the contents are released by utilizing pressure change caused by generation of gas in bacterial fermentation at a distal portion of the stomach. Such a system is not particularly limited, and a more specific example thereof is a capsule which has contents dispersed in a suppository base and which is coated with a hydrophobic polymer (for example, ethyl cellulose).

A further example of a system enabling the delivery of a composition to the intestine (e.g., the colon), is a composition that includes a coating that can be removed by an enzyme present in the gut (e.g., the colon), such as, for example, a carbohydrate hydrolase or a carbohydrate reductase. Such a system is not particularly limited, and more specific examples thereof include systems which use food components such as non-starch polysaccharides, amylose, xanthan gum, and azopolymers.

The compositions provided herein can also be delivered to specific target areas, such as the intestine, by delivery through an orifice (e.g., a nasal tube) or through surgery. In addition, the compositions provided herein that are formulated for delivery to a specific area (e.g., the cecum or the colon), may be administered by a tube (e.g., directly into the small intestine). Combining mechanical delivery methods such as tubes with chemical delivery methods such as pH specific coatings, allow for the delivery of the compositions provided herein to a desired target area (e.g., the cecum or the colon).

The pharmaceutical compositions comprising bacterial strains are in pharmaceutically acceptable dosage forms by conventional methods known to those of skill in the art. Dosage regimens are adjusted to provide the optimum desired response (e.g., the prophylactic or therapeutic effect). In some embodiments, the dosage form of the composition is a tablet, pill, capsule, powder, granules, solution, or suppository. In some embodiments, the pharmaceutical composition is formulated for oral administration. In some embodiments, the pharmaceutical composition comprises bacterial strains and is formulated such that the bacteria, or a portion thereof, remain viable after passage through the stomach of the subject. In some embodiments, the pharmaceutical composition is formulated for rectal administration, e.g., as a suppository. In some embodiments, the pharmaceutical composition is formulated for delivery to the intestine or a specific area of the intestine (e.g., the colon) by providing an appropriate coating (e.g., a pH specific coating, a coating that can be degraded by target area specific enzymes, or a coating that can bind to receptors that are present in a target area).

### Uses

Some aspects of the disclosure relate to methods comprising administering a composition comprising two or more bacterial strains. For example, a composition (e.g., pharmaceutical composition) or food product as described herein may be administered to a subject. The composition may be administered in an amount sufficient for one or more bacterial strains of the composition to colonize the microbiome of a subject. In some embodiments, the composition is administered in a therapeutically effective amount for treating a pathogenic infection in a subject. In some embodiments, the composition is administered in a therapeutically effective amount for preventing a pathogenic infection in a subject. In some embodiments, the method further comprises administering an antibiotic to the subject prior to administration of the composition. In some embodiments, the method further comprises administering vancomycin to the subject prior to administration of any of the compositions described herein.

In some embodiments, the two or more of the bacterial strains of the compositions provided herein colonize or recolonize the gastrointestinal tract or parts thereof (e.g., the colon or the cecum) of the subject. Such colonization may also be referred to as grafting or engraftment. In some embodiments, two or more of the bacterial strains of the compositions recolonize the intestinal tract (e.g., the colon or the cecum) of the subject after the naturally present microbiome has been partially or completely removed, e.g., due to administration of an antibiotic. In some embodiments, the two or more of the bacterial strains of the compositions recolonize the intestinal tract (e.g., the colon or the cecum) of the subject after the naturally present microbiome has been partially or completely removed by antibiotic (e.g., vancomycin) treatment. In some embodiments, the two or more of the bacterial strains of the compositions colonize a dysbiotic gastrointestinal tract (e.g., a gastrointestinal tract that has undergone antibiotic treatment). In some embodiments, all of the bacterial strains of the composition colonize the gastrointestinal tract. In some embodiments, at least one (e.g., 1, 2, 3, 4, 5, 6, 7, 8, 9, 10, 11, 12, 13, 14, 15, or 16) of the bacterial strains of the compositions colonize the gastrointestinal tract. In some embodiments, all of the bacterial strains of the compositions colonize a dysbiotic gastrointestinal tract. In some embodiments, multiple doses of the composition are administered to allow for all of the bacterial strains of the composition colonize the gastrointestinal tract. In some embodiments, multiple doses of the composition are administered to allow for all of the bacterial strains of the compositions colonize a dysbiotic gastrointestinal tract. In some embodiments, colonization of the microbiome using the compositions and methods provide for an increase in the abundance of bacterial species beneficial to the microbiome.

In some embodiments, colonization of a microbiome with the bacterial strains of the compositions described herein results in a healthy microbiome. As used herein, a “healthy microbiome,” refers to a microbiome from a subject who does not have overt disease (e.g., a healthy subject). Although the microbial composition of healthy microbiomes can vary widely, several trends have emerged which characterize healthy microbiomes. For example, the gastrointestinal microbiome may perform a number of metabolic and/or other molecular functions, including the metabolism of carbohydrates, lipids, and other nutrients which are performed by healthy microbiomes, regardless of the specific species composition. In some instances, the metabolic and molecular functions carried out by a healthy microbiome cannot be performed by the host subject, resulting in a symbiotic host-microbial relationship. Additionally, healthy microbiomes tend to be resilient to external (e.g., dietary or pharmaceutical) and/or internal (e.g., age, disease-state, stress, inflammation) changes in the subject. The resilience of a healthy microbiome can also be characterized by the ability and the rate at which a healthy state is restored after occurrence of a perturbation. Alternatively, or in addition, a healthy microbiome may be characterized by a high (e.g., greater than 75%) relative abundance of bacterial species from the phylum Firmicutes and genus Bacteroides relative to species from the phylum Proteobacteria.

In some embodiments, administration of the compositions described herein reduces the risk of C. difficile infection (CDI) in a subject. In some embodiments, administration of the compositions described herein prevents CDI in a subject. In some embodiments, administration of the compositions reduces the risk of recurrent C. difficile infection (rCDI) in a subject. rCDI is CDI that occurs more than once in the same subject. rCDI is associated with reduced abundance of SCFAs, increased abundance of primary bile acids, and reduced abundance of secondary bile acids. In some embodiments, administration of the compositions described herein prevents rCDI in a subject.

In some embodiments, administration of the compositions described herein results in a decrease in the abundance of microorganisms associated with recurrence of C. difficile infection. Microorganisms associated with recurrence of C. difficile infection are described herein in Example 1 and include, for example, Lactobacillus harbinensis, Campylobacter concisus, Lactobacillus rhamnosus, Clostridia bacterium UC5.1.1F8, Veillonella sp. oral taxon 158, Lactobacillus paracasei, Blautia massiliensis, Bacteroides caccae CAG.21, Enterobacter cloacae. Additional microorganisms associated with recurrence of C. difficile infection include those of the genera Campylobacter, Kluyvera, and Veillonella, those of the family Campylobacteraceae, those of the order Campylobacterales, and those of the class Epsilonproteobacteria.

In some embodiments, administration of the compositions described herein results in a decrease in the abundance of Proteobacteria. In some embodiments, administration of the compositions described herein results in a decrease in the abundance of one or more bacterial strain that belongs to the phylum Proteobacteria. Proteobacteria is a phylum of Gram-negative bacteria that includes a number of pathogens, such as Escherichia coli, Klebsiella sp., Salmonella sp., Campylobacter sp., and Pseudomonas sp. In some embodiments, decreasing dysbiosis results in a decrease in the abundance of one or more bacterial species belonging to the phylum Proteobacteria. In some embodiments, decreasing dysbiosis results in a decrease in the abundance overall of bacterial species belonging to the phylum Proteobacteria.

In some embodiments, administration of the compositions described herein results in a decrease in abundance of Proteobacteria in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Proteobacteria in the subject (or microbiome thereof) prior to administering the compositions. In some embodiments, the abundance of Proteobacteria in the subject prior to the administration of the compositions was higher because of treatment with an antibiotic. In some embodiments, the abundance of Proteobacteria in the subject prior to the administration of the compositions was higher because of treatment with an antibiotic. In some embodiments, administration of the compositions described herein results in a decrease in abundance of Proteobacteria in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Proteobacteria in another subject (e.g., a reference subject) (or microbiome thereof) who did not receive the compositions.

In some embodiments, administration of the compositions described herein results in a decrease in abundance of Proteobacteria in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the abundance of Proteobacteria in the subject (or microbiome thereof) prior to administering the compositions. In some embodiments, administration of the compositions described herein results in decrease in the abundance of Proteobacteria in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the abundance of Proteobacteria in a subject (e.g., a reference subject) (or microbiome thereof) who did not receive the compositions.

In some embodiments, administration of the compositions described herein results in a decrease in abundance of Epsilonproteobacteria in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Epsilonproteobacteria in the subject (or microbiome thereof) prior to administering the compositions. In some embodiments, the abundance of Epsilonproteobacteria in the subject prior to the administration of the compositions was higher because of treatment with an antibiotic. In some embodiments, the abundance of Epsilonproteobacteria in the subject prior to the administration of the compositions was higher because of treatment with an antibiotic. In some embodiments, administration of the compositions described herein results in a decrease in abundance of Epsilonproteobacteria in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Epsilonproteobacteria in another subject (e.g., a reference subject) (or microbiome thereof) who did not receive the compositions.

In some embodiments, administration of the compositions described herein results in a decrease in abundance of Epsilonproteobacteria in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the abundance of Epsilonproteobacteria in the subject (or microbiome thereof) prior to administering the compositions. In some embodiments, administration of the compositions described herein results in decrease in the abundance of Epsilonproteobacteria in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the abundance of Epsilonproteobacteria in a subject (e.g., a reference subject) (or microbiome thereof) who did not receive the compositions.

In some embodiments, administration of the compositions described herein results in a decrease in abundance of Gammaproteobacteria subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Gammaproteobacteria in the subject (or microbiome thereof) prior to administering the compositions. In some embodiments, the abundance of Gammaproteobacteria in the subject prior to the administration of the compositions was higher because of treatment with an antibiotic. In some embodiments, the abundance of Gammaproteobacteria in the subject prior to the administration of the compositions was higher because of treatment with an antibiotic. In some embodiments, administration of the compositions described herein results in a decrease in abundance of Gammaproteobacteria in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Gammaproteobacteria in another subject (e.g., a reference subject) (or microbiome thereof) who did not receive the compositions.

In some embodiments, administration of the compositions described herein results in a decrease in abundance of Gammaproteobacteria in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the abundance of Gammaproteobacteria in the subject (or microbiome thereof) prior to administering the compositions. In some embodiments, administration of the compositions described herein results in decrease in the abundance of Gammaproteobacteria in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the abundance of Gammaproteobacteria in a subject (e.g., a reference subject) (or microbiome thereof) who did not receive the compositions.

In some embodiments, administration of the compositions described herein results in a decrease in abundance of Campylobacterales in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Campylobacterales in the subject (or microbiome thereof) prior to administering the compositions. In some embodiments, the abundance of Campylobacterales in the subject prior to the administration of the compositions was higher because of treatment with an antibiotic. In some embodiments, the abundance of Campylobacterales in the subject prior to the administration of the compositions was higher because of treatment with an antibiotic. In some embodiments, administration of the compositions described herein results in a decrease in abundance of Campylobacterales in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Campylobacterales in another subject (e.g., a reference subject) (or microbiome thereof) who did not receive the compositions.

In some embodiments, administration of the compositions described herein results in a decrease in abundance of Campylobacterales in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the abundance of 5 Campylobacterales in the subject (or microbiome thereof) prior to administering the compositions. In some embodiments, administration of the compositions described herein results in decrease in the abundance of Campylobacterales in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the abundance of Campylobacterales in a subject (e.g., a reference subject) (or microbiome thereof) who did not receive the compositions.

In some embodiments, administration of the compositions described herein results in a decrease in abundance of Bacilli in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Bacilli in the subject (or microbiome thereof) prior to administering the compositions. In some embodiments, the abundance of Bacilli in the subject prior to the administration of the compositions was higher because of treatment with an antibiotic. In some embodiments, the abundance of Bacilli in the subject prior to the administration of the compositions was higher because of treatment with an antibiotic. In some embodiments, administration of the compositions described herein results in a decrease in abundance of Bacilli in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Bacilli in another subject (e.g., a reference subject) (or microbiome thereof) who did not receive the compositions.

In some embodiments, administration of the compositions described herein results in a decrease in abundance of Bacilli in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the abundance of Bacilli in the subject (or microbiome thereof) prior to administering the compositions. In some embodiments, administration of the compositions described herein results in decrease in the abundance of Bacilli in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the abundance of Bacilli in a subject (e.g., a reference subject) (or microbiome thereof) who did not receive the compositions.

In some embodiments, administration of the compositions described herein results in a decrease in abundance of Campylobacteraceae in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Campylobacteraceae in the subject (or microbiome thereof) prior to administering the compositions. In some embodiments, the abundance of Campylobacteraceae in the subject prior to the administration of the compositions was higher because of treatment with an antibiotic. In some embodiments, the abundance of Campylobacteraceae in the subject prior to the administration of the compositions was higher because of treatment with an antibiotic. In some embodiments, administration of the compositions described herein results in a decrease in abundance of Campylobacteraceae in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Campylobacteraceae in another subject (e.g., a reference subject) (or microbiome thereof) who did not receive the compositions.

In some embodiments, administration of the compositions described herein results in a decrease in abundance of Campylobacteraceae in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the abundance of Campylobacteraceae in the subject (or microbiome thereof) prior to administering the compositions. In some embodiments, administration of the compositions described herein results in decrease in the abundance of Campylobacteraceae in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the abundance of Campylobacteraceae in a subject (e.g., a reference subject) (or microbiome thereof) who did not receive the compositions.

In some embodiments, administration of the compositions described herein results in a decrease in abundance of Campylobacter, Klebsiella, Kluyvera, or Veillonella in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Campylobacter, Klebsiella, Kluyvera, or Veillonella in the subject (or microbiome thereof) prior to administering the compositions. In some embodiments, the abundance of Campylobacter, Klebsiella, Kluyvera, or Veillonella in the subject prior to the administration of the compositions was higher because of treatment with an antibiotic. In some embodiments, the abundance of Campylobacter, Klebsiella, Kluyvera, or Veillonella in the subject prior to the administration of the compositions was higher because of treatment with an antibiotic. In some embodiments, administration of the compositions described herein results in a decrease in abundance of Campylobacter, Klebsiella, Kluyvera, or Veillonella in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Campylobacter, Klebsiella, Kluyvera, or Veillonella in another subject (e.g., a reference subject) (or microbiome thereof) who did not receive the compositions.

In some embodiments, administration of the compositions described herein results in a decrease in abundance of Campylobacter, Klebsiella, Kluyvera, or Veillonella in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the abundance of Campylobacter, Klebsiella, Kluyvera, or Veillonella in the subject (or microbiome thereof) prior to administering the compositions. In some embodiments, administration of the compositions described herein results in decrease in the abundance of Campylobacter, Klebsiella, Kluyvera, or Veillonella in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the abundance of Campylobacter, Klebsiella, Kluyvera, or Veillonella in a subject (e.g., a reference subject) (or microbiome thereof) who did not receive the compositions.

In some embodiments, administration of the compositions described herein results in a decrease in abundance of Enterococcus in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Enterococcus in the subject (or microbiome thereof) prior to administering the compositions. In some embodiments, the abundance of Enterococcus in the subject prior to the administration of the compositions was higher because of treatment with an antibiotic. In some embodiments, the abundance of Enterococcus in the subject prior to the administration of the compositions was higher because of treatment with an antibiotic. In some embodiments, administration of the compositions described herein results in a decrease in abundance of Enterococcus in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Enterococcus in another subject (e.g., a reference subject) (or microbiome thereof) who did not receive the compositions.

In some embodiments, administration of the compositions described herein results in a decrease in abundance of Enterococcus in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the abundance of Enterococcus in the subject (or microbiome thereof) prior to administering the compositions. In some embodiments, administration of the compositions described herein results in decrease in the abundance of Enterococcus in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the abundance of Enterococcus in a subject (e.g., a reference subject) (or microbiome thereof) who did not receive the compositions.

In some embodiments, administration of the compositions described herein results in a decrease in abundance of Lactobacillus harbinensis, Campylobacter concisus, Lactobacillus rhamnosus, Clostridia bacterium UC5.1.1F8, Veillonella sp. oral taxon 158, Lactobacillus paracasei, Blautia massiliensis, Bacteroides caccae CAG.21, or Enterobacter cloacae in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Lactobacillus harbinensis, Campylobacter concisus, Lactobacillus rhamnosus, Clostridia bacterium UC5.1.1F8, Veillonella sp. oral taxon 158, Lactobacillus paracasei, Blautia massiliensis, Bacteroides caccae CAG.21, or Enterobacter cloacae in the subject (or microbiome thereof) prior to administering the compositions. In some embodiments, the abundance of Lactobacillus harbinensis, Campylobacter concisus, Lactobacillus rhamnosus, Clostridia bacterium UC5.1.1F8, Veillonella sp. oral taxon 158, Lactobacillus paracasei, Blautia massiliensis, Bacteroides caccae CAG.21, or Enterobacter cloacae in the subject prior to the administration of the compositions was higher because of treatment with an antibiotic. In some embodiments, the abundance of Lactobacillus harbinensis, Campylobacter concisus, Lactobacillus rhamnosus, Clostridia bacterium UC5.1.1F8, Veillonella sp. oral taxon 158, Lactobacillus paracasei, Blautia massiliensis, Bacteroides caccae CAG.21, or Enterobacter cloacae in the subject prior to the administration of the compositions was higher because of treatment with an antibiotic. In some embodiments, administration of the compositions described herein results in a decrease in abundance of Lactobacillus harbinensis, Campylobacter concisus, Lactobacillus rhamnosus, Clostridia bacterium UC5.1.1F8, Veillonella sp. oral taxon 158, Lactobacillus paracasei, Blautia massiliensis, Bacteroides caccae CAG.21, or Enterobacter cloacae in the subject (or microbiome thereof) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the abundance of Lactobacillus harbinensis, Campylobacter concisus, Lactobacillus rhamnosus, Clostridia bacterium UC5.1.1F8, Veillonella sp. oral taxon 158, Lactobacillus paracasei, Blautia massiliensis, Bacteroides caccae CAG.21, or Enterobacter cloacae in another subject (e.g., a reference subject) (or microbiome thereof) who did not receive the compositions.

In some embodiments, administration of the compositions described herein results in a decrease in abundance of Lactobacillus harbinensis, Campylobacter concisus, Lactobacillus rhamnosus, Clostridia bacterium UC5.1.1F8, Veillonella sp. oral taxon 158, Lactobacillus paracasei, Blautia massiliensis, Bacteroides caccae CAG.21, or Enterobacter cloacae in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%, as compared to the abundance of Lactobacillus harbinensis, Campylobacter concisus, Lactobacillus rhamnosus, Clostridia bacterium UC5.1.1F8, Veillonella sp. oral taxon 158, Lactobacillus paracasei, Blautia massiliensis, Bacteroides caccae CAG.21, or Enterobacter cloacae in the subject (or microbiome thereof) prior to administering the compositions. In some embodiments, administration of the compositions described herein results in decrease in the abundance of Lactobacillus harbinensis, Campylobacter concisus, Lactobacillus rhamnosus, Clostridia bacterium UC5.1.1F8, Veillonella sp. oral taxon 158, Lactobacillus paracasei, Blautia massiliensis, Bacteroides caccae CAG.21, or Enterobacter cloacae in the subject (or microbiome thereof) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the abundance of Lactobacillus harbinensis, Campylobacter concisus, Lactobacillus rhamnosus, Clostridia bacterium UC5.1.1F8, Veillonella sp. oral taxon 158, Lactobacillus paracasei, Blautia massiliensis, Bacteroides caccae CAG.21, or Enterobacter cloacae in a subject (e.g., a reference subject) (or microbiome thereof) who did not receive the compositions.

The abundance of bacteria, including the abundance of specific species or strains of bacteria and abundance of a population of bacteria (e.g., bacteria belonging to a particular phylum) may be assessed using any suitable method. In general, the abundance of bacteria may be assessed directly or indirectly. Examples of methods for directly assessing the abundance of bacteria in a sample (e.g., a microbiome or sample thereof) include identifying and quantifying bacterial strains in a fecal sample from the subject. Examples of methods for indirectly assessing the abundance of bacteria in a sample (e.g., a microbiome or sample thereof) include sequencing of nucleic acid samples (e.g., 16S rRNA gene for a given bacterial species or other bacterial genes) obtained from a fecal or biopsy sample and detecting and quantifying metabolites associated with specific bacteria (e.g., phospholipid fatty acid metabolism, microbial biomass carbon analysis) in a fecal sample from the subject.

In some embodiments, the one or more bacterial strains of the compositions colonize the microbiome because they can “outgrow” other bacterial strains (e.g., pathogens such as C. difficile). In some embodiments, the subject has been treated with an antibiotic resulting in a removal of most of the microbiome, providing a “clean slate” environment for both the one or more bacterial strains of compositions and any other bacterial strains (e.g., pathogens, strains associated with inflammation or undesired immune responses). Thus, without being limited to a specific mechanism, if a pathogen and bacterial strains of the compositions provided herein are both present in the intestinal tract (e.g., the colon or the cecum), the bacterial strains of compositions provided herein grow faster (e.g., have a shorter doubling time) than the pathogen, thereby preventing the pathogen from accumulating in the intestinal tract (e.g., the colon or the cecum) and allowing the bacterial strains of the compositions to colonize. In some embodiments, the faster growth results because the bacterial strains of the compositions provided herein are better at grafting in the intestinal tract (e.g., the colon or the cecum). In some embodiments, the faster growth results because the bacterial strains of the compositions provided herein are better at metabolizing nutrients present in the intestinal tract (e.g., the colon or the cecum). In some embodiments, the compositions of bacterial strains provided herein prevent or inhibit production of bacterial toxins by an infectious agent or prevent or inhibit the cytopathic or cytotoxic effects of such toxins. In some embodiments, the bacterial strains of the compositions provided herein can treat pathogenic infections, because of the synergy between the bacterial strains. Thus, without being limiting, in some embodiments, the combination of the bacterial strains of the compositions provided herein act synergistically because the combination of the strains is particularly well-suited to use nutrients in the intestinal tract (e.g., the colon or the cecum), for instance through metabolic interactions, and/or because the combination is superior in grafting (e.g., by providing a favorable microenvironment). In some embodiments, the bacterial strains of the compositions described herein are able to colonize specific niches in the intestinal tract (e.g., the colon or the cecum). In some embodiments, the bacterial strains of the compositions described herein are able to colonize specific niches in the intestinal tract (e.g., the colon or the cecum) that became available after antibiotic treatment.

In some embodiments, at least 1, at least 2, at least 3, at least 4, at least 5, at least 6, at least 7, at least 8, at least 9, or at least 10 of the bacterial strains of the composition colonize the microbiome of the subject. In some embodiments, 1-10, 2-10, 3-10, 4-10, 5-10, 6-10, 7-10, 8-10, 9-10, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2 of the bacterial strains of the composition colonize the microbiome of the subject. In some embodiments, 1 to 3 of the bacterial strains of the composition colonize the microbiome of the subject. In some embodiments, 1 to 8 of the bacterial strains of the composition colonize the microbiome of the subject. In some embodiments, 3 to 8 of the bacterial strains of the composition colonize the microbiome of the subject. In some embodiments, 3 to 10 of the bacterial strains of the composition colonize the microbiome of the subject. In some embodiments, 3 of the bacterial strains colonize the microbiome of the subject. In some embodiments, Clostridium bolteae, Anaerotruncus colihominis, and Flavonifractor plautii colonize the microbiome of the subject. In some embodiments, at least two of Clostridium bolteae, Anaerotruncus colihominis, and Flavonifractor plautii colonize the microbiome of the subject. In some embodiments, Bifidobacterium longum and/or Lachnospiraceae bacterium colonize the microbiome of the subject. In some embodiments, Bifidobacterium longum and Lachnospiraceae bacterium colonize the microbiome of the subject.

The extent of colonization of any of the bacterial strains may be determined, for example by detecting the presence of one or more bacterial strains and/or by quantifying the abundance of the one or more bacterial strains. In some embodiments, at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the bacterial strains of the compositions colonize the microbiome of the subject. In some embodiments, at least 25% of the bacterial strains of the compositions colonize the microbiome of the subject. In some embodiments, at least 50% of the bacterial strains of the compositions colonize the microbiome of the subject. In some embodiments, 100% of the bacterial strains of the compositions colonize the microbiome of the subject. In some embodiments, the percentage of the bacterial strains of the compositions that colonize the microbiome of the subject is increased by administering additional doses of the compositions.

The methods described herein involve administering any of the compositions (e.g., pharmaceutical compositions) described herein to a subject in need thereof. As used herein, “subject,” “individual,” and “patient” are used interchangeably, and refer to a vertebrate, preferably a mammal such as a human. Mammals include, but are not limited to, human primates, non-human primates or murine, bovine, equine, canine or feline species. In some embodiments, the subject is a human. In some embodiments, the human subject is a neonatal subject, a pediatric subject, an adolescent subject, an adult subject, or a geriatric subject.

Any of the compositions described herein may be administered to a subject in a therapeutically effective amount or a dose of a therapeutically effective amount. In some embodiments, the therapeutically effective amount is an amount sufficient to treat or prevent a disease or disorder. The terms “treat” or “treatment” refer to reducing or alleviating one or more of the symptoms associated with a disease (e.g., pathogenic infection, such as C. difficile infection). The terms “prevent” or “prevention” encompass prophylactic administration and may reduce the incidence or likelihood of pathogenic infection or a recurrent or chronic pathogenic infection. For instance, in some embodiments, administration of any of the compositions provided herein results in a healthy microbiome in the subject that provides an effect in a subject that reduces the incidence or likelihood of a disease or disorder. In some embodiments, administration of any of the compositions provided herein results in a reduction or alleviation of one or more symptom associated with disease or disorder.

As used herein, the term “therapeutically effective amount” may be used interchangeably with the term “effective amount.” A therapeutically effective amount or an effective amount of a composition, such as a pharmaceutical composition, as described herein, is any amount that results in a desired response or outcome in a subject, such as those described herein, including but not limited to delay the manifestation, arrest the progression, relieve or alleviate at least one symptom of a disease or disorder (e.g., C. difficile infection or recurrent infection), reduce the incidence or likelihood of the occurrence of a disease or disorder (e.g., C. difficile infection or recurrent infection), reduces the risk of a disease or disorder (e.g., C. difficile infection or recurrent infection), and/or colonization of the microbiome by bacterial strains of the compositions described herein. In some embodiments, the therapeutically effective amount is an amount sufficient to colonize the microbiome of a subject. In some embodiments, the therapeutically effective amount is an amount sufficient to restore the microbiome following a state of disease. In some embodiments, the therapeutically effective amount is an amount sufficient to reduce or eliminate at least one symptom associated with pathogenic infection or reduce the severity of at least one symptom associated with pathogenic infection. In some embodiments, the therapeutically effective amount is an amount sufficient to reduce or eliminate at least one symptom associated with pathogenic infection or reduce the severity of at least one symptom associated with pathogenic infection.

It should be appreciated that the term “effective amount,” in reference to a composition comprising bacterial strains, may be expressed as the number of bacteria or CFUs to be administered. It should further be appreciated that the bacteria can multiply once administered. Thus, administration of even a relatively small amount of bacteria may have therapeutic effects.

In some embodiments, the disease or disorder is a pathogenic infection. In some embodiment, the pathogenic infection is an infection with Clostridium difficile, Klebsiella, Kluyvera, Veillonella, Enterococci, Enterobacteriaceae, Neisseria gonorrhoeae, Acinetobacter, Campylobacter sp., Pseudomonas aeruginosa, Salmonella sp., Shigella sp., Staphylococcus aureus, Salmonella typhi, Group A Streptococcus, Group B Streptococcus, or Streptococcus pneumoniae. In some embodiments, the pathogenic infection is a C. difficile infection. In some embodiments, the pathogenic infection is a recurrent C. difficile infection. In some embodiments, the pathogenic infection is a Klebsiella infection. In some embodiments, the pathogenic infection is a Kluyvera infection. In some embodiments, the pathogenic infection is a Veillonella infection.

In some aspects, the compositions and methods provided herein alleviate and/or reduce the likelihood of recurrent C. difficile infection in a subject. In some embodiments, the likelihood of recurrent C. difficile infection is assessed over a specific period of time. In some embodiments, the likelihood of recurrent C. difficile infection is assessed over 1, 2, 3, 4, 5, 6, 7, 8, 9, 10, 11, 12, 13, 14, 15, 16, 17, 18, 19, 20, 21, 22, 23, 24, 25, or 26 weeks. In some embodiments, the likelihood of recurrent C. difficile infection is assessed over 1, 2, 3, 4, 5, 6, 7, 8, 9, 10, 11, or 12 months. In some embodiments, the likelihood of recurrent C. difficile infection is assessed over 1, 2, 3, 4, 5, 6, 7, 8, 9, 10, 11, 12, 13, 14, 15, 16, 17, 18, 19, or 20 years.

In some aspects, the compositions and methods provided herein alleviate and/or reduce the incidence of recurrent C. difficile infection in a subject. In some embodiments, the incidence of recurrent C. difficile infection is assessed over a specific period of time. In some embodiments, the incidence of recurrent C. difficile infection is assessed over 1, 2, 3, 4, 5, 6, 7, 8, 9, 10, 11, 12, 13, 14, 15, 16, 17, 18, 19, 20, 21, 22, 23, 24, 25, or 26 weeks. In some embodiments, the incidence of recurrent C. difficile infection is assessed over 1, 2, 3, 4, 5, 6, 7, 8, 9, 10, 11, or 12 months. In some embodiments, the incidence of recurrent C. difficile infection is assessed over 1, 2, 3, 4, 5, 6, 7, 8, 9, 10, 11, 12, 13, 14, 15, 16, 17, 18, 19, or 20 years.

In some embodiments, the therapeutically effective amount is an amount sufficient to reduce the incidence or likelihood of severe recurrent C. difficile infection. In some embodiments, the therapeutically effective amount is an amount sufficient to reduce the incidence or likelihood of a life-threatening recurrent C. difficile infection. In some embodiments, the therapeutically effective amount is an amount sufficient to reduce the incidence or likelihood of a fulminant recurrent C. difficile infection. Severe infections are characterized by elevated white blood cell counts (at least 15,000 cells/mL blood) and reduced kidney function (serum creatinine >1.5 mg/dL). Fulminant infections are characterized by shock, hypotension, ileus, and/or toxic megacolon.

In some embodiments, the specific combination of one or more bacterial strains of the compositions described herein provides a synergistic effect that promotes treating and/or preventing pathogen infection and/or reducing the risk and/or occurrence of pathogen infection. In some embodiments, the specific combination of one or more bacterial strains of the compositions described herein provides a synergistic effect that promotes treating and/or preventing C. difficile infection and/or reducing the risk and/or recurrence of C. difficile infection. In some embodiments, the specific combination of one or more bacterial strains of the compositions described herein provides a synergistic effect that promotes treating and/or preventing C. difficile infection in a subject. In some embodiments, the synergistic effect is provided by the capacity of the combination to metabolize specific nutrients. In some embodiments, the synergistic effect is provided by the capacity of the combination to provide specific metabolites to the environment. Such specific metabolites may suppress growth of the pathogen and/or stimulate growth of non-pathogens.

In some embodiments, the synergistic effect is provided by the capacity of the combination to provide short-chain fatty acids to the environment. In some embodiments, the synergistic effect is provided by the capacity of the combination to provide specific short-chain fatty acids to the environment. In some embodiments, the synergistic effect is provided by the capacity of the combination to produce butyrate. In some embodiments, the synergistic effect is provided by the capacity of the combination to produce acetate. In some embodiments, the synergistic effect is provided by the capacity of the combination to produce lactate. In some embodiments, the synergistic effect is provided by the capacity of the combination to produce propionate. In some embodiments, the synergistic effect is provided by the capacity of the combination to produce succinate. In some embodiments, the synergistic effect is provided by the capacity of the combination to produce multiple metabolites. In some embodiments, the synergistic effect is provided by the capacity of the combination to produce multiple short-chain fatty acids. In some embodiments, the synergistic effect is provided by the capacity of the combination to produce both butyrate and acetate. In some embodiments, the synergistic effect is provided by the capacity of the combination to produce both butyrate and lactate. In some embodiments, the synergistic effect is provided by the capacity of the combination to produce both butyrate and propionate. In some embodiments, the synergistic effect is provided by the capacity of the combination to produce both butyrate and succinate. In some embodiments, the synergistic effect is provided by the capacity of the combination to produce butyrate, acetate, and additional short-chain fatty acids.

In some embodiments, the specific combination of two or more bacterial strains of the compositions provided herein is superior in the use of nutrients and in grafting when compared to other strains (e.g., pathogens), thereby protecting and or restoring the microbiome, for instance through suppressing the growth of the pathogen. In some embodiments, the specific combination of one or more bacterial strains of the compositions provided herein induces an immune response in the subject that promotes colonizing the microbiome of the subject with one of or of the bacterial strains of the compositions. In some embodiments, the specific combination of one or more bacterial strains of the compositions provided herein induces an immune response in the subject that reduces symptoms of C. difficile infection or risk of C. difficile infection recurrence.

Some aspects of the compositions and methods described herein decrease the abundance of primary bile acids, increase the abundance of secondary bile acids, and/or promote conversion of primary bile acids into secondary bile acids. Bile acids are steroid acids that allow the digestion of dietary fats and oils by acting as surfactants that turn the fats and oils into micelles. Bile acids also act as hormones utilizing the farnesoid X receptor and GBPAR1. Primary bile acids are synthesized in the liver from cholesterol and a conjugated with either taurine or glycine prior to secretion. When the primary bile acids are secreted into the lumen of the intestine, bacteria partially dehydroxylate and remove the glycine or taurine groups, forming secondary bile acids.

Non-limiting examples of primary bile acids are cholic acid (CA), chenodeoxycholic acid (CDCA), glycocholic acid (GCA), glycochenodeoxycholic acid (GCDCA), glycodeoxycholic acid (GDCA), taurocholic acid (TCA), and taurochenodeoxycholic acid (TCDCA). Non-limiting examples of secondary bile acids are deoxycholic acid (DCA), lithocholic acid (LCA), ursodeoxycholic acid (UDCA), taurodeoxycholic acid (TDCA), taurolithocholic acid (TLCA), and tauroursodeoxycholic acid (TUDCA).

Non-limiting examples of secondary bile acids are deoxycholic acid (DCA), lithocholic acid (LCA), 3-oxolithocholic acid (3-oxoLCA), taurolithocholic acid (TLCA), taurodeoxycholic acid (TDCA), glycolithocholic (GLCA), glycodeoxycholic acid (GDCA), ursodeoxycholic acid (UDCA), tauroursodeoxycholic acid (TUDCA), glycoursodeoxycholic acid (GUDCA), and isoallolithocholic acid+isolithocholic acid (Isoallo-+IsoLCA).

Several diseases and disorders, such as inflammatory bowel disease, cancer (e.g., colorectal carcinoma), and pathogenic organism infections (e.g., Clostridium difficile infection), are associated with increased primary bile acids and reduced secondary bile acids. The primary bile acids are reduced, and the secondary bile acids are increased following fecal matter transplant (FMT). See, e.g., Seekatz, et al., Anaerobe (2018) 53:64-73. In some embodiments, administration of the bacterial strains or a composition as described herein reduces primary bile acids and/or increases secondary bile acids.

In some embodiments, the levels of primary bile acids are reduced by 2-fold to 100,000-fold following administration of the bacterial strains or any of the compositions described herein. In some embodiments, the levels of primary bile acids are reduced by 2-fold to 1,000-fold following administration of the bacterial strains or any of the compositions described herein. In some embodiments, the levels of primary bile acids are reduced by 10-fold to 1,000-fold following administration of the bacterial strains or any of the compositions described herein. In some embodiments, the levels of primary bile acids are reduced 20-fold to 10,000-fold following administration of the bacterial strains or any of the compositions described herein. In some embodiments, the levels of primary bile acids are reduced by 2-fold, 5-fold, 10-fold, 100-fold, 200-fold, 300-fold, 400-fold, 500-fold, 600-fold, 700-fold, 800-fold, 900-fold, 1,000-fold, 10,000-fold, 20,000-fold, 30,000-fold, 40,000-fold, 50,000-fold, 60,000-fold, 70,000-fold, 80,000-fold, 90,000-fold, or 100,000-fold following administration of the bacterial strains or any of the compositions described herein.

In some embodiments, the levels of secondary bile acids are increased by 2-fold to 10,000-fold following administration of the bacterial strains or any of the compositions described herein. In some embodiments, the levels of secondary bile acids are increased by 10-fold to 10,000-fold following administration of the bacterial strains or any of the compositions described herein. In some embodiments, the levels of secondary bile acids are increased by 10-fold to 1,000-fold following administration of the bacterial strains or any of the compositions described herein. In some embodiments, the levels of secondary bile acids are increased by 20-fold to 100-fold following administration of the bacterial strains or any of the compositions described herein. In some embodiments, the levels of secondary bile acids are increased by 10-fold, 20-fold, 50-fold, 100-fold, 200-fold, 300-fold, 400-fold, 500-fold, 600-fold, 700-fold, 800-fold, 900-fold, 1,000-fold, 2,000-fold, 3,000-fold, 4,000-fold, 5,000-fold, 6,000-fold, 7,000-fold, 8,000-fold, 9,000-fold, or 10,000-fold following administration of the bacterial strains or any of the compositions described herein.

Some embodiments of the compositions and methods described herein increase production of regulatory metabolites, such as short chain fatty acids (e.g., in the gastrointestinal tract of the subject), in a subject. In some embodiments, the methods involve administering to a subject one or more compositions containing bacterial strains that produce short chain fatty acids. SCFAs are abundant in healthy subjects and decreased in subjects having particular diseases and disorders.

SCFA produced in the gastrointestinal tract are thought to function as signaling molecules between the gut microbiota and the host organism, with the SCFA playing a role in local, intermediary and peripheral metabolism of the host. See, e.g., Morrison, et al. Gut Microbes (2016) 7 (3): 189-200.

Short chain fatty acids (SCFAs) are fatty acids containing six or fewer carbon atoms. They are produced when dietary fiber is fermented in the intestine. They are primarily absorbed in the portal vein following lipid digestion. SCFAs can affect the production of lipids, energy, and vitamins, as well as playing a critical role in maintaining intestinal epithelial cell membrane integrity. Examples of SCFA include, without limitation, formic acid, acetic acid, butyric acid, isobutyric acid, valeric acid, or isovaleric acid. In some embodiments, the SCFA is butyric acid (butyrate). In some embodiments, the SCFA is succinic acid. In some embodiments, the SCFA is valeric acid. In some embodiments, the SCFA is hexanoic acid. In some embodiments, the SCFA is propionic acid. In some embodiments, the SCFA is isovaleric acid. In some embodiments, the SCFA is isobutyric acid. In some embodiments, the SCFA is 2-methylbutyric acid. In some embodiments, the SCFA is acetic acid.

In some embodiments, SCFAs are increased by 2-fold to 10,000-fold following administration of the bacterial strains or any of the compositions described herein. In some embodiments, SCFAs are increased by 10-fold to 500-fold following administration of the bacterial strains or any of the compositions described herein. In some embodiments, SCFAs are increased by 2-fold to 250-fold following administration of the bacterial strains or any of the compositions described herein. In some embodiments, SCFAs are increased by 100-fold to 500-fold following administration of the bacterial strains or any of the compositions described herein. In some embodiments, SCFAs are increased by 2-fold, 5-fold, 10-fold, 20-fold, 30-fold, 40-fold, 50-fold, 60-fold, 70-fold, 80-fold, 90-fold, 100-fold, 200-fold, 300-fold, 400-fold, or 500-fold following administration of the bacterial strains or any of the compositions described herein.

Some aspects of the compositions and methods described herein increase production of regulatory metabolites, such as indoles (e.g., in the gastrointestinal tract of the subject). In some embodiments, the methods involve administering to a subject one or more of the compositions described herein containing bacterial strains that produce indoles. Indoles are abundant in healthy subjects and decreased in subjects having particular diseases and disorders.

Indoles are tryptophan derivatives and are produced when bacteria catabolize tryptophan in the intestine. Indoles produced in the gastrointestinal tract are thought to contribute to intestinal and systemic homeostasis and may hinder the development of hepatic pathologies such as alcoholic steatohepatitis and nonalcoholic fatty liver disease. See, e.g., Hendrikx et al., J Intern Med. (2019) 286(1):32-40. Examples of indoles include, without limitation, kynurenic acid, serotonin, nicotinic acid, indole, indole 3-acetic, and indole 3-propionic acid.

In some embodiments, the levels of indoles are increased by 2-fold to 10,000-fold following administration of the bacterial strains or any of the compositions described herein. In some embodiments, the levels of indoles are increased by 10-fold to 10,000-fold following administration of the bacterial strains or any of the compositions described herein. In some embodiments, the levels of indoles are increased by 10-fold to 1,000-fold following administration of the bacterial strains or any of the compositions described herein. In some embodiments, the levels of indoles are increased by 20-fold to 100-fold following administration of the bacterial strains or any of the compositions described herein. In some embodiments, the levels of indoles are increased by 10-fold, 20-fold, 50-fold, 100-fold, 200-fold, 300-fold, 400-fold, 500-fold, 600-fold, 700-fold, 800-fold, 900-fold, 1,000-fold, 2,000-fold, 3,000-fold, 4,000-fold, 5,000-fold, 6,000-fold, 7,000-fold, 8,000-fold, 9,000-fold, or 10,000-fold following administration of the bacterial strains or any of the compositions described herein.

In some embodiments, the compositions provided herein induce the proliferation and/or accumulation of regulatory T cells in the subject. As will be evident to one of ordinary skill in the art, regulatory T cells, also referred to as “Tregs,” are a subset of T lymphocytes that are generally thought to suppress an abnormal or excessive immune response and play a role in immune tolerance. Regulatory T cells may be identified based expression of the markers Foxp3 and CD4 (Foxp3+CD4+). The term regulatory T cells may also include Foxp3-negative regulatory T cells that are IL-10-producing CD4-positive T cells.

In some embodiments, the therapeutically effective amount is an amount sufficient to induce the proliferation and/or accumulation of Tregs in the subject (or in a sample obtained from a subject) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the amount of Tregs in a subject (e.g., a subject having or at risk of Clostridium difficile infection or recurrent infection) that has not received any of the compositions described herein or as compared to a fecal sample from the same subject that was collected prior to administration of any of the compositions.

As used herein, the phrase “induces proliferation and/or accumulation of regulatory T cells” refers to an effect of inducing the differentiation of immature T cells into regulatory T cells, which differentiation leads to the proliferation and/or the accumulation of regulatory T cells. Further, the meaning of “induces proliferation and/or accumulation of regulatory T cells” includes in vivo effects, in vitro effects, and ex vivo effects. In some embodiments, the proliferation and/or accumulation of regulatory T cells may be assessed by detecting and/or quantifying the number of cells that express markers of regulatory T cells (e.g., Foxp3 and CD4), for example by flow cytometry. In some embodiments, the proliferation and/or accumulation of regulatory T cells may be assessed by determining the activity of the regulatory T cells, such as the production of cytokines (e.g., IL-10).

In some embodiments, administration of the compositions described herein results in an increase the proliferation and/or accumulation of regulatory T cells (e.g., total Tregs or a specific subset of Treg) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the quantity of regulatory T cells in the subject (or particular site in the subject) prior to administration of the compositions. In some embodiments, administration of the compositions described herein results in an increase the proliferation and/or accumulation of regulatory T cells (e.g., total Tregs or a specific subset of Tregs) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the quantity of regulatory T cells in another subject (e.g., a reference subject) who did not receive the compositions.

In some embodiments, administration of the compositions described herein results in an increase in the proliferation and/or accumulation of regulatory T cells (e.g., total Tregs or a specific subset of Tregs) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the quantity of regulatory T cells in the subject (or particular site in the subject) prior to administration of the compositions. In some embodiments, administration of the compositions described herein results in an increase the proliferation and/or accumulation of regulatory T cells (e.g., total Tregs or a specific subset of Tregs) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the quantity of regulatory T cells in another subject (e.g., a reference subject) who did not receive the compositions.

In some embodiments, administration of the compositions described herein results in an increase in activity of regulatory T cells (e.g., total Tregs or a specific subset of Tregs) at a particular site (e.g., the gastrointestinal tract) in the subject. In some embodiments, administration of the compositions described herein results in an increase in activity of regulatory T cells (e.g., total Tregs or a specific subset of Tregs) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the activity of regulatory T cells in the subject (or particular site in the subject) prior to administration of the compositions. In some embodiments, administration of the compositions described herein results in an increase in activity of regulatory T cells (e.g., total Tregs or a specific subset of Tregs) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the activity of regulatory T cells in another subject (e.g., a reference subject) who did not receive the compositions.

In some embodiments, administration of the compositions described herein results in an increase in the activity of regulatory T cells (e.g., total Tregs or a specific subset of Tregs) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the activity of regulatory T cells in the subject (or particular site in the subject) prior to administration of the compositions. In some embodiments, administration of the compositions described herein results in an increase in the activity of regulatory T cells (e.g., total Tregs or a specific subset of Tregs) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the activity of regulatory T cells in another subject (e.g., a reference subject) who did not receive the compositions.

The abundance of regulatory T cells (e.g., total Tregs or a specific subset of Tregs) can be assessed by any suitable method, for example by detecting a cellular marker indicative of regulatory T cells (e.g., FoxP3), assessing a direct or indirect activity of regulatory T cells, and/or by measuring the production of one or more cytokines produced by regulatory T cells (e.g., IL-10).

Use with Antibiotics

Any of the methods described herein may further comprise administering an antibiotic to the subject prior to administration of the compositions described herein. In some embodiments, the subject was previously administered an antibiotic prior to administration of the compositions described herein. In some embodiments, administration of any of the compositions described herein is not preceded by administration of an antibiotic.

In some embodiments, the antibiotic is vancomycin, fidaxomycin or ridinilazole. Non-limiting examples of antibiotics that may be used in any of the methods provided herein include cephalosporin antibiotics cephalexin, cefuroxime, cefadroxil, cefazolin, cephalothin, cefaclor, cefamandole, cefoxitin, cefprozil, ceftobiprole, clindamycin, ceftriaxone, cefotaxime, cefazolin, cefoperazone, cefuroxime, cefmetazole, fluoroquinolone, ciprofloxacin, Levaquin, floxin, tequin, avelox, norflox, tetracycline, minocycline, oxytetracycline, doxycycline, amoxicillin, ampicillin, penicillin V, dicloxacillin, benzylpenicillin, carbenicillin, vancomycin, and methicillin, ertapenem, doripenem, imipenem/cilastatin, meropenem, clavulanate, tazobactam, piperacillin, ceftriaxone, cefotaxime, cefazolin, fluoroquinolone, imipenem, meropenem, metronidazole, fidaxomyxin, or ridinilazole. In some embodiments, any of the methods described herein may further comprise administering vancomycin to the subject prior to administration of the compositions described herein. In some embodiments, the method does not comprise administering vancomycin to the subject prior to administration of the compositions described herein. Vancomycin administration has been found to alter the composition of human gut microbiota. See, e.g., Reijnders et al. Cell Metabolism (2016) 24(1): 63-72. Without wishing to be bound by any particular theory, it is thought that administration of vancomycin may aid engraftment of the bacterial strain(s) of the compositions described herein, for example by removing other microbes present in the gastrointestinal tract.

In some embodiments, the vancomycin is administered at a dose (a single dose or multiple doses) in a sufficient amount to allow for colonization of one or more of the bacterial strains of the compositions described herein. In some embodiments, the vancomycin is administered to the subject once, as a single dose. In some embodiments, the vancomycin is administered to the subject in multiple doses. In some embodiments, the vancomycin is administered to the subject in at least 2, 3, 4, 5, 6, 7, 8, 9, 10, 11, 12, 13, 14, 15 or more doses. The multiple doses of vancomycin may be administered to the subject at regular intervals prior to administering any of the compositions described herein. In some embodiments, each of the multiple doses of vancomycin are administered on consecutive days (e.g., first dose on day 1, second dose of day 2, third dose on day 3, etc.). In some embodiments, the vancomycin is administered to the subject for 2, 3, 4, 5, 6, 7, 8, 9, 10, 11, 12, 13, 14, 15, or more consecutive days. In some embodiments, vancomycin is administered to the subject for one day. In some embodiments, vancomycin is administered to the subject each day for three consecutive days. In some embodiments, vancomycin is administered to the subject each day for five consecutive days. In some embodiments, the vancomycin is administered to the subject each day for seven consecutive days. In any of the embodiments described herein, a subject may be administered one or more doses of a first antibiotic followed by one or more doses of a second antibiotic.

In some embodiments, a single dose of any of the compositions described herein, or the first dose in a treatment regimen of multiple doses, is administered, the same day as the administration of the final dose of vancomycin. In some embodiments, a single dose of any of the compositions described herein, or the first dose in a treatment regimen of multiple doses, is administered, immediately following the administration of the final dose of vancomycin. In some embodiments, a single dose of any of the compositions described herein, or the first dose in a treatment regimen of multiple doses, is administered, the day after administration of the final dose of vancomycin. In some embodiments, a single dose of any of the compositions described herein, or the first dose in a treatment regimen of multiple doses, is administered, two days after administration of the final dose of vancomycin. In some embodiments, the methods provided herein allow for a wash out day between the final dose of vancomycin and the first dose of the composition. In some embodiments, a single dose of any of the compositions described herein, or the first dose in a treatment regimen of multiple doses, is administered, three days, four days, five days, six days, ten days or more, after administration of the final dose of vancomycin. In some embodiments, the methods provided herein allow for multiple wash out days between the final dose of vancomycin and the first dose of the composition. In some embodiments, the methods provided herein allow for two wash out days between the final dose of vancomycin and the first dose of the composition.

Each dose of the vancomycin may be the same amount of vancomycin or may be a different amount of vancomycin. In some embodiments, the vancomycin is administered in an amount sufficient to allow for colonization of one or more of the bacterial strains of the compositions described herein. In some embodiments, the subject is administered between about 50 mg and 1 g, 100 mg and 750 mg, 100 mg and 500 mg, 200 mg and 750 mg, 200 mg and 500 mg, 300 mg and 750 mg, 300 mg and 500 mg, 100 mg and 400 mg, 100 mg and 300 mg, 100 mg and 200 mg, 200 mg and 400 mg, 200 mg and 300 mg, or 450 mg to 550 mg vancomycin per day. As will be appreciated by one of skill in the art, the total amount of vancomycin administered to the subject per day may be administered in a single dose or between multiple doses, which in sum results in the total amount of vancomycin per day.

In some embodiments, the subject is administered about 500 mg vancomycin per day prior to administration of any of the compositions described herein. In some embodiments, 500 mg vancomycin per day is administered in a single dose (e.g., 500 mg). In some embodiments, 500 mg vancomycin per day is administered in multiple doses (e.g., 2, 3, 4, 5 or more), which in sum results in 500 mg vancomycin per day. In some embodiments, 500 mg vancomycin is administered in 4 doses of 125 mg vancomycin per day. In some embodiments, 500 mg vancomycin is administered to the subject for one day. In some embodiments, 500 mg vancomycin is administered to the subject per day for two days. In some embodiments, 500 mg vancomycin is administered to the subject per day for three days. In some embodiments, 500 mg vancomycin is administered to the subject per day for four days. In some embodiments, 500 mg vancomycin is administered to the subject per day for five days. In some embodiments, 500 mg vancomycin is administered to the subject per day for six days. In some embodiments, 500 mg vancomycin is administered to the subject per day for seven days. In some embodiments, 500 mg vancomycin is administered to the subject per day for eight days. In some embodiments, 500 mg vancomycin is administered to the subject per day for nine days. In some embodiments, 500 mg vancomycin is administered to the subject per day for ten days.

In some embodiments, 500 mg vancomycin is administered in 4 doses of 125 mg vancomycin per day for one day. In some embodiments, 500 mg vancomycin is administered in 4 doses of 125 mg vancomycin per day for two days. In some embodiments, 500 mg vancomycin is administered in 4 doses of 125 mg vancomycin per day for three days. In some embodiments, 500 mg vancomycin is administered in 4 doses of 125 mg vancomycin per day for four days. In some embodiments, 500 mg vancomycin is administered in 4 doses of 125 mg vancomycin per day for five days. In some embodiments, 500 mg vancomycin is administered in 4 doses of 125 mg vancomycin per day for six days. In some embodiments, 500 mg vancomycin is administered in 4 doses of 125 mg vancomycin per day for seven days. In some embodiments, 500 mg vancomycin is administered in 4 doses of 125 mg vancomycin per day for eight days. In some embodiments, 500 mg vancomycin is administered in 4 doses of 125 mg vancomycin per day for nine days. In some embodiments, 500 mg vancomycin is administered in 4 doses of 125 mg vancomycin per day for ten days.

In some embodiments, the vancomycin is administered according to a pulse tapered-regime. See e.g., Sirbu et al., Clinical Infectious Diseases (2017) 65: 1396-1399.

In some embodiments, the vancomycin is administered to the subject at least 1, 2, 3, 4, 5, 6, 7 days or more prior to administration of the compositions described herein. In some embodiments, administration of vancomycin is terminated at least one day (e.g., 1, 2, 3, 4, 5, or more) prior to administration of any of the compositions described herein. In some embodiments, administration of vancomycin is terminated two days prior to administration of any of the compositions described herein.

In some embodiments, additional antibiotics are administered in combination with the vancomycin regimes provided herein.

It should be appreciated, in some embodiments, that any of the vancomycin doses or administration regimens may be combined with any of the composition doses or administration regimens provided herein.

In some embodiments, the disclosure provides methods comprising administering one or more antibiotics to the subject and subsequently administering any of the compositions to the subject once, twice, 3 times, 4 times, 5 times, 6 times, 7 times, 8 times, 9 times, 10 times, 11 times, 12 times, 13 times, at least 14 times or more. In some embodiments, the disclosure provides methods comprising administering one or more antibiotics to the subject and subsequently administering any of the compositions described herein to the subject in multiple doses at a regular interval, such as every 2 weeks, every month, every 2 months, every 3 months, every 4 months, every 5 months, every 6 months, or more. In some embodiments, one dose of any of the compositions described herein is administered and a second dose of the composition is administered the following day (e.g., consecutive day). In some embodiments, one dose of any of the compositions described herein is administered and each of the additional doses of the composition are administered on consecutive days (e.g., first dose on day 1, second dose on day 2, third dose on day 3, etc.).

In some embodiments, multiple doses are administered over multiple days. In some embodiments, the subject is administered at least 1.0×10⁹, 1.1×10⁹, 1.2×10⁹, 1.3×10⁹, 1.4×10⁹, 1.5×10⁹, 1.6×10⁹, 1.7×10⁹, 1.8×10⁹, 1.9×10⁹, 2.0×10⁹, 2.1×10⁹, 2.2×10⁹, 2.3×10⁹, 2.4×10⁹, 2.5×10⁹, 2.6×10⁹, 2.7×10⁹, 2.8×10⁹, 2.9×10⁹, 3.0×10⁹, 3.1×10⁹, 3.2×10⁹, 3.3×10⁹, 3.4×10⁹, 3.5×10⁹, 3.6×10⁹, 3.7×10⁹, 3.8×10⁹, 3.9×10⁹, 4.0×10⁹, 4.1×10⁹, 4.2×10⁹, 4.3×10⁹, 4.4×10⁹, 4.5×10⁹, 4.6×10⁹, 4.7×10⁹, 4.8×10⁹, 4.9×10⁹, 5.0×10⁹, 5.1×10⁹, 5.2×10⁹, 5.3×10⁹, 5.4×10⁹, 5.5×10⁹, 5.6×10⁹, 5.7×10⁹, 5.8×10⁹, 5.9×10⁹, 6.0×10⁹, 6.1×10⁹, 6.2×10⁹, 6.3×10⁹, 6.4×10⁹, 6.5×10⁹, 6.6×10⁹, 6.7×10⁹, 6.8×10⁹, 6.9×10⁹, 7.0×10⁹, 7.1×10⁹, 7.2×10⁹, 7.3×10⁹, 7.4×10⁹, 7.5×10⁹, 7.6×10⁹, 7.7×10⁹, 7.8×10⁹, 7.9×10⁹, 8.0×10⁹, 8.1×10⁹, 8.2×10⁹, 8.3×10⁹, 8.4×10⁹, 8.5×10⁹, 8.6×10⁹, 8.7×10⁹, 8.8×10⁹, 8.9×10⁹, 9.0×10⁹, 9.1×10⁹, 9.2×10⁹, 9.3×10⁹, 9.4×10⁹, 9.5×10⁹, 9.6×10⁹, 9.7×10⁹, 9.8×10⁹, or 9.9×10⁹ colony forming units of bacteria per day. In some embodiments, the subject is administered at least 1.0×10¹⁰, 1.1×10¹⁰, 1.2×10¹⁰, 1.3×10¹⁰, 1.4×10¹⁰, 1.5×10¹⁰, 1.6×10¹⁰, 1.7×10¹⁰, 1.8×10¹⁰, 1.9×10¹⁰, 2.0×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3.0×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4.0×10¹⁰, 4.1×10¹⁰, 4.2×10¹⁰, 4.3×10¹⁰, 4.4×10¹⁰, 4.5×10¹⁰, 4.6×10¹⁰, 4.7×10¹⁰, 4.8×10¹⁰, 4.9×10¹⁰, 5.0×10¹⁰, 5.1×10¹⁰, 5.2×10¹⁰, 5.3×10¹⁰, 5.4×10¹⁰, 5.5×10¹⁰, 5.6×10¹⁰, 5.7×10¹⁰, 5.8×10¹⁰, 5.9×10¹⁰, 6.0×10¹⁰, 6.1×10¹⁰, 6.2×10¹⁰, 6.3×10¹⁰, 6.4×10¹⁰, 6.5×10¹⁰, 6.6×10¹⁰, 6.7×10¹⁰, 6.8×10¹⁰, 6.9×10¹⁰, 7.0×10¹⁰, 7.1×10¹⁰, 7.2×10¹⁰, 7.3×10¹⁰, 7.4×10¹⁰, 7.5×10¹⁰, 7.6×10¹⁰, 7.7×10¹⁰, 7.8×10¹⁰, 7.9×10¹⁰, 8.0×10¹⁰, 8.1×10¹⁰, 8.2×10¹⁰, 8.3×10¹⁰, 8.4×10¹⁰, 8.5×10¹⁰, 8.6×10¹⁰, 8.7×10¹⁰, 8.8×10¹⁰, 8.9×10¹⁰, 9.0×10¹⁰, 9.1×10¹⁰, 9.2×10¹⁰, 9.3×10¹⁰, 9.4×10¹⁰, 9.5×10¹⁰, 9.6×10¹⁰, 9.7×10¹⁰, 9.8×10¹⁰, or 9.9×10¹⁰ colony forming units of bacteria in total. In some embodiments, the subject is administered at least 1.0×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2.0×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3.0×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4.0×10¹¹, 4.1×10¹¹, 4.2×10¹¹, 4.3×10¹¹, 4.4×10¹¹, 4.5×10¹¹, 4.6×10¹¹, 4.7×10¹¹, 4.8×10¹¹, 4.9×10¹¹, 5.0×10¹¹, 5.1×10¹¹, 5.2×10¹¹, 5.3×10¹¹, 5.4×10¹¹, 5.5×10¹¹, 5.6×10¹¹, 5.7×10¹¹, 5.8×10¹¹, 5.9×10¹¹, 6.0×10¹¹, 6.1×10¹¹, 6.2×10¹¹, 6.3×10¹¹, 6.4×10¹¹, 6.5×10¹¹, 6.6×10¹¹, 6.7×10¹¹, 6.8×10¹¹, 6.9×10¹¹, 7.0×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8.0×10¹¹, 8.1×10¹¹, 8.2×10¹¹, 8.3×10¹¹, 8.4×10¹¹, 8.5×10¹¹, 8.6×10¹¹, 8.7×10¹¹, 8.8×10¹¹, 8.9×10¹¹, 9.0×10¹¹, 9.1×10¹¹, 9.2×10¹¹, 9.3×10¹¹, 9.4×10¹¹, 9.5×10¹¹, 9.6×10¹¹, 9.7×10¹¹, 9.8×10¹¹, or 9.9×10¹⁰ colony forming units of bacteria in total.

In some embodiments, the subject is treated by multiple cycles, each of which may involve a period of administering multiple doses of any of the compositions described herein, followed by a period during which the compositions are not administered. In some embodiments, each cycle further involves administering antibiotics prior to administering any of the compositions described herein. In some embodiments, the subject is treated by multiple cycles, each of which may involve a period of administering multiple doses of any of the compositions described herein for at least 7 days, followed by a period during which the compositions are not administered. In some embodiments, the subject is treated by multiple cycles, each of which may involve a period of administering multiple doses of any of the compositions described herein for at least 14 days, followed by a period during which the compositions are not administered. In some embodiments, a cycle further comprises determining whether the subject is colonized by one or more of the bacterial strains of the composition, and administering the composition if the one or more of the bacteria of the composition are not detected. In some embodiments, the cycle is repeated after a period of time, such as after 2, 3, 4, 5, 6, 7, 8, 9, 10, 11, or 12 months.

In one aspect, the disclosure provides methods comprising administering one or more antibiotics to the subject and subsequently administering any of the bacterial compositions as multiple daily doses of the compositions. In some embodiments, the compositions are administered on a daily basis for 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 month, 2 months, 3 months, 4 months, 5 months, 6 months, 7 months, 8 months, 9 months, 10 months, 11 months, 12 months, or more.

In some embodiments, the compositions are administered on a daily basis for a period of time, followed by a period of time during which the compositions are not administered. In some embodiments, the methods may involve administering the compositions to the subject on a daily basis for a second period of time (e.g., a cycle), which may be followed by a second period of time during which the compositions are not administered.

In one aspect the disclosure provides methods comprising the administration of an antibiotic (e.g., vancomycin) followed by the administration of a composition provided herein, wherein the administration of an antibiotic (e.g., vancomycin) is followed by the administration of a single dose or multiple doses of the composition. In some embodiments, administration of an antibiotic (e.g., vancomycin) followed by the administration of a single dose or multiple doses of the composition results in an increase in the abundance of bacterial strains of the compositions in the microbiome of the subject (engraftment) compared to the administration of a composition without the administration of the antibiotic. In some embodiments, administration of an antibiotic (e.g., vancomycin) followed by the administration of a single dose or multiple doses of the composition results in an increase in the duration of the colonization of bacterial strains of the composition in the microbiome of the subject (e.g., up to 6 months) compared to the administration of a composition without the administration of the antibiotic.

In some embodiments, administration of an antibiotic (e.g., vancomycin) followed by the administration of a single dose or multiple doses of the composition results in an increase in the rate of engraftment of the initial amount of the bacterial strains of the composition in the microbiome of the subject by between ten- to one hundred-fold (e.g., within the first 48 hours) compared to the administration of a composition without the administration of the antibiotic.

In some embodiments, administration of an antibiotic (e.g., vancomycin) followed by the administration of a single dose or multiple doses of the composition results in a greater number (amount) of subjects having all of the bacterial strains of the composition present in their microbiome as compared to compared to the administration of a composition without the administration of the antibiotic.

In some embodiments, administration of an antibiotic (e.g., vancomycin) followed by the administration of multiple doses of the composition results in a higher abundance of the bacterial strains of the composition in the microbiome of the subject as compared to the administration of a single dose of the composition. In some embodiments, the disclosure provides methods comprising the administration of a composition provided herein, wherein the administration of multiple doses of the composition results in a higher abundance of the bacterial strains of the composition in the microbiome of the subject compared to the administration of a single dose of the composition.

In some embodiments, administration of an antibiotic (e.g., vancomycin) followed by the administration of multiple doses of the composition results in a greater number (amount) of subjects having all of the bacterial strains of the composition present in their microbiome as compared to the administration of a single dose of the composition. In some embodiments, the disclosure provides methods comprising the administration of a composition provided herein, wherein the administration of multiple doses of the results in a greater number (amount) of subject having all of the bacterial strains of the composition in their microbiome as compared to the administration of a single dose of the composition.

### Dosage Regimes

A physician, veterinarian or other trained practitioner, can start doses of the composition at levels lower than that required to achieve the desired therapeutic effect and gradually increase the dosage until the desired effect (e.g., colonization of a microbiome, engraftment of one or more bacterial strains of the composition, treatment of pathogenic (e.g., C. difficile) infection) is achieved. In general, effective doses of the compositions described herein for the prophylactic treatment of groups of people as described herein vary depending upon many different factors, including routes of administration, physiological state of the subject, whether the subject is a human or an animal, other medications administered, and the therapeutic effect desired. Dosages need to be titrated to optimize safety and efficacy. In some embodiments, the dosing regimen entails oral administration of a dose of any of the compositions described herein. In some embodiments, the dosing regimen entails oral administration of a single dose of any of the compositions described herein. In some embodiments, the dosing regimen entails oral administration of multiple doses of any of the compositions described herein. In some embodiments, any of the compositions described herein are administered the subject once, twice, 3 times, 4 times, 5 times, 6 times, 7 times, 8 times, 9 times, at least 10 times, at least 11 times, at least 12 times, at least 13 times, at least 14 times, or more. In some embodiments, any of the compositions described herein are administered the subject in multiple doses at a regular interval, such as every day, every 2 days, every 3 days, every 4 days, every 5 days, every 6 days, every week, every 2 weeks, every month, every 2 months, every 3 months, every 4 months, every 5 months, every 6 months, or more. In some embodiments, one dose of any of the compositions described herein is administered and a second dose of the composition is administered the following day (e.g., consecutive day). In some embodiments, one dose of any of the compositions described herein is administered and each of the additional doses of the composition are administered on consecutive days (e.g., first dose on day 1, second dose of day 2, third dose on day 3, etc.).

In some embodiments, the disclosure relates to methods comprising administration of multiple doses of the compositions. In some embodiments, the disclosure relates to methods comprising administration of antibiotic (e.g., vancomycin) followed by multiple doses of the compositions. In some embodiments, administration of multiple doses of the compositions described herein provides enhanced colonization (engraftment) of one or more bacterial strains of the compositions as compared to administration of a single dose of the composition. In some embodiments, administration of multiple doses of the compositions described herein provides enhanced recovery of one or more bacterial strains of the compositions as compared to administration of a single dose of the composition. In some embodiments, administration of multiple doses of the compositions described herein provides increased abundance of one or more bacterial strains of the compositions as compared to administration of a single dose of the composition. In some embodiments, administration of multiple doses of the compositions described herein provides an increase in the number of subjects that were colonized with of all of bacterial strains of the compositions as compared to administration of a single dose of the composition. In some embodiments, administration of multiple doses of the compositions described herein provides enhanced durability of colonization with one or more bacterial strains of the compositions as compared to administration of a single dose of the composition. In some embodiments, administration of multiple doses of the compositions described herein provides durable colonization of some or all of the bacterial strains of the compositions described herein over an extended period of time as compared to administration of a single dose of the composition. In some embodiments, administration of multiple doses of the compositions described herein provides durable colonization (e.g., up to 6 months) of one or more bacterial strains of the compositions as compared to administration of a single dose of the composition. In some embodiments, administration of multiple doses of the compositions described herein provides durable colonization (e.g., up to 6 months or longer) of some or all of the bacterial strains of the compositions as compared to administration of a single dose of the composition. It should further be appreciated that administration of multiple doses may result in a combination of the results described. Thus, for example, in some embodiments, administration of multiple doses of the compositions described herein provides enhanced colonization (engraftment) and increased rate of recovery of one or more bacterial strains of the compositions as compared to administration of a single dose of the composition.

In some embodiments, administration of multiple doses of the compositions described herein provides enhanced colonization (engraftment) of one or more bacterial strains of the compositions as compared to administration of a single dose of the composition. In some embodiments, administration of a single dose of the composition results in the same or a similar level of engraftment (e.g., total bacteria) as administration of multiple doses of the composition, however the engraftment may be dominated by one bacterial strain or only a subset of the bacterial strains of the compositions.

### Dosage Amounts

Dosages of the active ingredients in the compositions described herein can be varied so as to obtain an amount of the active ingredient which is effective to achieve the desired response for a particular subject, composition, and mode of administration, without being toxic or having an adverse effect on the subject. The selected dosage level depends upon a variety of factors including the activity of the particular compositions employed, the route of administration, the time of administration, the duration of the treatment, other drugs, compounds and/or materials used in combination with the particular compositions employed, the age, sex, weight, condition, general health, and prior medical history of the subject being treated, and like factors.

The compositions, including the pharmaceutical compositions disclosed herein, include compositions that contain selected bacterial strains. The amount of bacteria, including the amount of bacteria of each of the bacterial strains, in the compositions, including pharmaceutical compositions, may be expressed in weight, number of bacteria and/or CFUs (colony forming units). In some embodiments, the compositions, including pharmaceutical compositions, comprise about 10, about 10², about 10³, about 10⁴, about 10⁵, about 10⁶, about 10⁷, about 10⁸, about 10⁹, about 10¹⁰, about 10¹¹, about 10¹², about 10¹³ or more of each of the bacterial strains per dosage amount. In some embodiments, the compositions, including pharmaceutical compositions, comprise about 10, about 10², about 10³, about 10⁴, about 10⁵, about 10⁶, about 10⁷, about 10⁸, about 10⁹, about 10¹⁰, about 10¹¹, about 10¹², about 10¹³ or more total bacteria per dosage amount. It should further be appreciated that bacteria of each of the bacterial strains may be present in different amounts. Thus, for instance, as a non-limiting example, the composition may include 10³ of bacteria A, 10⁴ of bacteria B and 10⁶ of bacteria C. In some embodiments, the compositions, including pharmaceutical composition, comprise about 10, about 10², about 10³, about 10⁴, about 10⁵, about 10⁶, about 10⁷, about 10⁸, about 10⁹, about 10¹⁰, about 10¹¹, about 10¹², about 10¹³ or more CFUs of each of the bacterial strains per dosage amount. In some embodiments, the compositions, including pharmaceutical compositions, comprise about 10¹, about 10², about 10³, about 10⁴, about 10⁵, about 10⁶, about 10⁷, about 10⁸, about 10⁹, about 10¹⁰, about 10¹¹, about 10¹², about 10¹³ or more CFUs in total for all of the bacterial strains combined per dosage amount. As discussed above, bacteria of each of the bacterial strains may be present in different amounts. In some embodiments, the compositions, including pharmaceutical compositions, contain about 10⁻⁷, about 10⁻⁶, about 10⁻⁵, about 10⁻⁴, about 10⁻³, about 10⁻², about 10⁻¹ or more grams of bacteria of each of the bacterial strains in the composition per dosage amount. In some embodiments, the compositions, including pharmaceutical compositions, contain about 10⁻⁷, about 10⁻⁶, about 10⁻⁵, about 10⁻⁴, about 10⁻³, about 10⁻², about 10⁻¹ or more grams of bacteria in total for all of the bacterial strains combined per dosage amount. In some embodiments, the compositions, including pharmaceutical compositions, comprise about 10⁹ CFUs in total for all of the bacterial strains combined per dosage amount. In some embodiments, the compositions, including pharmaceutical compositions, comprise about 10¹⁰ CFUs in total for all of the bacterial strains combined per dosage amount.

In some embodiments, the compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total bacteria or colony forming units. In some embodiments, the compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total bacteria or colony forming units per milliliter.

In some embodiments, the compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² bacterial cells or colony forming units of bacteria. In some embodiments, the compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² bacterial cells or colony forming units of bacteria per milliliter.

In some embodiments, the composition includes between 10¹⁰ and 10¹², inclusive, bacterial cells or colony forming units of bacteria per milliliter.

In some embodiments, if a composition includes more than one bacterial strain, each bacterial strain is present in the composition in the same quantities, in terms of bacterial cells or CFUs. For example, a composition comprising Clostridium symbiosum and Blautia producta may comprise about 1×10⁸ CFU of Clostridium symbiosum and 1×10⁸ CFU of Blautia producta per milliliter, or may comprise 1×10⁸ Clostridium symbiosum cells and 1×10⁸ Blautia producta cells per milliliter.

In some embodiments, the compositions disclosed herein containing more than one bacterial strain contain about 10, about 10², about 10³, about 10⁴, about 10⁵, about 10⁶, about 10⁷, about 10⁸, about 10⁹, about 10¹⁰, about 10¹¹, about 10¹², about 10¹³ or more of each bacterial strain. In some embodiments, the compositions disclosed herein containing more than one bacterial strain contain about 10, about 10², about 10³, about 10⁴, about 10⁵, about 10⁶, about 10⁷, about 10⁸, about 10⁹, about 10¹⁰, about 10¹¹, about 10¹², about 10¹³ or more of each bacterial strain per milliliter. In some embodiments, the compositions disclosed herein containing more than one bacterial strain contain about 10, about 10², about 10³, about 10⁴, about 10⁵, about 10⁶, about 10⁷, about 10⁸, about 10⁹, about 10¹⁰, about 10¹¹, about 10¹², about 10¹³ or more colony forming units (CFUs) of each bacterial strain. In some embodiments, the compositions disclosed herein containing more than one bacterial strain contain about 10, about 10², about 10³, about 10⁴, about 10⁵, about 10⁶, about 10⁷, about 10⁸, about 10⁹, about 10¹⁰, about 10¹¹, about 10¹², about 10¹³ or more colony forming units (CFUs) of each of the bacterial strains per milliliter.

In some embodiments, the compositions disclosed herein containing more than one bacterial strain contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰ between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total bacterial cells or colony forming units of each bacterial strain. In some embodiments, the compositions disclosed herein containing more than one bacterial strain contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² bacterial cells or colony forming units of each bacterial strain per milliliter.

In some embodiments, the compositions disclosed herein containing more than one bacterial strain contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³ between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² bacterial cells or colony forming units of each bacterial strain. In some embodiments, the compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² bacterial cells or colony forming units of each bacterial strain per milliliter.

As described herein, any of the compositions described herein may be administered to a subject in one dose or in multiple doses (e.g., initial administration), which may be followed by one or more additional doses of any of the compositions described herein. In some embodiments, any of composition described herein may be administered to a subject in one dose or in multiple doses in an initial administration, followed by one or more additional doses of a composition comprising the same one or more bacterial strains as the composition of the initial administration. In some embodiments, any of composition described herein may be administered to a subject in one dose or in multiple doses in an initial administration, followed by one or more additional doses of a composition comprising more total bacteria (colony-forming units) relative to the initial administration of the composition. In some embodiments, any of composition described herein may be administered to a subject in one dose or in multiple doses in an initial administration, followed by one or more additional doses of a composition comprising fewer total bacteria (colony-forming units) relative to the initial administration of the composition. In some embodiments, the initial administration includes at least 1, 2, 3, 4, 5, 6, 7, 8, 9, 10, 11, 12, 13, 14, 15 or more doses of any of the compositions described herein. In some embodiments, the additional administration includes at least 1, 2, 3, 4, 5, 6, 7, 8, 9, 10, 11, 12, 13, 14, 15 or more doses of any of the compositions described herein. In some embodiments, the initial administration comprises two doses of any of the composition and the additional administration comprises three doses of any of the compositions described herein.

In some embodiments, the subject has not received a dose of an antibiotic prior to administration of the bacterial composition. In some embodiments, the subject has not been administered an antibiotic at least 1, at least 2, at least 3, at least 5, at least 10, at least 15, at least 20, at least 25, at least 30, at least 60, at least 90, at least 120, at least 180, or at least 360 days prior to administration of the compositions provided herein. In some embodiments, the subject is treated with an amount of antibiotics sufficient to allow for the grafting of the one or more strains of the bacterial compositions provided herein.

In some embodiments, the dosing regimen entails administration of multiple doses of any of the compositions described herein. In some embodiments, the composition is administered orally to the subject once, twice, 3 times, 4 times, 5 times, 6 times, 7 times, 8 times, 9 times, at least 10 times, at least 11 times, at least 12 times, at least 13 times, at least 14 times, or more. In some embodiments, any of the compositions described herein are administered to the subject in multiple doses at a regular interval, such as every day, every 2 days, every 3 days, every 4 days, every 5 days, every 6 days, every week, every 2 weeks, every 4 weeks, every month, every 2 months, every 3 months, every 4 months, every 5 months, every 6 months, or more. In some embodiments, one dose of any of the compositions described herein is administered and a second dose of the composition is administered the following day (e.g., consecutive days). In some embodiments, one dose of any of the compositions described herein is administered and each of the additional doses of the composition are administered on consecutive days (e.g., first dose on day 1, second dose of day 2, third dose on day 3, etc.).

In some embodiments, the subject is administered a single dose of an antibiotic prior to the administration of any of the bacterial compositions described herein. In some embodiments, the subject is administered multiple doses of an antibiotic prior to the administration of any of the bacterial compositions described herein. In some embodiments, the subject is administered at least 2, 3, 4, 5 or more doses of an antibiotic prior to the administration of any of the bacterial compositions described herein. In some embodiments, the subject is administered a dose of an antibiotic at substantially the same time as the administration of any of the bacterial compositions described herein. Examples of antibiotics that can be administered include, without limitation, vancomycin, kanamycin, gentamicin, colistin, metronidazole, clindamycin, fidaxomicin, penicillin, streptomycin, and cefoperazone.

In some embodiments in any one the methods provided herein, a subject is evaluated for the presence of one or more of the bacterial strains of the compositions described herein in the microbiome. In some embodiments, if the subject does not have, or only has a low level of one or more of the bacterial strains of the bacterial compositions described herein in the microbiome, any one of the compositions provided herein, or one or more further doses of any one of the compositions provided herein, may be administered.

In some embodiments, the subject is evaluated for the presence of and/or abundance of one or more bacterial strains of the bacterial compositions described herein in the microbiome. In some embodiments, if one or more bacterial strains of the compositions are detected at a level above a threshold level, no further compositions or food products containing the bacterial strains are administered to the subject. In some embodiments, if one or more bacterial strains of the compositions colonize the subject to a level above a threshold level, no further compositions or food products containing the bacterial strains are administered to the subject.

### Food Products

Some aspects of the disclosure also provide food products comprising any of the compositions provided herein and a nutrient. Also within the scope of the disclosure are food products comprising any of the bacterial strains described herein and a nutrient. Food products are, in general, intended for the consumption of a human or an animal. Any of the compositions described herein may be present in a food product. In some embodiments, the bacterial strains are present in spore form in a food product. In some embodiments, the bacterial strains are present in vegetative form in a food product. In some embodiments, the food product comprises both vegetative bacteria and bacteria in spore form. The compositions disclosed herein can be used in a food or beverage, such as a health food or beverage, a food or beverage for infants, a food or beverage for pregnant women, athletes, senior citizens or other specified group, a functional food, a beverage, a food or beverage for specified health use, a dietary supplement, a food or beverage for patients, or an animal feed.

Non-limiting examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, soups; dairy products such as milk, dairy beverages, ice creams, cheeses, and yogurts; fermented products such as fermented soybean pastes, yogurts, fermented beverages, and pickles; bean products; various confectionery products such as Western confectionery products including biscuits, cookies, and the like, Japanese confectionery products including steamed bean-jam buns, soft adzuki-bean jellies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies.

Food products containing the bacterial strains described herein may be produced using any suitable methods and may contain the same amount of bacteria (e.g., by weight, amount, or CFU) as the compositions (e.g., pharmaceutical compositions) provided herein. Selection of an appropriate amount of bacteria in the food product may depend on various factors, including for example, the serving size of the food product, the frequency of consumption of the food product, the specific bacterial strains contained in the food product, the amount of water in the food product, and/or additional conditions for survival of the bacteria in the food product.

Examples of food products which may be comprise any of the bacterial strains described herein include, without limitation, a beverage, a drink, a bar, a snack, a dairy product, a confectionery product, a cereal product, a ready-to-eat product, a nutritional formula, such as a nutritional supplement, or a food or beverage additive.

Strain 1 (VE303-1)
Clostridium bolteae 16S ribosomal RNA coding sequence (16S rDNA)
(SEQ ID NO: 1)
ATGAGAGTTTGATCCTGGCTCAGGATGAACGCTGGCGGCGTGCCTAACACATGCAAGTCGAACGAAGCAATTAAA
ATGAAGTTTTCGGATGGATTTTTGATTGACTGAGTGGCGGACGGGTGAGTAACGCGTGGATAACCTGCCTCACAC
TGGGGGATAACAGTTAGAAATGACTGCTAATACCGCATAAGCGCACAGTACCGCATGGTACGGTGTGAAAAACTC
CGGTGGTGTGAGATGGATCCGCGTCTGATTAGCCAGTTGGCGGGGTAACGGCCCACCAAAGCGACGATCAGTAGC
CGACCTGAGAGGGTGACCGGCCACATTGGGACTGAGACACGGCCCAAACTCCTACGGGAGGCAGCAGTGGGGAAT
ATTGCACAATGGGCGAAAGCCTGATGCAGCGACGCCGCGTGAGTGAAGAAGTATTTCGGTATGTAAAGCTCTATC
AGCAGGGAAGAAAATGACGGTACCTGACTAAGAAGCCCCGGCTAACTACGTGCCAGCAGCCGCGGTAATACGTAG
GGGGCAAGCGTTATCCGGATTTACTGGGTGTAAAGGGAGCGTAGACGGCGAAGCAAGTCTGAAGTGAAAACCCAG
GGCTCAACCCTGGGACTGCTTTGGAAACTGTTTTGCTAGAGTGTCGGAGAGGTAAGTGGAATTCCTAGTGTAGCG
GTGAAATGCGTAGATATTAGGAGGAACACCAGTGGCGAAGGCGGCTTACTGGACGATAACTGACGTTGAGGCTCG
AAAGCGTGGGGAGCAAACAGGATTAGATACCCTGGTAGTCCACGCCGTAAACGATGAATGCTAGGTGTTGGGGGG
CAAAGCCCTTCGGTGCCGTCGCAAACGCAGTAAGCATTCCACCTGGGGAGTACGTTCGCAAGAATGAAACTCAAA
GGAATTGACGGGGACCCGCACAAGCGGTGGAGCATGTGGTTTAATTCGAAGCAACGCGAAGAACCTTACCAAGTC
TTGACATCCTCTTGACCGGCGTGTAACGGCGCCTTCCCTTCGGGGCAGGAGAGACAGGTGGTGCATGGTTGTCGT
CAGCTCGTGTCGTGAGATGTTGGGTTAAGTCCCGCAACGAGCGCAACCCTTATCCTTAGTAGCCAGCAGGTAGAG
CTGGGCACTCTAGGGAGACTGCCAGGGATAACCTGGAGGAAGGTGGGGATGACGTCAAATCATCATGCCCCTTAT
GATTTGGGCTACACACGTGCTACAATGGCGTAAACAAAGGGAAGCAAGACAGTGATGTGGAGCAAATCCCAAAAA
TAACGTCCCAGTTCGGACTGTAGTCTGCAACCCGACTACACGAAGCTGGAATCGCTAGTAATCGCGAATCAGAAT
GTCGCGGTGAATACGTTCCCGGGTCTTGTACACACCGCCCGTCACACCATGGGAGTCAGCAACGCCCGAAGTCAG
TGACCCAACTCGCAAGAGAGGGAGCTGCCGAAGGCGGGGCAGGTAACTGGGGTGAAGTCGTAACAAGGTAGCCGT
ATCGGAAGGTGCGGCTGGATCACCTCCTTT
Strain 2 (VE303-2)
Anaerotruncus colihominis 16S ribosomal RNA coding sequence (16S
rDNA)
(SEQ ID NO: 2)
CAAAGAGTTTGATCCTGGCTCAGGACGAACGCTGGCGGCGCGCCTAACACATGCAAGTCGAACGGAGCTTACGTT
TTGAAGTTTTCGGATGGATGAATGTAAGCTTAGTGGCGGACGGGTGAGTAACACGTGAGCAACCTGCCTTTCAGA
GGGGGATAACAGCCGGAAACGGCTGCTAATACCGCATGATGTTGCGGGGGCACATGCCCCTGCAACCAAAGGAGC
AATCCGCTGAAAGATGGGCTCGCGTCCGATTAGCCAGTTGGCGGGGTAACGGCCCACCAAAGCGACGATCGGTAG
CCGGACTGAGAGGTTGAACGGCCACATTGGGACTGAGACACGGCCCAGACTCCTACGGGAGGCAGCAGTGGGGGA
TATTGCACAATGGGCGAAAGCCTGATGCAGCGACGCCGCGTGAGGGAAGACGGTCTTCGGATTGTAAACCTCTGT
CTTTGGGGAAGAAAATGACGGTACCCAAAGAGGAAGCTCCGGCTAACTACGTGCCAGCAGCCGCGGTAATACGTA
GGGAGCAAGCGTTGTCCGGAATTACTGGGTGTAAAGGGAGCGTAGGCGGGATGGCAAGTAGAATGTTAAATCCAT
CGGCTCAACCGGTGGCTGCGTTCTAAACTGCCGTTCTTGAGTGAAGTAGAGGCAGGCGGAATTCCTAGTGTAGCG
GTGAAATGCGTAGATATTAGGAGGAACACCAGTGGCGAAGGCGGCCTGCTGGGCTTTAACTGACGCTGAGGCTCG
AAAGCGTGGGGAGCAAACAGGATTAGATACCCTGGTAGTCCACGCCGTAAACGATGATTACTAGGTGTGGGGGGA
CTGACCCCTTCCGTGCCGCAGTTAACACAATAAGTAATCCACCTGGGGAGTACGGCCGCAAGGTTGAAACTCAAA
GGAATTGACGGGGGCCCGCACAAGCAGTGGAGTATGTGGTTTAATTCGAAGCAACGCGAAGAACCTTACCAGGTC
TTGACATCGGATGCATAGCCTAGAGATAGGTGAAGCCCTTCGGGGCATCCAGACAGGTGGTGCATGGTTGTCGTC
AGCTCGTGTCGTGAGATGTTGGGTTAAGTCCCGCAACGAGCGCAACCCTTATTATTAGTTGCTACGCAAGAGCAC
TCTAATGAGACTGCCGTTGACAAAACGGAGGAAGGTGGGGATGACGTCAAATCATCATGCCCCTTATGACCTGGG
CTACACACGTACTACAATGGCACTAAAACAGAGGGCGGCGACACCGCGAGGTGAAGCGAATCCCGAAAAAGTGTC
TCAGTTCAGATTGCAGGCTGCAACCCGCCTGCATGAAGTCGGAATTGCTAGTAATCGCGGATCAGCATGCCGCGG
TGAATACGTTCCCGGGCCTTGTACACACCGCCCGTCACACCATGGGAGTCGGTAACACCCGAAGCCAGTAGCCTA
ACCGCAAGGGGGGCGCTGTCGAAGGTGGGATTGATGACTGGGGTGAAGTCGTAACAAGGTAGCCGTATCGGAAGG
TGCGGCTGGATCACCTCCTTT
Strain 3 (VE303-3)
Sellimonas intestinalis 16S ribosomal RNA coding sequence (16S
rDNA)
(SEQ ID NO: 3)
ACGAGAGTTTGATCCTGGCTCAGGATGAACGCTGGCGGCGTGCCTAACACATGCAAGTCGAGCGAAGCGCTGTTT
TCAGAATCTTCGGAGGAAGAGGACAGTGACTGAGCGGCGGACGGGTGAGTAACGCGTGGGCAACCTGCCTCATAC
AGGGGGATAACAGTTAGAAATGACTGCTAATACCGCATAAGCGCACAGGACCGCATGGTGTAGTGTGAAAAACTC
CGGTGGTATGAGATGGACCCGCGTCTGATTAGGTAGTTGGTGGGGTAAAGGCCTACCAAGCCGACGATCAGTAGC
CGACCTGAGAGGGTGACCGGCCACATTGGGACTGAGACACGGCCCAAACTCCTACGGGAGGCAGCAGTGGGGAAT
ATTGCACAATGGGGGAAACCCTGATGCAGCGACGCCGCGTGAAGGAAGAAGTATTTCGGTATGTAAACTTCTATC
AGCAGGGAAGAAGATGACGGTACCTGAGTAAGAAGCACCGGCTAAATACGTGCCAGCAGCCGCGGTAATACGTAT
GGTGCAAGCGTTATCCGGATTTACTGGGTGTAAAGGGAGCGTAGACGGATAGGCAAGTCTGGAGTGAAAACCCAG
GGCTCAACCCTGGGACTGCTTTGGAAACTGCAGATCTGGAGTGCCGGAGAGGTAAGCGGAATTCCTAGTGTAGCG
GTGAAATGCGTAGATATTAGGAGGAACACCAGTGGCGAAGGCGGCTTACTGGACGGTGACTGACGTTGAGGCTCG
AAAGCGTGGGGAGCAAACAGGATTAGATACCCTGGTAGTCCACGCCGTAAACGATGACTACTAGGTGTCGGTGTG
CAAAGCACATCGGTGCCGCAGCAAACGCAATAAGTAGTCCACCTGGGGAGTACGTTCGCAAGAATGAAACTCAAA
GGAATTGACGGGGACCCGCACAAGCGGTGGAGCATGTGGTTTAATTCGAAGCAACGCGAAGAACCTTACCTGGTC
TTGACATCCGGATGACGGGCGAGTAATGTCGCCGTCCCTTCGGGGCATCCGAGACAGGTGGTGCATGGTTGTCGT
CAGCTCGTGTCGTGAGATGTTGGGTTAAGTCCCGCAACGAGCGCAACCCTTATCTTCAGTAGCCAGCATATAAGG
TGGGCACTCTGGAGAGACTGCCAGGGAGAACCTGGAGGAAGGTGGGGATGACGTCAAATCATCATGCCCCTTATG
GCCAGGGCTACACACGTGCTACAATGGCGTAAACAAAGGGAAGCGAGAGGGTGACCTGGAGCGAATCCCAAAAAT
AACGTCTCAGTTCGGATTGTAGTCTGCAACTCGACTACATGAAGCTGGAATCGCTAGTAATCGCGGATCAGCATG
CCGCGGTGAATACGTTCCCGGGTCTTGTACACACCGCCCGTCACACCATGGGAGTCAGTAACGCCCGAAGCCAGT
GACCCAACCTTAGAGGAGGGAGCTGTCGAAGGCGGGACGGATAACTGGGGTGAAGTCGTAACAAGGTAGCCGTAT
CGGAAGGTGCGGCTGGATCACCTCCTTT
Strain 4 (VE303-4)
Clostridium symbiosum 16S ribosomal RNA coding sequence (16S
rDNA)
(SEQ ID NO: 4)
ATGAGAGTTTGATCCTGGCTCAGGATGAACGCTGGCGGCGTGCCTAACACATGCAAGTCGAACGAAGCGATTTAA
CGGAAGTTTTCGGATGGAAGTTGGATTGACTGAGTGGCGGACGGGTGAGTAACGCGTGGGTAACCTGCCTTGTAC
TGGGGGACAACAGTTAGAAATGACTGCTAATACCGCATAAGCGCACAGTATCGCATGATACAGTGTGAAAAACTC
CGGTGGTACAAGATGGACCCGCGTCTGATTAGCTAGTTGGTAAGGTAACGGCTTACCAAGGCGACGATCAGTAGC
CGACCTGAGAGGGTGACCGGCCACATTGGGACTGAGACACGGCCCAAACTCCTACGGGAGGCAGCAGTGGGGAAT
ATTGCACAATGGGCGAAAGCCTGATGCAGCGACGCCGCGTGAGTGAAGAAGTATTTCGGTATGTAAAGCTCTATC
AGCAGGGAAGAAAATGACGGTACCTGACTAAGAAGCCCCGGCTAACTACGTGCCAGCAGCCGCGGTAATACGTAG
GGGGCAAGCGTTATCCGGATTTACTGGGTGTAAAGGGAGCGTAGACGGTAAAGCAAGTCTGAAGTGAAAGCCCGC
GGCTCAACTGCGGGACTGCTTTGGAAACTGTTTAACTGGAGTGTCGGAGAGGTAAGTGGAATTCCTAGTGTAGCG
GTGAAATGCGTAGATATTAGGAGGAACACCAGTGGCGAAGGCGACTTACTGGACGATAACTGACGTTGAGGCTCG
AAAGCGTGGGGAGCAAACAGGATTAGATACCCTGGTAGTCCACGCCGTAAACGATGAATACTAGGTGTTGGGGAG
CAAAGCTCTTCGGTGCCGTCGCAAACGCAGTAAGTATTCCACCTGGGGAGTACGTTCGCAAGAATGAAACTCAAA
GGAATTGACGGGGACCCGCACAAGCGGTGGAGCATGTGGTTTAATTCGAAGCAACGCGAAGAACCTTACCAGGTC
TTGACATCGATCCGACGGGGGAGTAACGTCCCCTTCCCTTCGGGGCGGAGAAGACAGGTGGTGCATGGTTGTCGT
CAGCTCGTGTCGTGAGATGTTGGGTTAAGTCCCGCAACGAGCGCAACCCTTATTCTAAGTAGCCAGCGGTTCGGC
CGGGAACTCTTGGGAGACTGCCAGGGATAACCTGGAGGAAGGTGGGGATGACGTCAAATCATCATGCCCCTTATG
ATCTGGGCTACACACGTGCTACAATGGCGTAAACAAAGAGAAGCAAGACCGCGAGGTGGAGCAAATCTCAAAAAT
AACGTCTCAGTTCGGACTGCAGGCTGCAACTCGCCTGCACGAAGCTGGAATCGCTAGTAATCGCGAATCAGAATG
TCGCGGTGAATACGTTCCCGGGTCTTGTACACACCGCCCGTCACACCATGGGAGTCAGTAACGCCCGAAGTCAGT
GACCCAACCGCAAGGAGGGAGCTGCCGAAGGCGGGACCGATAACTGGGGTGAAGTCGTAACAAGGTAGCCGTATC
GGAAGGTGCGGCTGGATCACCTCCTTT
Strain 5 (VE303-5)
Blautia producta 16S ribosomal RNA coding sequence (16S rDNA)
(SEQ ID NO: 5)
TCAGAGAGTTTGATCCTGGCTCAGGATGAACGCTGGCGGCGTGCTTAACACATGCAAGTCGAGCGAAGCACTTAA
GTGGATCTCTTCGGATTGAAACTTATTTGACTGAGCGGCGGACGGGTGAGTAACGCGTGGGTAACCTGCCTCATA
CAGGGGGATAACAGTTAGAAATGGCTGCTAATACCGCATAAGCGCACAGGACCGCATGGTCTGGTGTGAAAAACT
CCGGTGGTATGAGATGGACCCGCGTCTGATTAGCTAGTTGGAGGGGTAACGGCCCACCAAGGCGACGATCAGTAG
CCGGCCTGAGAGGGTGAACGGCCACATTGGGACTGAGACACGGCCCAGACTCCTACGGGAGGCAGCAGTGGGGAA
TATTGCACAATGGGGGAAACCCTGATGCAGCGACGCCGCGTGAAGGAAGAAGTATCTCGGTATGTAAACTTCTAT
CAGCAGGGAAGAAAATGACGGTACCTGACTAAGAAGCCCCGGCTAACTACGTGCCAGCAGCCGCGGTAATACGTA
GGGGGCAAGCGTTATCCGGATTTACTGGGTGTAAAGGGAGCGTAGACGGAAGAGCAAGTCTGATGTGAAAGGCTG
GGGCTTAACCCCAGGACTGCATTGGAAACTGTTTTTCTAGAGTGCCGGAGAGGTAAGCGGAATTCCTAGTGTAGC
GGTGAAATGCGTAGATATTAGGAGGAACATCAGTGGCGAAGGCGGCTTACTGGACGGTAACTGACGTTGAGGCTC
GAAAGCGTGGGGAGCAAACAGGATTAGATACCCTGGTAGTCCACGCCGTAAACGATGAATACTAGGTGTCGGGTG
GCAAAGCCATTCGGTGCCGCAGCAAACGCAATAAGTATTCCACCTGGGGAGTACGTTCGCAAGAATGAAACTCAA
AGGAATTGACGGGGACCCGCACAAGCGGTGGAGCATGTGGTTTAATTCGAAGCAACGCGAAGAACCTTACCAAGT
CTTGACATCCCTCTGACCGGCCCGTAACGGGGCCTTCCCTTCGGGGCAGAGGAGACAGGTGGTGCATGGTTGTCG
TCAGCTCGTGTCGTGAGATGTTGGGTTAAGTCCCGCAACGAGCGCAACCCCTATCCTTAGTAGCCAGCAGGTGAA
GCTGGGCACTCTAGGGAGACTGCCGGGGATAACCCGGAGGAAGGCGGGGACGACGTCAAATCATCATGCCCCTTA
TGATTTGGGCTACACACGTGCTACAATGGCGTAAACAAAGGGAAGCGAGACAGCGATGTTGAGCAAATCCCAAAA
ATAACGTCCTAGTTCGGACTGCAGTCTGCAACTCGACTGCACGAAGCTGGAATCGCTAGTAATCGCGAATCAGAA
TGTCGCGGTGAATACGTTCCCGGGTCTTGTACACACCGCCCGTCACACCATGGGAGTCAGTAACGCCCGAAGTCA
GTGACCCAACCTTACAGGAGGGAGCTGCCGAAGGCGGGACCGATAACTGGGGTGAAGTCGTAACAAGGTAGCCGT
ATCGGAAGGTGCGGCTGGATCACCTCCTTT
Strain 6 (VE303-6) Dorea longicatena 16S ribosomal RNA coding sequence
(16S rDNA)
(SEQ ID NO: 6)
AACGAGAGTTTGATCCTGGCTCAGGATGAACGCTGGCGGCGTGCTTAACACATGCAAGTCGAGCGAAGCACTTTG
GAAAGATTCTTCGGATGATTTCCTTTGTGACTGAGCGGCGGACGGGTGAGTAACGCGTGGGTAACCTGCCTCATA
CAGGGGGATAACAGTTAGAAATGACTGCTAATACCGCATAAGACCACGGTACCGCATGGTACAGTGGTAAAAACT
CCGGTGGTATGAGATGGACCCGCGTCTGATTAGGTAGTTGGTGGGGTAACGGCCTACCAAGCCGACGATCAGTAG
CCGACCTGAGAGGGTGACCGGCCACATTGGGACTGAGACACGGCCCAGACTCCTACGGGAGGCAGCAGTGGGGAA
TATTGCACAATGGAGGAAACTCTGATGCAGCGACGCCGCGTGAAGGATGAAGTATTTCGGTATGTAAACTTCTAT
CAGCAGGGAAGAAAATGACGGTACCTGACTAAGAAGCCCCGGCTAACTACGTGCCAGCAGCCGCGGTAATACGTA
GGGGGCAAGCGTTATCCGGATTTACTGGGTGTAAAGGGAGCGTAGACGGCACGGCAAGCCAGATGTGAAAGCCCG
GGGCTCAACCCCGGGACTGCATTTGGAACTGCTGAGCTAGAGTGTCGGAGAGGCAAGTGGAATTCCTAGTGTAGC
GGTGAAATGCGTAGATATTAGGAGGAACACCAGTGGCGAAGGCGGCTTGCTGGACGATGACTGACGTTGAGGCTC
GAAAGCGTGGGGAGCAAACAGGATTAGATACCCTGGTAGTCCACGCCGTAAACGATGACTGCTAGGTGTCGGGTG
GCAAAGCCATTCGGTGCCGCAGCTAACGCAATAAGCAGTCCACCTGGGGAGTACGTTCGCAAGAATGAAACTCAA
AGGAATTGACGGGGACCCGCACAAGCGGTGGAGCATGTGGTTTAATTCGAAGCAACGCGAAGAACCTTACCTGAT
CTTGACATCCCGATGACCGCTTCGTAATGGAAGCTTTTCTTCGGAACATCGGTGACAGGTGGTGCATGGTTGTCG
TCAGCTCGTGTCGTGAGATGTTGGGTTAAGTCCCGCAACGAGCGCAACCCCTATCTTCAGTAGCCAGCAGGTTAA
GCTGGGCACTCTGGAGAGACTGCCAGGGATAACCTGGAGGAAGGTGGGGATGACGTCAAATCATCATGCCCCTTA
TGACCAGGGCTACACACGTGCTACAATGGCGTAAACAAAGAGAAGCGAACTCGCGAGGGTAAGCAAATCTCAAAA
ATAACGTCTCAGTTCGGATTGTAGTCTGCAACTCGACTACATGAAGCTGGAATCGCTAGTAATCGCAGATCAGAA
TGCTGCGGTGAATACGTTCCCGGGTCTTGTACACACCGCCCGTCACACCATGGGAGTCAGTAACGCCCGAAGTCA
GTGACCCAACCGTAAGGAGGGAGCTGCCGAAGGTGGGACCGATAACTGGGGTGAAGTCGTAACAAGGTAGCCGTA
TCGGAAGGTGCGGCTGGATCACCTCCTTT
Strain 7 (VE303-7)
Clostridium innocuum 16S ribosomal RNA coding sequence (16S rDNA)
(SEQ ID NO: 7)
ATGGAGAGTTTGATCCTGGCTCAGGATGAACGCTGGCGGCATGCCTAATACATGCAAGTCGAACGAAGTTTCGAG
GAAGCTTGCTTCCAAAGAGACTTAGTGGCGAACGGGTGAGTAACACGTAGGTAACCTGCCCATGTGTCCGGGATA
ACTGCTGGAAACGGTAGCTAAAACCGGATAGGTATACAGAGCGCATGCTCAGTATATTAAAGCGCCCATCAAGGC
GTGAACATGGATGGACCTGCGGCGCATTAGCTAGTTGGTGAGGTAACGGCCCACCAAGGCGATGATGCGTAGCCG
GCCTGAGAGGGTAAACGGCCACATTGGGACTGAGACACGGCCCAAACTCCTACGGGAGGCAGCAGTAGGGAATTT
TCGTCAATGGGGGAAACCCTGAACGAGCAATGCCGCGTGAGTGAAGAAGGTCTTCGGATCGTAAAGCTCTGTTGT
AAGTGAAGAACGGCTCATAGAGGAAATGCTATGGGAGTGACGGTAGCTTACCAGAAAGCCACGGCTAACTACGTG
CCAGCAGCCGCGGTAATACGTAGGTGGCAAGCGTTATCCGGAATCATTGGGCGTAAAGGGTGCGTAGGTGGCGTA
CTAAGTCTGTAGTAAAAGGCAATGGCTCAACCATTGTAAGCTATGGAAACTGGTATGCTGGAGTGCAGAAGAGGG
CGATGGAATTCCATGTGTAGCGGTAAAATGCGTAGATATATGGAGGAACACCAGTGGCGAAGGCGGTCGCCTGGT
CTGTAACTGACACTGAGGCACGAAAGCGTGGGGAGCAAATAGGATTAGATACCCTAGTAGTCCACGCCGTAAACG
ATGAGAACTAAGTGTTGGAGGAATTCAGTGCTGCAGTTAACGCAATAAGTTCTCCGCCTGGGGAGTATGCACGCA
AGTGTGAAACTCAAAGGAATTGACGGGGGCCCGCACAAGCGGTGGAGTATGTGGTTTAATTCGAAGCAACGCGAA
GAACCTTACCAGGCCTTGACATGGAAACAAATACCCTAGAGATAGGGGGATAATTATGGATCACACAGGTGGTGC
ATGGTTGTCGTCAGCTCGTGTCGTGAGATGTTGGGTTAAGTCCCGCAACGAGCGCAACCCTTGTCGCATGTTACC
AGCATCAAGTTGGGGACTCATGCGAGACTGCCGGTGACAAACCGGAGGAAGGTGGGGATGACGTCAAATCATCAT
GCCCCTTATGGCCTGGGCTACACACGTACTACAATGGCGACCACAAAGAGCAGCGACACAGTGATGTGAAGCGAA
TCTCATAAAGGTCGTCTCAGTTCGGATTGAAGTCTGCAACTCGACTTCATGAAGTCGGAATCGCTAGTAATCGCA
GATCAGCATGCTGCGGTGAATACGTTCTCGGGCCTTGTACACACCGCCCGTCAAACCATGGGAGTCAGTAATACC
CGAAGCCGGTGGCATAACCGCAAGGAGTGAGCCGTCGAAGGTAGGACCGATGACTGGGGTTAAGTCGTAACAAGG
TATCCCTACGGGAACGTGGGGATGGATCACCTCCTTT
Strain 8 (VE303-8)
Flavonifractor plautii 16S ribosomal RNA coding sequence (16S rDNA)
(SEQ ID NO: 8)
TATTGAGAGTTTGATCCTGGCTCAGGATGAACGCTGGCGGCGTGCTTAACACATGCAAGTCGAACGGGGTGCTCA
TGACGGAGGATTCGTCCAACGGATTGAGTTACCTAGTGGCGGACGGGTGAGTAACGCGTGAGGAACCTGCCTTGG
AGAGGGGAATAACACTCCGAAAGGAGTGCTAATACCGCATGATGCAGTTGGGTCGCATGGCTCTGACTGCCAAAG
ATTTATCGCTCTGAGATGGCCTCGCGTCTGATTAGCTAGTAGGCGGGGTAACGGCCCACCTAGGCGACGATCAGT
AGCCGGACTGAGAGGTTGACCGGCCACATTGGGACTGAGACACGGCCCAGACTCCTACGGGAGGCAGCAGTGGGG
AATATTGGGCAATGGGCGCAAGCCTGACCCAGCAACGCCGCGTGAAGGAAGAAGGCTTTCGGGTTGTAAACTTCT
TTTGTCGGGGACGAAACAAATGACGGTACCCGACGAATAAGCCACGGCTAACTACGTGCCAGCAGCCGCGGTAAT
ACGTAGGTGGCAAGCGTTATCCGGATTTACTGGGTGTAAAGGGCGTGTAGGCGGGATTGCAAGTCAGATGTGAAA
ACTGGGGGCTCAACCTCCAGCCTGCATTTGAAACTGTAGTTCTTGAGTGCTGGAGAGGCAATCGGAATTCCGTGT
GTAGCGGTGAAATGCGTAGATATACGGAGGAACACCAGTGGCGAAGGCGGATTGCTGGACAGTAACTGACGCTGA
GGCGCGAAAGCGTGGGGAGCAAACAGGATTAGATACCCTGGTAGTCCACGCCGTAAACGATGGATACTAGGTGTG
GGGGGTCTGACCCCCTCCGTGCCGCAGTTAACACAATAAGTATCCCACCTGGGGAGTACGATCGCAAGGTTGAAA
CTCAAAGGAATTGACGGGGGCCCGCACAAGCGGTGGAGTATGTGGTTTAATTCGAAGCAACGCGAAGAACCTTAC
CAGGGCTTGACATCCCACTAACGAAGCAGAGATGCATTAGGTGCCCTTCGGGGAAAGTGGAGACAGGTGGTGCAT
GGTTGTCGTCAGCTCGTGTCGTGAGATGTTGGGTTAAGTCCCGCAACGAGCGCAACCCTTATTGTTAGTTGCTAC
GCAAGAGCACTCTAGCGAGACTGCCGTTGACAAAACGGAGGAAGGTGGGGACGACGTCAAATCATCATGCCCCTT
ATGTCCTGGGCCACACACGTACTACAATGGTGGTTAACAGAGGGAGGCAATACCGCGAGGTGGAGCAAATCCCTA
AAAGCCATCCCAGTTCGGATTGCAGGCTGAAACCCGCCTGTATGAAGTTGGAATCGCTAGTAATCGCGGATCAGC
ATGCCGCGGTGAATACGTTCCCGGGCCTTGTACACACCGCCCGTCACACCATGAGAGTCGGGAACACCCGAAGTC
CGTAGCCTAACCGCAAGGAGGGCGCGGCCGAAGGTGGGTTCGATAATTGGGGTGAAGTCGTAACAAGGTAGCCGT
ATCGGAAGGTGCGGCTGGATCACCTCCTTT
Strain 9
Lachnospiraceae bacterium 16S ribosomal RNA coding sequence (16S rDNA)
(SEQ ID NO: 9)
TATTGAGAGTTTGATCCTGGCTCAGGATGAACGCTGGCGGCGTGCTTAACACATGCAAGTCGAACGGGGTGCTCA
TGACGGAGGATTCGTCCAACGGATTGAGTTACCTAGTGGCGGACGGGTGAGTAACGCGTGAGGAACCTGCCTTGG
AGAGGGGAATAACACTCCGAAAGGAGTGCTAATACCGCATGATGCAGTTGGGTCGCATGGCTCTGACTGCCAAAG
ATTTATCGCTCTGAGATGGCCTCGCGTCTGATTAGCTAGTAGGCGGGGTAACGGCCCACCTAGGCGACGATCAGT
AGCCGGACTGAGAGGTTGACCGGCCACATTGGGACTGAGACACGGCCCAGACTCCTACGGGAGGCAGCAGTGGGG
AATATTGGGCAATGGGCGCAAGCCTGACCCAGCAACGCCGCGTGAAGGAAGAAGGCTTTCGGGTTGTAAACTTCT
TTTGTCGGGGACGAAACAAATGACGGTACCCGACGAATAAGCCACGGCTAACTACGTGCCAGCAGCCGCGGTAAT
ACGTAGGTGGCAAGCGTTATCCGGATTTACTGGGTGTAAAGGGCGTGTAGGCGGGATTGCAAGTCAGATGTGAAA
ACTGGGGGCTCAACCTCCAGCCTGCATTTGAAACTGTAGTTCTTGAGTGCTGGAGAGGCAATCGGAATTCCGTGT
GTAGCGGTGAAATGCGTAGATATACGGAGGAACACCAGTGGCGAAGGCGGATTGCTGGACAGTAACTGACGCTGA
GGCGCGAAAGCGTGGGGAGCAAACAGGATTAGATACCCTGGTAGTCCACGCCGTAAACGATGGATACTAGGTGTG
GGGGGTCTGACCCCCTCCGTGCCGCAGTTAACACAATAAGTATCCCACCTGGGGAGTACGATCGCAAGGTTGAAA
CTCAAAGGAATTGACGGGGGCCCGCACAAGCGGTGGAGTATGTGGTTTAATTCGAAGCAACGCGAAGAACCTTAC
CAGGGCTTGACATCCCACTAACGAAGCAGAGATGCATTAGGTGCCCTTCGGGGAAAGTGGAGACAGGTGGTGCAT
GGTTGTCGTCAGCTCGTGTCGTGAGATGTTGGGTTAAGTCCCGCAACGAGCGCAACCCTTATTGTTAGTTGCTAC
GCAAGAGCACTCTAGCGAGACTGCCGTTGACAAAACGGAGGAAGGTGGGGACGACGTCAAATCATCATGCCCCTT
ATGTCCTGGGCCACACACGTACTACAATGGTGGTTAACAGAGGGAGGCAATACCGCGAGGTGGAGCAAATCCCTA
AAAGCCATCCCAGTTCGGATTGCAGGCTGAAACCCGCCTGTATGAAGTTGGAATCGCTAGTAATCGCGGATCAGC
ATGCCGCGGTGAATACGTTCCCGGGCCTTGTACACACCGCCCGTCACACCATGAGAGTCGGGAACACCCGAAGTC
CGTAGCCTAACCGCAAGGAGGGCGCGGCCGAAGGTGGGTTCGATAATTGGGGTGAAGTCGTAACAAGGTAGCCGT
ATCGGAAGGTGCGGCTGGATCACCTCCTTT
Strain 10
Bifidobacterium longum 16S ribosomal RNA coding sequence (16S rDNA)
(SEQ ID NO: 10)
TTTGTGGAGGGTTCGATTCTGGCTCAGGATGAACGCTGGCGGCGTGCTTAACACATGCAAGTCGAACGGGATCCA
TCAAGCTTGCTTGGTGGTGAGAGTGGCGAACGGGTGAGTAATGCGTGACCGACCTGCCCCATACACCGGAATAGC
TCCTGGAAACGGGTGGTAATGCCGGATGTTCCAGTTGATCGCATGGTCTTCTGGGAAAGCTTTCGCGGTATGGGA
TGGGGTCGCGTCCTATCAGCTTGACGGCGGGGTAACGGCCCACCGTGGCTTCGACGGGTAGCCGGCCTGAGAGGG
CGACCGGCCACATTGGGACTGAGATACGGCCCAGACTCCTACGGGAGGCAGCAGTGGGGAATATTGCACAATGGG
CGCAAGCCTGATGCAGCGACGCCGCGTGAGGGATGGAGGCCTTCGGGTTGTAAACCTCTTTTATCGGGGAGCAAG
CGAGAGTGAGTTTACCCGTTGAATAAGCACCGGCTAACTACGTGCCAGCAGCCGCGGTAATACGTAGGGTGCAAG
CGTTATCCGGAATTATTGGGCGTAAAGGGCTCGTAGGCGGTTCGTCGCGTCCGGTGTGAAAGTCCATCGCTTAAC
GGTGGATCCGCGCCGGGTACGGGCGGGCTTGAGTGCGGTAGGGGAGACTGGAATTCCCGGTGTAACGGTGGAATG
TGTAGATATCGGGAAGAACACCAATGGCGAAGGCAGGTCTCTGGGCCGTTACTGACGCTGAGGAGCGAAAGCGTG
GGGAGCGAACAGGATTAGATACCCTGGTAGTCCACGCCGTAAACGGTGGATGCTGGATGTGGGGCCCGTTCCACG
GGTTCCGTGTCGGAGCTAACGCGTTAAGCATCCCGCCTGGGGAGTACGGCCGCAAGGCTAAAACTCAAAGAAATT
GACGGGGGCCCGCACAAGCGGCGGAGCATGCGGATTAATTCGATGCAACGCGAAGAACCTTACCTGGGCTTGACA
TGTTCCCGACGGTCGTAGAGATACGGCTTCCCTTCGGGGGGGGTTCACAGGTGGTGCATGGTCGTCGTCAGCTCG
TGTCGTGAGATGTTGGGTTAAGTCCCGCAACGAGCGCAACCCTCGCCCCGTGTTGCCAGCGGATTATGCCGGGAA
CTCACGGGGGACCGCCGGGGTTAACTCGGAGGAAGGTGGGGATGACGTCAGATCATCATGCCCCTTACGTCCAGG
GCTTCACGCATGCTACAATGGCCGGTACAACGGGATGCGACGCGGCGACGCGGAGCGGATCCCTGAAAACCGGTC
TCAGTTCGGATCGCAGTCTGCAACTCGACTGCGTGAAGGCGGAGTCGCTAGTAATCGCGAATCAGCAACGTCGCG
GTGAATGCGTTCCCGGGCCTTGTACACACCGCCCGTCAAGTCATGAAAGTGGGCAGCACCCGAAGCCGGTGGCCT
AACCCCTTGTGGGATGGAGCCGTCTAAGGTGAGGCTCGTGATTGGGACTAAGTCGTAACAAGGTAGCCGTACCGG
AAGGTGCGGCTGGATCACCTCCTTT

### ENUMERATED EMBODIMENTS

1. A composition comprising a purified bacterial mixture, the purified bacterial mixture comprising one or more bacterial strains belonging to species independently selected from Clostridium bolteae, Anaerotruncus colihominis, and Flavonifractor plautii, wherein the purified bacterial mixture:
   (a) does not comprise a bacterial strain belonging to the species Sellimonas intestinalis;
   (b) does not comprise a bacterial strain belonging to the species Clostridium symbiosum;
   (c) does not comprise a bacterial strain belonging to the species Blautia producta;
   (d) does not comprise a bacterial strain belonging to the species Dorea longicatena and does not comprise a bacterial strain belonging to the species Clostridium innocuum;
   (e) further comprises a bacterial strain belonging to the species Lachnospiraceae bacterium; and/or
   (f) further comprises a bacterial strain belonging to the species Bifidobacterium longum.
2. A combination product comprising one or more bacterial strains belonging to species independently selected from Clostridium bolteae, Anaerotruncus colihominis, and Flavonifractor plautii, wherein the combination product:
   (a) does not comprise a bacterial strain belonging to the species Sellimonas intestinalis;
   (b) does not comprise a bacterial strain belonging to the species Clostridium symbiosum;
   (c) does not comprise a bacterial strain belonging to the species Blautia producta;
   (d) does not comprise a bacterial strain belonging to the species Dorea longicatena and does not comprise a bacterial strain belonging to the species Clostridium innocuum;
   (e) further comprises a bacterial strain belonging to the species Lachnospiraceae bacterium; and/or
   (f) further comprises a bacterial strain belonging to the species Bifidobacterium longum.
3. A kit comprising one or more bacterial strains belonging to species independently selected from Clostridium bolteae, Anaerotruncus colihominis, and Flavonifractor plautii, wherein the kit:
   (a) does not comprise a bacterial strain belonging to the species Sellimonas intestinalis;
   (b) does not comprise a bacterial strain belonging to the species Clostridium symbiosum;
   (c) does not comprise a bacterial strain belonging to the species Blautia producta;
   (d) does not comprise a bacterial strain belonging to the species Dorea longicatena and does not comprise a bacterial strain belonging to the species Clostridium innocuum;
   (e) further comprises a bacterial strain belonging to the species Lachnospiraceae bacterium; and/or
   (f) further comprises a bacterial strain belonging to the species Bifidobacterium longum,
   wherein the bacterial strains are comprised in one or more dosage forms, each dosage form comprising at least one of the bacterial strains.
4. A composition comprising a purified bacterial mixture, the purified bacterial mixture comprising one or more of:
   (a) a bacterial strain comprising a 16S rDNA sequence with at least 97% sequence identity to the nucleotide sequence of SEQ ID NO: 1;
   (b) a bacterial strain comprising a 16S rDNA sequence with at least 97% sequence identity to the nucleotide sequence of SEQ ID NO: 2; and
   (c) a bacterial strain comprising a 16S rDNA sequence with at least 97% sequence identity to the nucleotide sequence of SEQ ID NO: 8,
wherein the purified bacterial mixture:
   (d) does not comprise a bacterial strain comprising a 16S rDNA sequence with at least 97% sequence identity to the nucleotide sequence of SEQ ID NO: 3;
   (e) does not comprise a bacterial strain comprising a 16S rDNA sequence with at least 97% sequence identity to the nucleotide sequence of SEQ ID NO: 4;
   (f) does not comprise a bacterial strain comprising a 16S rDNA sequence with at least 97% sequence identity to the nucleotide sequence of SEQ ID NO: 5;
   (g) does not comprise a bacterial strain comprising a 16S rDNA sequence with at least 97% sequence identity to the nucleotide sequence of SEQ ID NO: 6, and does not comprise a bacterial strain comprising a 16S rDNA sequence with at least 97% sequence identity to the nucleotide sequence of SEQ ID NO: 7;
   (h) further comprises a bacterial strain comprising a 16S rDNA sequence with at least 97% sequence identity to the nucleotide sequence of SEQ ID NO: 9; and/or
   (i) further comprises a bacterial strain comprising a 16S rDNA sequence with at least 97% sequence identity to the nucleotide sequence of SEQ ID NO: 10.
5. A combination product comprising one or more of:
   (a) a bacterial strain comprising a 16S rDNA sequence with at least 97% sequence identity to the nucleotide sequence of SEQ ID NO: 1;
   (b) a bacterial strain comprising a 16S rDNA sequence with at least 97% sequence identity to the nucleotide sequence of SEQ ID NO: 2; and
   (c) a bacterial strain comprising a 16S rDNA sequence with at least 97% sequence identity to the nucleotide sequence of SEQ ID NO: 8,
wherein the combination product:
   (d) does not comprise a bacterial strain comprising a 16S rDNA sequence with at least 97% sequence identity to the nucleotide sequence of SEQ ID NO: 3;
   (e) does not comprise a bacterial strain comprising a 16S rDNA sequence with at least 97% sequence identity to the nucleotide sequence of SEQ ID NO: 4;
   (f) does not comprise a bacterial strain comprising a 16S rDNA sequence with at least 97% sequence identity to the nucleotide sequence of SEQ ID NO: 5;
   (g) does not comprise a bacterial strain comprising a 16S rDNA sequence with at least 97% sequence identity to the nucleotide sequence of SEQ ID NO: 6, and does not comprise a bacterial strain comprising a 16S rDNA sequence with at least 97% sequence identity to the nucleotide sequence of SEQ ID NO: 7;
   (h) further comprises a bacterial strain comprising a 16S rDNA sequence with at least 97% sequence identity to the nucleotide sequence of SEQ ID NO: 9; and/or
   (i) further comprises a bacterial strain comprising a 16S rDNA sequence with at least 97% sequence identity to the nucleotide sequence of SEQ ID NO: 10.
6. A kit comprising one or more of:
   (a) a bacterial strain comprising a 16S rDNA sequence with at least 97% sequence identity to the nucleotide sequence of SEQ ID NO: 1;
   (b) a bacterial strain comprising a 16S rDNA sequence with at least 97% sequence identity to the nucleotide sequence of SEQ ID NO: 2; and
   (c) a bacterial strain comprising a 16S rDNA sequence with at least 97% sequence identity to the nucleotide sequence of SEQ ID NO: 8,
wherein the kit:
   (d) does not comprise a bacterial strain comprising a 16S rDNA sequence with at least 97% sequence identity to the nucleotide sequence of SEQ ID NO: 3;
   (e) does not comprise a bacterial strain comprising a 16S rDNA sequence with at least 97% sequence identity to the nucleotide sequence of SEQ ID NO: 4;
   (f) does not comprise a bacterial strain comprising a 16S rDNA sequence with at least 97% sequence identity to the nucleotide sequence of SEQ ID NO: 5;
   (g) does not comprise a bacterial strain comprising a 16S rDNA sequence with at least 97% sequence identity to the nucleotide sequence of SEQ ID NO: 6, and does not comprise a bacterial strain comprising a 16S rDNA sequence with at least 97% sequence identity to the nucleotide sequence of SEQ ID NO: 7;
   (h) further comprises a bacterial strain comprising a 16S rDNA sequence with at least 97% sequence identity to the nucleotide sequence of SEQ ID NO: 9; and/or
   (i) further comprises a bacterial strain comprising a 16S rDNA sequence with at least 97% sequence identity to the nucleotide sequence of SEQ ID NO: 10,
   wherein the bacterial strains are comprised in one or more dosage forms, each dosage form comprising at least one of the bacterial strains.
7. The composition of Embodiment 1 or 4, combination product of Embodiment 2 or 5, or kit of Embodiment 3 or 6, wherein:
   (a) the bacterial strain of (a) comprises a 16S rDNA sequence with at least 99% sequence identity to the nucleotide sequence of SEQ ID NO: 1;
   (b) the bacterial strain of (b) comprises a 16S rDNA sequence with at least 99% sequence identity to the nucleotide sequence of SEQ ID NO: 2;
   (c) the bacterial strain of (c) comprises a 16S rDNA sequence with at least 99% sequence identity to the nucleotide sequence of SEQ ID NO: 8;
   (h) the bacterial strain of (h) comprises a 16S rDNA sequence with at least 99% sequence identity to the nucleotide sequence of SEQ ID NO: 9; and/or
   (i) the bacterial strain of (h) comprises a 16S rDNA sequence with at least 99% sequence identity to the nucleotide sequence of SEQ ID NO: 10.
8. The composition of any one of Embodiments 1, 4, and 7, combination product of any one of Embodiments 2, 5, and 7, or kit of any one of Embodiments 3 and 6-7, wherein one or more of the bacterial strains are in vegetative form.
9. The composition of any one of Embodiments 1, 4, 7, and 8, combination product of any one of Embodiments 2, 5, and 7-8, or kit of any one of Embodiments 3 and 6-8, wherein one or more of the bacterial strains are in spore form.
10. The composition of any one of Embodiments 1, 4, and 7-9, combination product of any one of Embodiments 2, 5, and 7-9, or kit of any one of Embodiments 3 and 6-9, wherein the bacterial strains are lyophilized.
11. The composition of any one of Embodiments 1, 4, and 7-9, combination product of any one of Embodiments 2, 5, and 7-9, or kit of any one of Embodiments 3 and 6-9, wherein the bacterial strains are spray-dried.
12. The composition of any one of Embodiments 1, 4, and 7-11, combination product of any one of Embodiments 2, 5, and 7-11, or kit of any one of Embodiments 3 and 6-11, wherein the bacterial strains are comprised in one or more capsules.
13. The composition of any one of Embodiments 1, 4, and 7-12, combination product of any one of Embodiments 2, 5, and 7-12, or kit of any one of Embodiments 3 and 6-12, wherein the bacterial strains are in contact with one or more pH-sensitive compositions comprising enteric polymers.
14. A method of treating or preventing Clostridioides difficile infection (CDI) in a subject, the method comprising administering the composition of any one of Embodiments 1, 4, and 7-13, or the combination product of any one of claims 2, 5, and 7-13, or the dosage forms of any one of claims 3 and 6-13, to a subject in need thereof.
15. The method of Embodiment 14, wherein the CDI is recurrent CDI (rCDI).
16. The method of Embodiment 14 or 15, wherein the administering is oral administration.
17. The method of any one of Embodiments 14-16, further comprising administering an antibiotic to the subject before the composition, combination product, or dosage forms.
18. The method of Embodiment 17, wherein the antibiotic is vancomycin.
19. The method of any one of Embodiments 14-18, wherein the subject is a human.
20. The method of any one of Embodiments 14-19, wherein at least one of the bacterial strains is detected in a fecal sample obtained from the subject at least two weeks after the administering.
21. The method of any one of Embodiments 14-20, further comprising measuring the abundance of one or more of the bacterial strains in a fecal sample obtained from the subject at least two weeks after the administering.
22. The method of any one of Embodiments 14-21, further comprising (i) identifying one or more of the bacterial strains as not having colonized the subject, if they are not detected in a fecal sample obtained from the subject at least two weeks after the administering; and (ii) administering one or more additional doses of the bacterial strains identified as not having colonized the subject.
A1. A composition comprising a purified bacterial mixture, wherein the purified bacterial mixture:
   (i) comprises two or more bacterial strains belonging to species independently selected from Clostridium bolteae, Anaerotruncus colihominis, and Flavonifractor plautii; and
   (ii) does not comprise bacterial strains belonging to two or more species independently selected from Sellimonas inteestinalis, Clostridium symbiosum, Blautia producta, Dorea longicatena, and Clostridium innocuum.
A2. The composition of Embodiment A1, wherein the purified bacterial mixture does not comprise a bacterial strain belonging to Dorea longicatena, and does not comprise a bacterial strain belonging to Clostridium innocuum.
A3. The composition of any one of the preceding Embodiments, wherein the purified bacterial mixture:
   (a) does not comprise a bacterial strain belonging to Sellimonas inteestinalis;
   (b) does not comprise a bacterial strain belonging to Clostridium symbiosum;
   (c) does not comprise a bacterial strain belonging to Blautia producta;
   (d) does not comprise a bacterial strain belonging to Dorea longicatena, and
   (e) does not comprise a bacterial strain belonging to Clostridium innocuum.
A4. The composition of any one of Embodiments A1-A3, wherein the purified bacterial mixture comprises Clostridium bolteae and Anaerotruncus colihominis.)
A5. The composition of any one of Embodiments A1-A3, wherein the purified bacterial mixture comprises Clostridium bolteae and Flavonifractor plautii.
A6. The composition of any one of Embodiments A1-A3, wherein the purified bacterial mixture comprises Anaerotruncus colihominis and Flavonifractor plautii.
A7. The composition of any one of Embodiments A1-A3, wherein the purified bacterial mixture comprises Clostridium bolteae, Anaerotruncus colihominis, and Flavonifractor plautii.
A8. The composition of any one of Embodiments A1-A3, wherein the purified bacterial mixture consists of Clostridium bolteae and Anaerotruncus colihominis.
A9. The composition of any one of Embodiments A1-A3, wherein the purified bacterial mixture consists of Clostridium bolteae and Flavonifractor plautii.
A10. The composition of any one of Embodiments A1-A3, wherein the purified bacterial mixture consists of Anaerotruncus colihominis and Flavonifractor plautii.
A11. The composition of any one of Embodiments A1-A3, wherein the purified bacterial mixture consists of Clostridium bolteae, Anaerotruncus colihominis, and Flavonifractor plautii.
A12. A composition comprising a purified bacterial mixture, wherein the purified bacterial mixture:
   (i) comprises two or more bacterial strains, each comprising a 16S rDNA sequence having at least 97% sequence identity to a nucleic acid sequence independently selected from SEQ ID NOs: 1, 2, and 8; and
   (ii) does not comprise two or more bacterial strains independently selected from:
      (a) a bacterial strain comprising a 16S rDNA sequence having at least 97% sequence identity to the nucleic acid sequence of SEQ ID NO: 3;
      (b) a bacterial strain comprising a 16S rDNA sequence having at least 97% sequence identity to the nucleic acid sequence of SEQ ID NO: 4;
      (c) a bacterial strain comprising a 16S rDNA sequence having at least 97% sequence identity to the nucleic acid sequence of SEQ ID NO: 5;
      (d) a bacterial strain comprising a 16S rDNA sequence having at least 97% sequence identity to the nucleic acid sequence of SEQ ID NO: 6; and
      (e) a bacterial strain comprising a 16S rDNA sequence having at least 97% sequence identity to the nucleic acid sequence of SEQ ID NO: 7.
A13. The composition of Embodiment A12, wherein the purified bacterial mixture does not comprise a bacterial strain comprising a 16S rDNA sequence having at least 97% sequence identity to the nucleic acid sequence of SEQ ID NO: 6, and does not comprise a bacterial strain comprising a 16S rDNA sequence having at least 97% sequence identity to the nucleic acid sequence of SEQ ID NO: 7.
A14. The composition of Embodiment A12 or A13, wherein the purified bacterial mixture:
   (a) does not comprise a bacterial strain comprising a 16S rDNA sequence having at least 97% sequence identity to the nucleic acid sequence of SEQ ID NO: 3;
   (b) does not comprise a bacterial strain comprising a 16S rDNA sequence having at least 97% sequence identity to the nucleic acid sequence of SEQ ID NO: 4;
   (c) does not comprise a bacterial strain comprising a 16S rDNA sequence having at least 97% sequence identity to the nucleic acid sequence of SEQ ID NO: 5;
   (d) does not comprise a bacterial strain comprising a 16S rDNA sequence having at least 97% sequence identity to the nucleic acid sequence of SEQ ID NO: 6; and
   (e) does not comprise a bacterial strain comprising a 16S rDNA sequence having at least 97% sequence identity to the nucleic acid sequence of SEQ ID NO: 7.
A15. The composition of any one of Embodiments A12-A14, wherein the purified bacterial mixture comprises a bacterial strain comprising a 16S rDNA sequence having at least 97% sequence identity to the nucleic acid sequence of SEQ ID NO: 1, and a bacterial strain comprising a 16S rDNA sequence having at least 97% sequence identity to the nucleic acid sequence of SEQ ID NO: 2.
A16. The composition of any one of Embodiments A12-A14, wherein the purified bacterial mixture comprises a bacterial strain comprising a 16S rDNA sequence having at least 97% sequence identity to the nucleic acid sequence of SEQ ID NO: 1, and a bacterial strain comprising a 16S rDNA sequence having at least 97% sequence identity to the nucleic acid sequence of SEQ ID NO: 8.
A17. The composition of any one of Embodiments A12-A14, wherein the purified bacterial mixture comprises a bacterial strain comprising a 16S rDNA sequence having at least 97% sequence identity to the nucleic acid sequence of SEQ ID NO: 2, and a bacterial strain comprising a 16S rDNA sequence having at least 97% sequence identity to the nucleic acid sequence of SEQ ID NO: 8.
A18. The composition of any one of Embodiments A12-A14, wherein the purified bacterial mixture comprises a bacterial strain comprising a 16S rDNA sequence having at least 97% sequence identity to the nucleic acid sequence of SEQ ID NO: 1, a bacterial strain comprising a 16S rDNA sequence having at least 97% sequence identity to the nucleic acid sequence of SEQ ID NO: 2, and a bacterial strain comprising a 16S rDNA sequence having at least 97% sequence identity to the nucleic acid sequence of SEQ ID NO: 8.
A19. The composition of any one of Embodiments A12-A14, wherein the purified bacterial mixture consists of a bacterial strain comprising a 16S rDNA sequence having at least 97% sequence identity to the nucleic acid sequence of SEQ ID NO: 1, and a bacterial strain comprising a 16S rDNA sequence having at least 97% sequence identity to the nucleic acid sequence of SEQ ID NO: 2.
A20. The composition of any one of Embodiments A12-A14, wherein the purified bacterial mixture consists of a bacterial strain comprising a 16S rDNA sequence having at least 97% sequence identity to the nucleic acid sequence of SEQ ID NO: 1, and a bacterial strain comprising a 16S rDNA sequence having at least 97% sequence identity to the nucleic acid sequence of SEQ ID NO: 8.
A21. The composition of any one of Embodiments A12-A14, wherein the purified bacterial mixture consists of a bacterial strain comprising a 16S rDNA sequence having at least 97% sequence identity to the nucleic acid sequence of SEQ ID NO: 2, and a bacterial strain comprising a 16S rDNA sequence having at least 97% sequence identity to the nucleic acid sequence of SEQ ID NO: 8.
A22. The composition of any one of Embodiments A12-A14, wherein the purified bacterial mixture consists of a bacterial strain comprising a 16S rDNA sequence having at least 97% sequence identity to the nucleic acid sequence of SEQ ID NO: 1, a bacterial strain comprising a 16S rDNA sequence having at least 97% sequence identity to the nucleic acid sequence of SEQ ID NO: 2, and a bacterial strain comprising a 16S rDNA sequence having at least 97% sequence identity to the nucleic acid sequence of SEQ ID NO: 8.
B0. The composition of any one of Embodiments A1-A7, wherein the purified bacterial mixture further comprises a bacterial strain belonging to species Lachnospiraceae bacterium and/or a bacterial strain belonging to species Bifidobacterium longum.
B0.1 The composition of Embodiment B0, wherein the purified bacterial mixture comprises Lachnospiraceae bacterium and Bifidobacterium longum.
B0.2. The composition of any one of Embodiments A12-A18, wherein the purified bacterial mixture further comprises a bacterial strain comprising a 16S rDNA sequence having at least 97% sequence identity to the nucleic acid sequence of SEQ ID NO: 10 and/or a bacterial strain comprising a 16S rDNA sequence having at least 97% sequence identity to the nucleic acid sequence of SEQ ID NO: 10.
B0.3. The composition of Embodiment B0.2, wherein the purified bacterial mixture comprises a bacterial strain comprising a 16S rDNA sequence having at least 97% sequence identity to the nucleic acid sequence of SEQ ID NO: 9, and a bacterial strain comprising a 16S rDNA sequence having at least 97% sequence identity to the nucleic acid sequence of SEQ ID NO: 10.
B1. A composition comprising a purified bacterial mixture, wherein the purified bacterial mixture comprises:
   (i) two or more bacterial strains belonging to species independently selected from Clostridium bolteae, Anaerotruncus colihominis, Sellimonas inteestinalis, Clostridium symbiosum, Blautia producta, Dorea longicatena, Clostridium innocuum, and Flavonifractor plautii; and
   (ii) one or more bacterial strains belonging to species independently selected from Lachnospiraceae bacterium and Bifidobacterium longum.
B2. The composition of Embodiment B1, wherein the purified bacterial mixture comprises Clostridium bolteae, Anaerotruncus colihominis, Sellimonas inteestinalis, Clostridium symbiosum, Blautia producta, Dorea longicatena, Clostridium innocuum, and Flavonifractor plautii.
B3. The composition of Embodiment B1 or B2, wherein the purified bacterial mixture comprises Lachnospiraceae bacterium and Bifidobacterium longum.
B4. A composition comprising a purified bacterial mixture, wherein the purified bacterial mixture comprises:
   (i) two or more bacterial strains, each comprising a 16S rDNA sequence having at least 97% sequence identity to a nucleic acid sequence independently selected from SEQ ID NOs: 1-8; and
   (ii) one or more bacterial strains comprising a 16S rDNA sequence having at least 97% sequence identity to a nucleic acid sequence of SEQ ID NO: 9 or SEQ ID NO: 10.
B5. The composition of Embodiment B4, wherein the purified bacterial mixture comprises 8 or more bacterial strains comprising 16S rDNA sequences with at least 97% sequence identity to SEQ ID NOs: 1-8.
B6. The composition of Embodiment B4 or B5, wherein the purified bacterial mixture comprises two or more bacterial strains comprising 16S rDNA sequences with at least 97% sequence identity to SEQ ID NOs: 9-10.
B7. A composition comprising a purified bacterial mixture comprising bacterial strains of species Clostridium bolteae, Anaerotruncus colihominis, Sellimonas inteestinalis, Clostridium symbiosum, Blautia producta, Dorea longicatena, Clostridium innocuum, Flavonifractor plautii, Lachnospiraceae bacterium, and Bifidobacterium longum.
B8. A composition comprising a purified bacterial mixture consisting of bacterial strains of species Clostridium bolteae, Anaerotruncus colihominis, Sellimonas inteestinalis, Clostridium symbiosum, Blautia producta, Dorea longicatena, Clostridium innocuum, Flavonifractor plautii, Lachnospiraceae bacterium, and Bifidobacterium longum.
B9. A composition comprising a purified bacterial mixture comprising ten or more bacterial strains comprising 16S rDNA sequences with at least 97% sequence identity to SEQ ID NOs: 1-10.
B10. A composition comprising a purified bacterial mixture consisting of ten bacterial strains comprising 16S rDNA sequences with at least 97% sequence identity to SEQ ID NOs: 1-10.
C1. The composition of any one of the preceding Embodiments, wherein one or more of the bacterial strains produce a short chain fatty acid (SCFA).
C2. The composition of any one of the preceding Embodiments, wherein one or more of the bacterial strains produce acetate, butyrate, and/or valerate.
C3. The composition of any one of the preceding Embodiments, wherein one or more of the bacterial strains produce a secondary bile acid.
C4. The composition of any one of the preceding Embodiments, wherein one or more of the bacterial strains produce ursodeoxycholic acid (UDCA).
C5. The composition of any one of Embodiments A1-C4, wherein the bacterial strains are lyophilized.
C6. The composition of any one of Embodiments A1-C4, wherein the bacterial strains are spray-dried.
C7. The composition of any one of Embodiments A1-C6, wherein one or more of the bacterial strains are in spore form.
C8. The composition of any one of Embodiments A1-C7, wherein each of the bacterial strains are in spore form.
C9. The composition of any one of Embodiments A1-C7, wherein one or more of the bacterial strains are in vegetative form.
C10. The composition of any one of Embodiments A1-C6 and C9, wherein each of the bacterial strains are in vegetative form.
D1. A pharmaceutical composition comprising the composition of any one of the preceding Embodiments and a pharmaceutically acceptable excipient.
D2. The pharmaceutical composition of Embodiment D1, wherein the pharmaceutical composition is formulated for oral delivery.
D3. The pharmaceutical composition of Embodiment D1, wherein the pharmaceutical composition is formulated for rectal delivery.
D4. The pharmaceutical composition of any one of Embodiments D1-D3, wherein the pharmaceutical composition is formulated for delivery to the intestine.
D5. The pharmaceutical composition of any one of Embodiments D1-D4, wherein the pharmaceutical composition is formulated for delivery to the colon.
D6. The pharmaceutical composition of any one of Embodiments D1-D5, wherein the pharmaceutical composition comprises one or more enteric polymers.
D7. The pharmaceutical composition of any one of Embodiments D1-D6, wherein the pharmaceutical composition is in a capsule.
D8. The pharmaceutical composition of Embodiment D7, wherein the capsule comprises 1×10⁷ to 1×10¹⁰ colony-forming units (CFUs) of the bacterial strains.
E1. A food product comprising the composition of any one of Embodiments A1-C10 and a nutrient.
F1. A method comprising administering the composition of any one of Embodiments A1-D8 or the food product of Embodiment E1 to a subject in need thereof.
F1.1. The method of Embodiment F1, wherein the subject has a pathogenic infection.
F1.2. The method of Embodiment F1 or F1.1, wherein the composition or food product is administered in a therapeutically effective amount to treat a pathogenic infection in the subject.
F2. The method of Embodiment F1.1 or F1.2, wherein the pathogenic infection is an infection with a pathogen selected from the group consisting of Clostridium difficile, Klebsiella, Kluyvera, Veillonella, Enterococci, Enterobacteriaceae, Neisseria gonorrhoeae, Acinetobacter, Campylobacter sp., Pseudomonas aeruginosa, Salmonella sp., Shigella sp., Staphylococcus aureus, Salmonella typhi, Group A Streptococcus, Group B Streptococcus, and Streptococcus pneumoniae.
F3. The method of any one of Embodiments F1.1-F2, wherein the pathogenic infection is a C. difficile infection.
F4. The method of any one of Embodiments F1.1-F3, wherein the pathogenic infection is a recurrent C. difficile infection.
F5. The method of any one of Embodiments F1-F4, wherein the composition or food product is administered as one dose.
F6. The method of any one of Embodiments F1-F4, wherein the composition or food product is administered as multiple doses.
F7. The method of Embodiment F5 or F6, wherein each dose of the composition comprises administration of multiple capsules.
F8. The method of any one of Embodiments F1-F7, wherein the subject is administered an antibiotic prior to administration of the composition.
F9. The method of Embodiment F8, wherein the antibiotic is selected from the group consisting of vancomycin, kanamycin, gentamicin, colistin, metronidazole, clindamycin, fidaxomicin, penicillin, streptomycin, and cefoperazone.
F10. The method of Embodiment F8 or F9, wherein the antibiotic is vancomycin.
F11. The method of any one of Embodiments F1-F10, wherein the composition induces the proliferation and/or accumulation of regulatory T (Treg) cells in the subject.
F12. The method of any one of Embodiments F1-F11, wherein the subject is a human.

### EQUIVALENTS AND SCOPE

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in some embodiments, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in some embodiments, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. Each possibility represents a separate embodiment of the present invention.

It should be understood that, unless clearly indicated to the contrary, the disclosure of numerical values and ranges of numerical values in the specification includes both i) the exact value(s) or range specified, and ii) values that are “about” the value(s) or ranges specified (e.g., values or ranges falling within a reasonable range (e.g., about 10% similar)) as would be understood by a person of ordinary skill in the art.

It should also be understood that, unless clearly indicated to the contrary, in any methods disclosed herein that include more than one step or act, the order of the steps or acts of the method is not necessarily limited to the order in which the steps or acts of the method are disclosed.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United

States Patent Office Manual of Patent Examining Procedures, Section 2111.03.

### EXAMPLES

### Example 1: Pharmacodynamic Response to a Defined Bacterial Consortium in Patients with Clostridium difficile Infection (CDI): Results of a Phase 2 Study

### Background

Antibiotics used to treat Clostridium difficile infection (CDI) perturb the gut microbiome, increasing susceptibility to recurrent CDI (rCDI). Fecal microbiota transplants and donor-derived treatments promote the establishment of a gut environment resistant to CDI but present several challenges: (1) the composition and quality attributes of these are inherently variable; (2) they are difficult to scale up; and (3) they have resulted in transfer of harmful pathogens. In contrast, VE303 is a rationally defined consortium manufactured using clonal bacterial strains, obviating the need for donors and overcoming the limitations of donor-derived treatments. In the Phase 2 CONSORTIUM Study, the VE303 high dose arm (1.1×10¹¹ total CFU) had an acceptable safety profile and significantly reduced the risk of rCDI as compared with placebo. The high dose also induced superior VE303 strain colonization at 14 days, achieved long-term engraftment, and promoted early restoration of the microbiota and beneficial metabolites.

### Methods

The CONSORTIUM Study was a randomized, double-blind, placebo-controlled, dose-finding study in individuals at high risk of rCDI. After completing a course of antibiotics for a lab-confirmed CDI episode, subjects were randomized 1:1:1 to (1) low-dose VE303 (1.6×10⁸ CFU/day for 14 days; 2.2×10¹⁰ total CFU), (2) high-dose VE303 (8×10⁸ CFU/day for 14 days; 1.1×10¹¹ total CFU), or (3) placebo, dosed orally once daily for 14 consecutive days. Subjects were monitored for 24 weeks to evaluate safety, rCDI episodes, and gut microbiota composition. Fecal samples were collected during dosing, and at weeks 4 and 8 of monitoring. Metagenomic sequencing and metabolomics analysis were performed to identify associations between VE303 strain engraftment, resident microbes, stool metabolites, and clinical safety and efficacy.

### Results

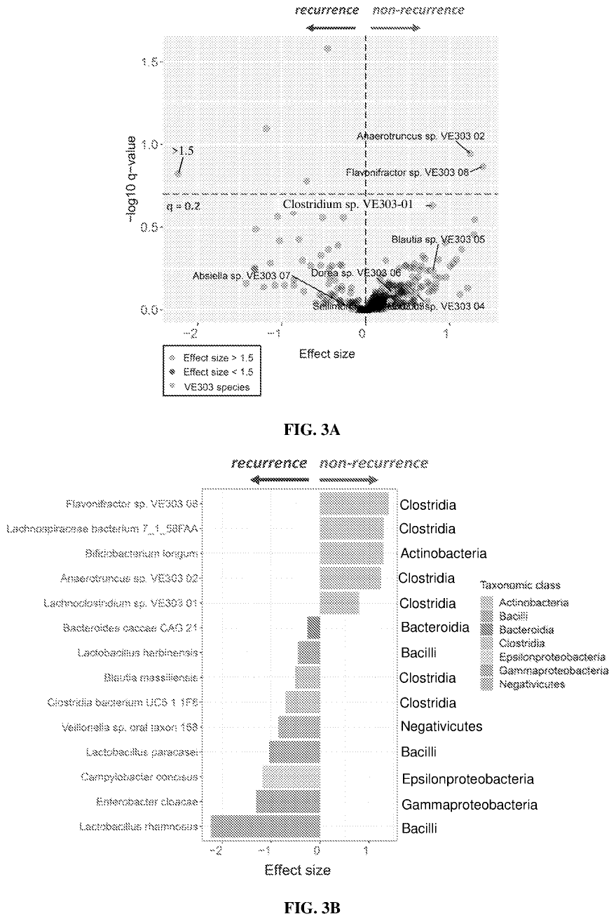

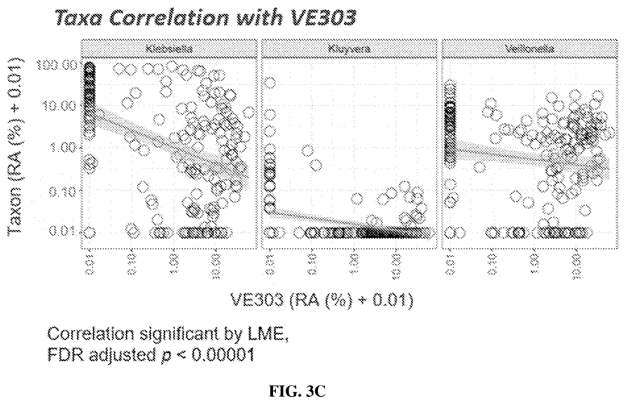

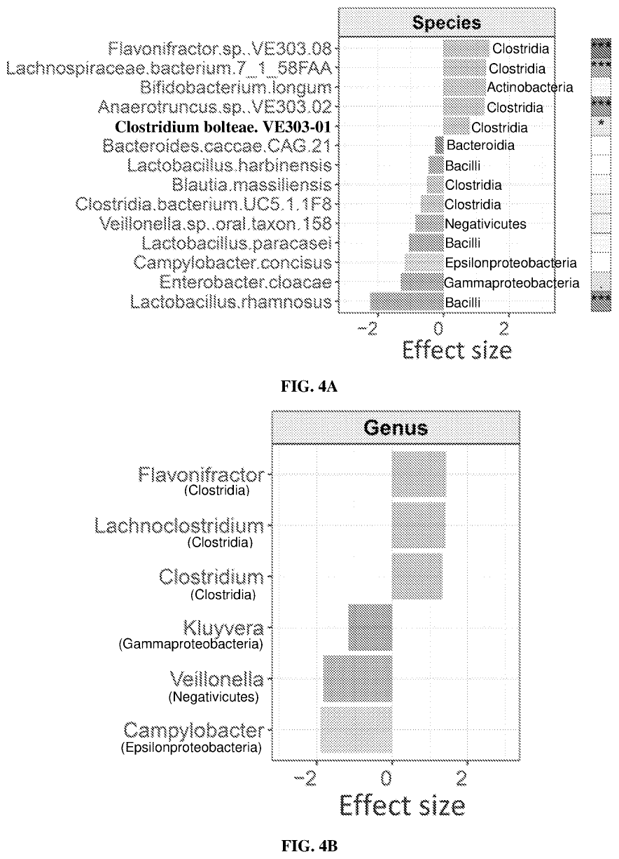

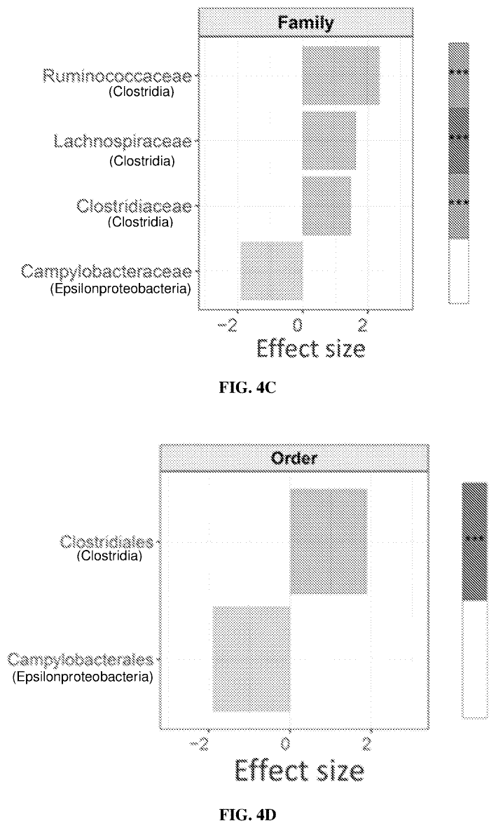

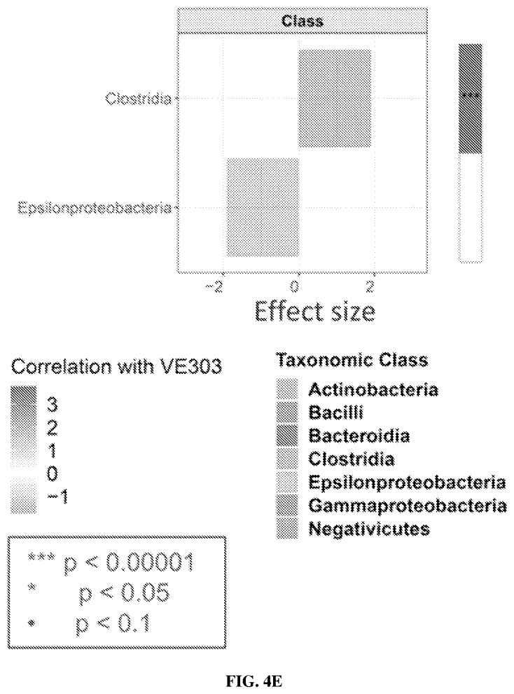

VE303-08 (Flavonifractor plautii), VE303-02 (Anaerotruncus colihominis), and VE303-01 (Clostridium bolteae) were to be significantly associated with nonrecurrence (see, FIGS. 3A, 3B, and 4A-4E, p-adjust<0.25, linear mixed effects [LME] model). Table E1-1 shows taxa associated with clinical response and VE303 at species and higher taxonomic levels, with greater Clostridia in nonrecurrent subjects and Proteobacteria in recurrent subjects. VE303 was negatively correlated (p-adjust<0.05, LME) with recurrence-associated taxa Veillonella and Kluyvera, and positively correlated with response-associated taxa.

TABLE E1-1
Bacterial Associations with Treatment Response (Recurrence or
Nonrecurrence at 8 weeks)

| | Associated with Nonrecurrence | Associated with Recurrence |
|---|---|---|
| | VE303 correlation: | VE303 correlation: |
| Taxa | positive (bolded below) | negative (bolded below) |
| Species | Anaerotruncus | Lactobacillus harbinensis |
| | colihominis(VE303-02) | |
| | Flavonifractor plautii | Campylobacter concisus |
| | (VE303-08) | |
| | Clostridium bolteae(VE303-01) | Lactobacillus rhamnosus |
| | Lachnospiraceae bacterium | Clostridia bacterium UC5.1.1F8 |
| | 7_1_58FAA | |
| | Bifidobacterium longum | Veillonella sp. oral taxon 158 |
| | | Lactobacillus paracasei |
| | | Blautia massiliensis |
| | | Bacteroides caccae CAG.21 |
| | | Enterobacter cloacae |
| Genus | Flavonifractor | Campylobacter |
| | Clostridium | Kluyvera |
| | Clostridium | Veillonella |
| Family | Ruminococcaceae | Campylobacteraceae |
| | Lachnospiraceae | |
| | Clostridiaceae | |
| Order | Clostridiales | Campylobacterales |
| Class | Clostridia | Epsilonproteobacteria |

Additionally, the relative abundances (as a proportion of a subject's total gut microbiota) of pathogenic taxa (Klebsiella, Kluyvera, and Veillonella) were negatively correlated with the relative abundance of VE303 strains (FIG. 3C). These results suggest that administration of VE303 suppresses putative pathogens, including Klebsiella, Kluyvera, Enterobacter, and Veillonella microorganisms, which are associated with C. difficile recurrence. Furthermore, multiple VE303 strains were positively correlated with gut metabolites including the short-chain fatty acids (SCFAs) valerate, acetate and butyrate, and the 2° bile acid ursodeoxycholic acid (UDCA) (see, FIG. 7, p-adjust<0.05, LME), which are thought to confer colonization resistance against C. difficile and inhibits spore germination.

### Conclusion

In subjects at high risk of rCDI, bacterial strains of VE303 were positively correlated with colonization by beneficial Clostridia strains, production of short-chain fatty acids, and production of UDCA, and negatively correlated with recurrence-associated Proteobacteria. VE303 may protect against rCDI through a combination of (i) direct exposure to the consortium strains and (ii) VE303-mediated recovery of the endogenous microbiota and metabolites.

### Example 2: Evaluation of Defined Bacterial Consortia in In Vivo Models of C. difficile Infection

### Mouse Husbandry

Experiments are performed using C57/BL6J mice purchased from The Jackson Laboratory (Bar Harbor, ME) and housed in ventilated sterile cages. All animals are maintained in a specific-pathogen-free facility. Animals are acclimated to the vivarium for at least 3 days prior to study (i.e., commencing antibiotic courses). For experiments involving C. difficile infection, mice are administered 10-10⁴ C. difficile strain VPI 10463 spores in 200 μL PBS by oral gavage. Experiments are performed in compliance with institutional guidelines and approved by the institution's Institutional Animal Care and Use Committee. Sterile food and drinking water are provided to the animals.

### Live Bacterial Product (LBP) Preparation

Individual bacterial strains are struck out from 15% glycerol freezer stocks onto EG (Eggerth Gagnon) agar plates containing 5% horse blood in an anaerobic chamber and incubated for 24-48 hours at 37° C. Colonies are inoculated into pre-reduced liquid Peptone Yeast Glucose (PYG) media and grown for 24-48 hours until dense (static in the anaerobic chamber). Optical density (OD₆₀₀) of the cultures is assessed and live bacterial product (LBP) cocktails are prepared inside an anaerobic chamber adjusting inputs based upon OD₆₀₀ for equal CFU ratio cocktails in PBS (sterile, pre-treated).

TABLE E2-1
LBP Compositions

| | | 16S rDNA |
|---|---|---|
| | | sequences |
| Composition | Species | (SEQ ID NO) |
| A | Clostridium bolteae | 1 |
| | Anaerotruncus colihominis | 2 |
| B | Clostridium bolteae | 1 |
| | Flavonifractor plautii | 8 |
| C | Anaerotruncus colihominis | 2 |
| | Flavonifractor plautii | 8 |
| D | Clostridium bolteae | 1 |
| | Anaerotruncus colihominis | 2 |
| | Flavonifractor plautii | 8 |
| E | Clostridium bolteae | 1 |
| | Anaerotruncus colihominis | 2 |
| | Sellimonas inteestinalis | 3 |
| | Clostridium symbiosum | 4 |
| | Blautia producta | 5 |
| | Dorea longicatena | 6 |
| | Clostridium innocuum | 7 |
| | Flavonifractor plautii | 8 |
| | Lachnospiraceae bacterium | 9 |
| F | Clostridium bolteae | 1 |
| | Anaerotruncus colihominis | 2 |
| | Sellimonas intestinalis | 3 |
| | Clostridium symbiosum | 4 |
| | Blautia producta | 5 |
| | Dorea longicatena | 6 |
| | Clostridium innocuum | 7 |
| | Flavonifractor plautii | 8 |
| | Bifidobacterium longum | 10 |
| G | Clostridium bolteae | 1 |
| | Anaerotruncus colihominis | 2 |
| | Sellimonas intestinalis | 3 |
| | Clostridium symbiosum | 4 |
| | Blautia producta | 5 |
| | Dorea longicatena | 6 |
| | Clostridium innocuum | 7 |
| | Flavonifractor plautii | 8 |
| | Lachnospiraceae bacterium | 9 |
| | Bifidobacterium longum | 10 |
| VE303 (positive control) | Clostridium bolteae | 1 |
| | Anaerotruncus colihominis | 2 |
| | Sellimonas intestinalis | 3 |
| | Clostridium symbiosum | 4 |
| | Blautia producta | 5 |
| | Dorea longicatena | 6 |
| | Clostridium innocuum | 7 |
| | Flavonifractor plautii | 8 |
| PBS (negative control) | — | — |

C. difficile Colony Forming Unit (CFU) Determination

Fecal pellets are collected, transported to an anaerobic chamber (<2 hours) and manually homogenized in 500 μL of pre-reduced PBS using a pipette tip and through repeated pipetting. Serial dilutions of fecal homogenates are prepared in pre-reduced PBS, 100 μL of which is spread onto cycloserine-cefoxitin-fructose agar with sodium taurocholate (TCCFA) plates and incubated anaerobically at 37° C. C. difficile CFUs are enumerated at 48 hours.

C. difficile Challenge Following LBP Colonization

Mice are treated with cefoperazone or another antibiotic, then administered one of a panel of Compositions provided in Table E2-1. Mice are then administered 10⁴ C. difficile spores by oral gavage. Following C. difficile challenge, mice are weighed regularly (e.g., daily) to assess morbidity as measured by weight loss, and mortality as measured by death following challenge. Mice that lose more than 20% body weight relative to baseline (before LBP administration, or before C. difficile challenge) are euthanized, and included in total mortality numbers. C. difficile burden is assessed by measuring CFUs in fecal pellets collected post-challenge (e.g., days 1, 3, 7, 14, and/or 21 post-inoculation with C. difficile).

### Induction of Treg Cells

A panel of Compositions provided in Table E2-1 is inoculated into separate germ-free mice (n=4 mice per Composition) by oral gavage. Mice are euthanized four weeks post-inoculation. Lamina propria leukocytes are isolated from colonic tissue of individual mice by standard methods and stained for analysis of cellular phenotypes by flow cytometry. Representative T cell surface markers include CD3, CD4, and CD8. Representative intracellular markers associated with Treg phenotypes include FoxP3, Helios, and RORY. Surface markers associated with Treg phenotypes including CD25, CD27, CD70, CD39, CD73, CTLA-4, GITR, LAG-3, LRRC32, and neuropilin-1. The abundance of Treg cells is measured by the abundance of CD4+FoxP3+ cells.

### Example 3: Pharmacodynamics of a Defined Bacterial Consortium in a Double-Blind Placebo-Controlled Phase 2 Study for Prevention of Recurrent C. difficile Infection

### 1. Summary

Clostridioides difficile infection (CDI) is typically treated with antibiotics, which perturbs the gut microbiome, thereby increasing susceptibility to recurrent CDI (rCDI). For patients who have experienced CDI recurrences, fecal microbiota transplant (FMT) and other donor-derived products have been successful in preventing further recurrences of CDI. The composition and quality attributes of these donor-derived procedures are inherently variable, they have occasionally resulted in transmission of harmful pathogens from the human donors of the feces (DeFilipp et al., 2019), the procedure requires extensive and costly donor screening (Craven et al., 2017), and the precise components of the gastrointestinal microbiota that provide resistance against C. difficile remain undetermined (Khanna et al., 2021; McDonald et al., 2018). VE303 is a rationally defined consortium developed for the prevention of recurrent CDI that overcomes the limitations of donor-derived approaches by virtue of being produced by fermentation from clonal cell banks, obviating the need for donors. In the Phase 2 clinical study, VE303-002, for the prevention of recurrent CDI, subjects given the high dose of VE303 had a significantly improved clinical response compared with placebo recipients.

This Example uses a previously published framework (Dsouza et al., 2022) to quantify the pharmacokinetics (PK) and pharmacodynamics (PD) of VE303 in the stool of subjects at risk of recurrent CDI. Results indicate that both the VE303 community and individual VE303 strains rapidly colonized the gut environment of subjects with CDI in a dose-dependent manner, with the greatest colonization observed in the high-dose VE303 group. Increased VE303 colonization at the end of dosing was associated with a lower probability of CDI recurrence regardless of the VE303 dose received, establishing a direct connection between exposure and clinical response. Subjects enrolled in the Phase 2 study who received vancomycin four times per day (QID) for their qualifying CDI episode showed significantly greater depletion of host microbiota diversity and superior colonization of VE303 in comparison with those receiving fidaxomicin.

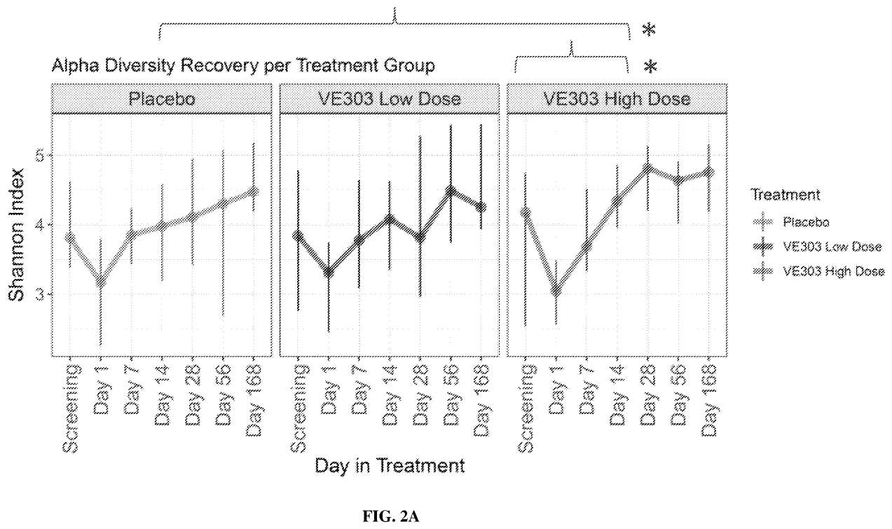

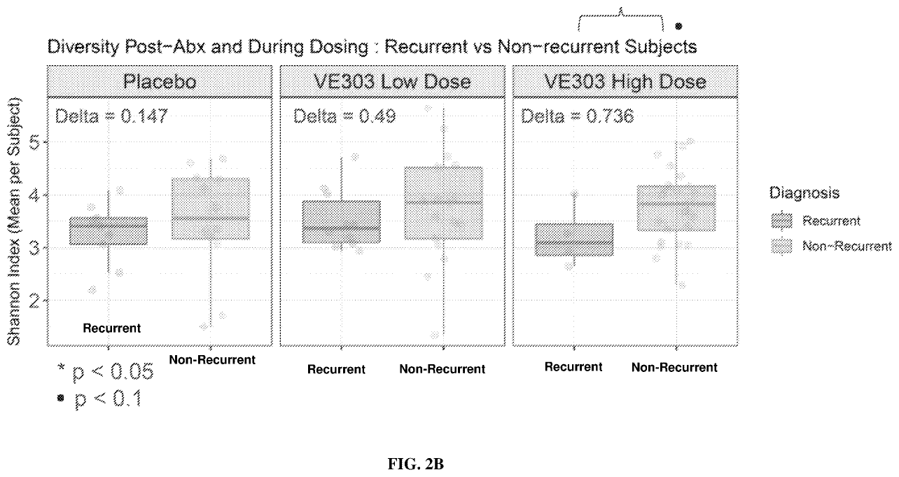

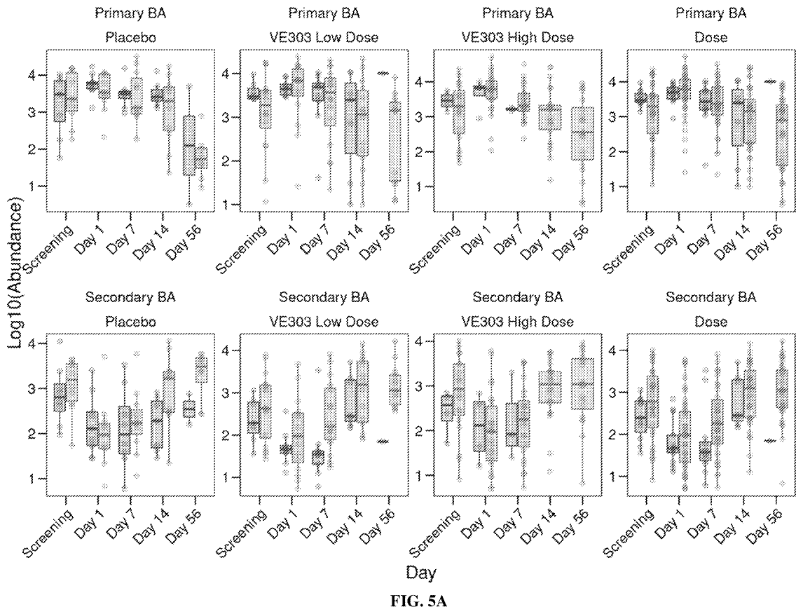

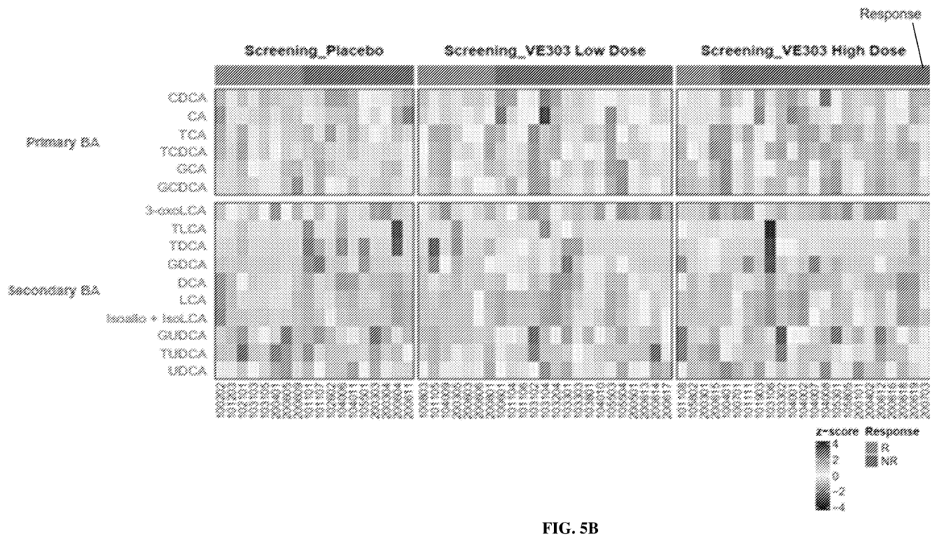

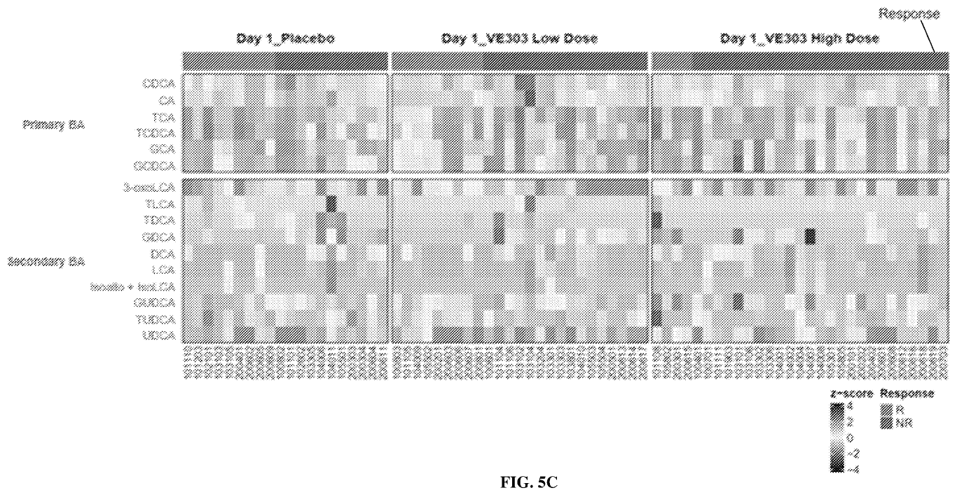

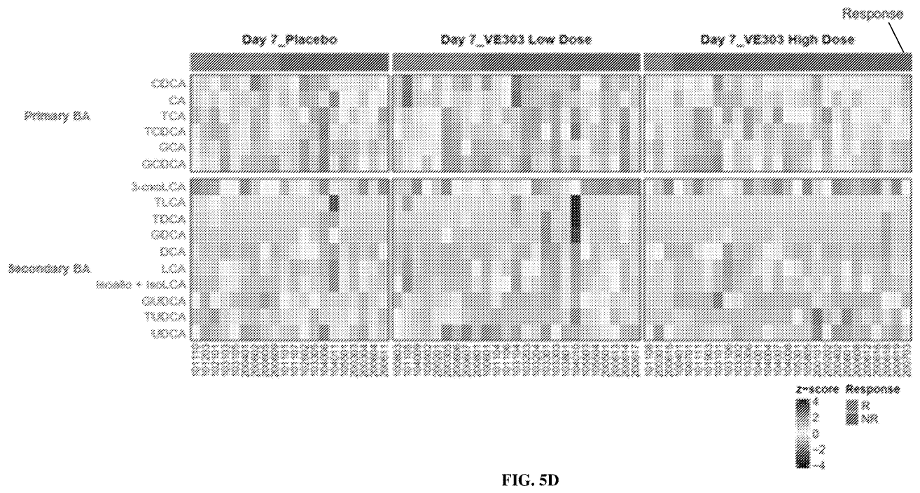

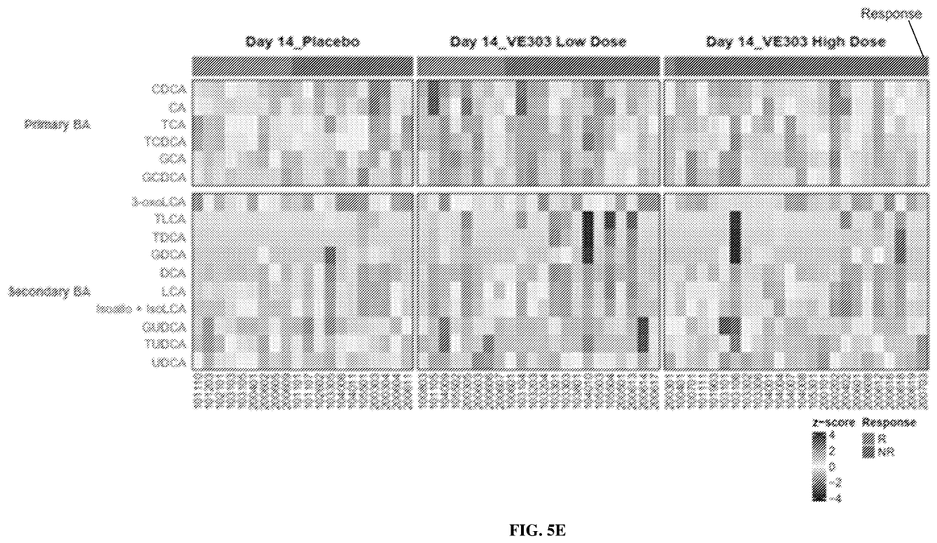

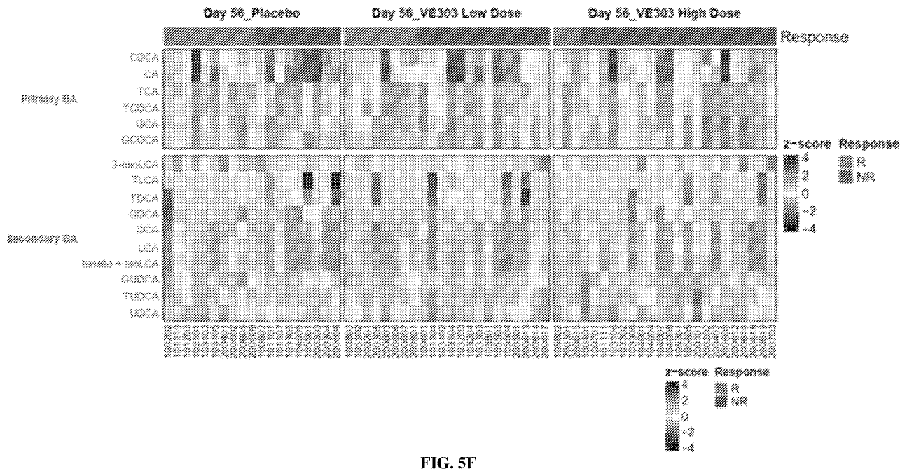

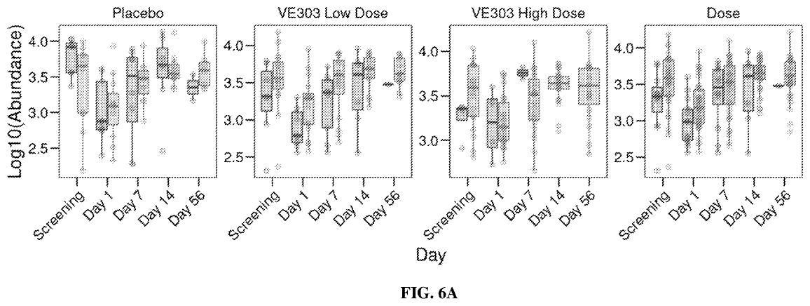

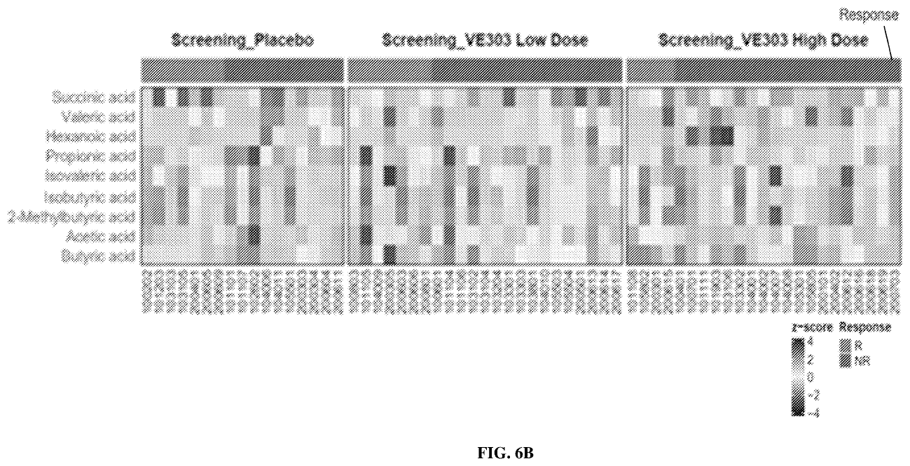

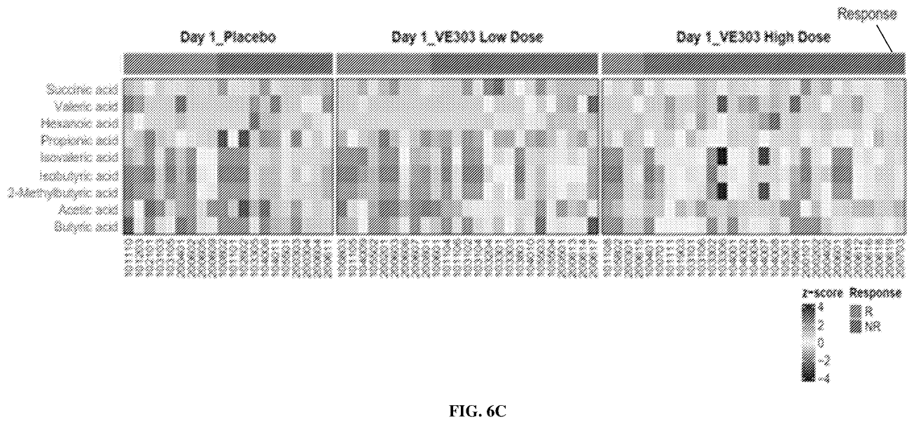

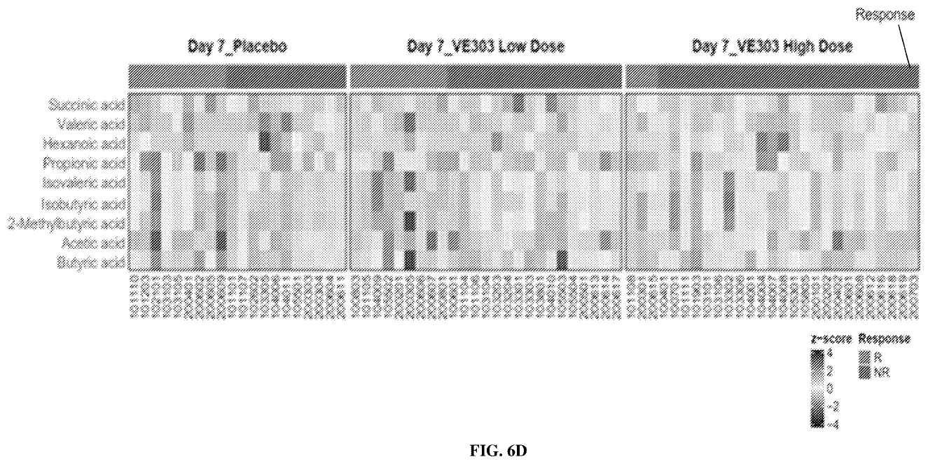

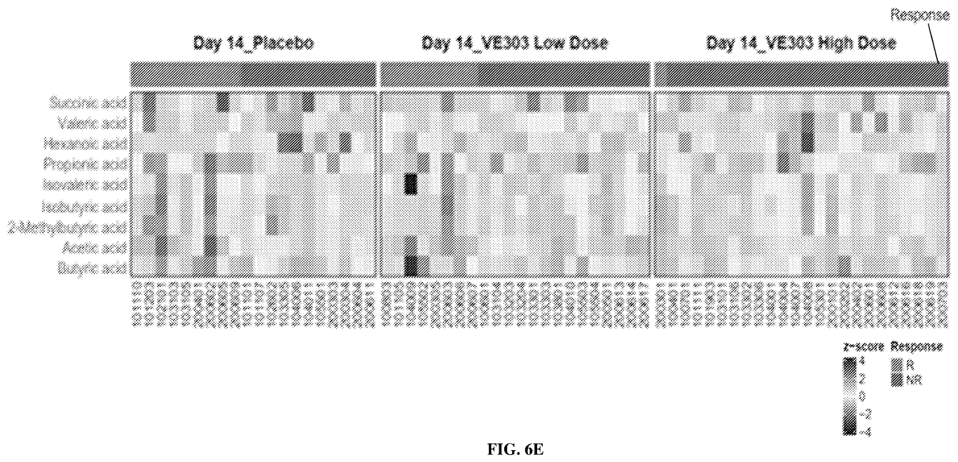

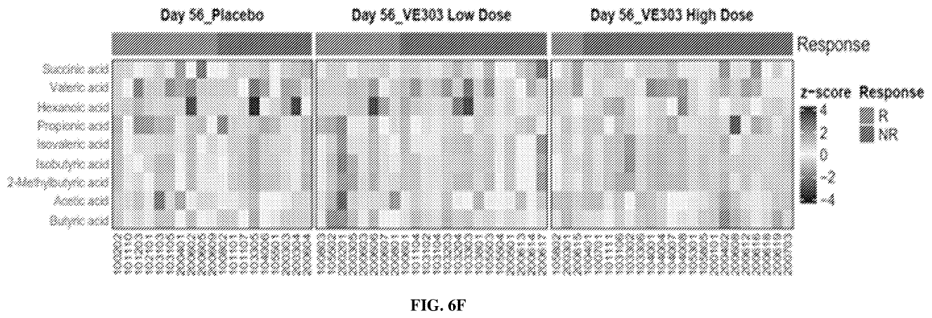

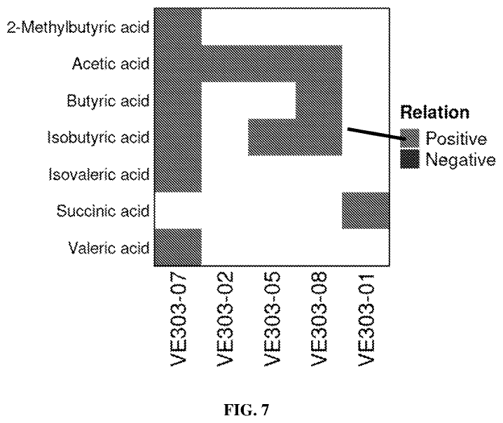

Treatment with VE303 facilitated PD recovery of the microbial community and of beneficial metabolites in these subjects. Species diversity recovered rapidly, particularly in the high-dose VE303 group, and was associated with non-recurrence of CDI (FIGS. 2A-2B). VE303 organisms were negatively correlated with recurrence-associated pathogens and positively correlated with response-associated commensals (FIGS. 3A-4E), short-chain fatty acids (SCFAs) (FIGS. 6A-6F), and the secondary bile acid ursodeoxycholic acid (UDCA) (FIGS. 5A-5F). In summary, the results of Study VE303-002 demonstrate that higher VE303 colonization and VE303-mediated recovery of the endogenous microbiota and metabolites contributed to the improved efficacy observed in the high-dose VE303 treatment arm (FIGS. 2A, 2B, and 5A-7).

### 2. Methods

Stool samples were collected longitudinally from each subject in Study VE303-002, at Screening and on Study Days 1, 7, 14, 28, 56, and 168. These samples were used to determine the prevalence and abundance of the eight VE303 consortium bacteria during and after treatment with VE303, to characterize the endogenous bacterial community, and to quantify stool metabolite concentrations.

### 2.1 Sample Processing and Stool Metagenomic Sequencing

Stool samples were collected fresh and approximately 500 mg was transferred to an OMNIgene-GUT (OMR-200) tube and resuspended in the preservation buffer according to the manufacturer's instructions. Tubes were shipped to Diversigen, a microbiome analytics laboratory (Minneapolis, Minnesota, United States of America [USA]), where preserved stool suspensions were extracted and sequenced on the Illumina NovaSeq platform using standard operating procedures. Stool metagenomic sequences (or reads) were assigned to taxonomy using the k-mer-based One Codex platform (onecodex.com).

### 2.2 Analytical Methods for VE303 Strain Detection and Community Characterization

VE303 strains were detected in subject stool samples using a bioinformatics assay previously described (Dsouza et al., 2022). Briefly, a set of unique genomic markers spanning the entire length of the strain genome were identified for each of the VE303 organisms. The presence or absence of each VE303 strain in each subject stool sample was determined by the depth and coverage of the unique genomic markers in the metagenomic reads. Detection precision and sensitivity of the method was determined using human stool that was spiked with varying amounts of evenly mixed VE303 strain deoxyribonucleic acid (DNA) (10%, 1%, and 0.1% total community abundance) before metagenomic sequencing; individual strains were recovered at expected relative abundance (RA) in stool spike-ins, with a limit of detection of 0.0125% abundance per strain. No VE303 strains were detected in the human fecal DNA that did not contain the VE303 spike-in DNA (Dsouza et al., 2022). The relative abundance of individual VE303 strains was calculated as the total marker depth per strain (the ratio of metagenomic reads matching any strain marker to the total number of strain markers) divided by the total bases and multiplied by the genome size. RA=(total marker depth/total bases per sample)*genome size.

The estimated relative abundance of endogenous bacterial species in the microbial community was determined using the standard One Codex algorithm from quality-filtered metagenomic reads after removal of unclassified reads and reads that map to the human host (Minot et al. 2015) (onecodex.com). At higher taxonomic levels (e.g., Phylum or Class level), the relative abundance was calculated for each sample as the ratio of sequence reads assigned at the desired taxonomic level (plus all reads assigned below) to the total number of assigned reads.

### 2.3 Bile Acid and Short-Chain Fatty Acid Quantification

Stool samples were collected longitudinally, and the concentration of bile acids (BA) and SCFA was determined as previously described (Dsouza et al., 2022). The following BAs were measured by high-performance liquid chromatography (HPLC) and compared with internal standards (Metabolon Incorporated, Durham, North Carolina, USA):

chenodeoxycholic acid (CDCA), cholic acid (CA), taurocholic acid (TCA), taurochenodeoxycholic acid (TCDCA), glycocholic acid (GCA), glycochenodeoxycholic acid (GCDCA), dehydrolithocholic acid (herein 3-oxoLCA), deoxycholic acid (DCA), lithocholic acid (LCA), ursodeoxycholic acid (UDCA), glycodeoxycholic acid (GDCA), glycoursodeoxycholic acid (GUDCA), taurodeoxycholic acid (TDCA), taurolithocholic acid (TLCA), and tauroursodeoxycholic acid (TUDCA). Isoallo- and isolithocholic acid were also measured, but their peak areas were indistinguishable from each other in the assay. These data were reported as “isoallo-+isoLCA”. Approximately 8 to 12 mg of lyophilized human stool was used for each sample. The peak area of each BA was measured against the internal standards and corrected for sample weight (dry lyophilized stool). The final concentrations were provided as ng BA/mg lyophilized feces.

Stool SCFA concentrations, including acetic acid, propionic acid, isobutyric acid, butyric acid, 2-methylbutyric acid, isovaleric acid, valeric acid, succinic acid, and hexanoic acid were determined by liquid chromatography with tandem mass spectrometry (LC-MS/MS) and compared with internal standards (Metabolon, Incorporated, Durham, North Carolina, USA). Approximately 100 mg of raw, frozen human stool was used for each sample. The peak area of each SCFA was measured against the internal standards and corrected for sample weight (wet stool). The final concentrations were provided as ng SCFA/mg feces.

### 2.4 Models to Determine Associations Between Endogenous Taxa, Metabolites, Response, and VE303

The species alpha diversity, abundance of endogenous microbiota, SCFA, and BA were compared across treatment groups and by recurrence status using an LME model (Dsouza et al., 2022; Mallick et al., 2021). The model was also used to assess the associations of the microbiota and metabolite concentrations with total VE303 abundance during the early recovery (t≤Day 14) time period (Section 3. Pharmacodynamics). Treatment group, standard-of-care (SoC) antibiotic type (vancomycin, fidaxomicin, or metronidazole), recurrence status, and sequencing batch were included as categorical covariates in the model, with VE303 abundance included as a continuous covariate; repeated sampling per subject was handled as a random effect following the equation: Diversity/Metabolite/Microbial Taxon ~ Treatment (high-dose (HD)/low-dose (LD)/Placebo)+Abx Type+Recurrence+Batch+VE303+1|Subject. The diversity differences between treatment groups per timepoint, diversity change from Screening to Day 14, and delta diversity (recurrent-non-recurrent) per treatment group were assessed by fitting additional models that encoded an interaction between treatment and Visit, or treatment and recurrence status. The model was fit using early dosing timepoints (t≤day 14), and excluding all samples collected after the onset of a CDI recurrence event or after the onset of on-study antibiotics, whichever occurred first.

### 3 Pharmacodynamics

Patients with CDI can display gut dysbiosis characterized by low species diversity and dominance of putative pathogens, which is further exacerbated by first-line treatment with antibiotics. Gut perturbation with antibiotic treatment can also lead to an imbalance in bacterial taxa in the gut, namely a low abundance of commensal organisms from the bacterial classes Clostridia and Bacteroidia and a high abundance of Gammaproteobacteria. This microbiota imbalance (or dysbiosis) is accompanied by a reduction of beneficial bacterial metabolites such as SCFAs and secondary BAs, and an over-abundance of primary BAs. Gut microbe-produced SCFAs enhance colonization resistance against C. difficile and protect against C. difficile-induced colitis (Fachi et al., 2019; Theriot et al., 2014). Similarly, secondary BAs are thought to play at role in establishing colonization resistance against CDI. The abundance of primary BAs after antibiotic treatment in patients with CDI can trigger C. difficile spore germination and permit outgrowth of vegetative cells (Sorg and Sonenshein, 2008; Foley et al., 2019; Seekatz et al., 2018; Winston and Theriot, 2019). However, recovery of the secondary BA pool abates spore germination and growth (Foley et al., 2019; Kang et al., 2019; Palmieri et al., 2018; Seekatz et al., 2018; Thanissery et al., 2017). As described herein, the effect of VE303 dosing on the recovery of the endogenous microbiota and metabolites was examined to determine the PD effects of VE303. The effect of VE303 administration on species alpha diversity, the resident bacterial community, and stool SCFA and BA concentrations was determined. Regression analysis was also conducted to establish correlative links between the gut microbiome and metabolites with CDI non-recurrence.

Species alpha diversity was calculated using the Shannon Index as a measure of early microbiome recovery (up to Day 14) over time across treatment groups and clinical response categories. Species diversity recovered over time after antibiotic treatment and was significantly associated with non-recurrence for all treatment groups (p=0.02) in the LME model (FIGS. 2A-2B, Table E3-1). However, species recovery was most pronounced in the VE303 HD group; the delta (relative diversity change) in non-recurrent versus recurrent subjects increased with dose and was highest for the VE303 HD group (Placebo: Delta=0.14, p=0.29, Low Dose: Delta=0.49, p=0.39, High Dose: Delta=0.73, p=0.08; LME, Table E3-1); diversity recovered to greater than screening levels by Day 14 in the VE303 HD group (p=0.004, LME); in contrast, the difference was not significant in VE303 LD and placebo groups (p>0.2, LME). Also, Day 14 diversity was increased in the VE303 HD group versus placebo (p=0.02, LME), whereas the VE303 LD group was not significantly different from the placebo group. Collectively, these data suggest that VE303-mediated expedited recovery of the endogenous microbiota contributed to the improved efficacy observed in the VE303 HD arm.

TABLE E3-1
Linear Mixed Model Summary: Comparison of Diversity Across
and Response Groups over Time

| Contrasts | Covariate | Effect Size | Std. Error | p-value |
|---|---|---|---|---|
| Treatment | VE303 low-dose(LD) vs Placebo | 0.076 | 0.221 | 0.731 |
| | VE303 high-dose(HD) vs Placebo | 0.266 | 0.213 | 0.218 |
| On-study Response | non-Recurrent vs Recurrent | 0.443 | 0.195 | 0.025 |
| Treatment: Response | Placebo: non-Recurrent vs | 0.337 | 0.321 | 0.299 |
| | Recurrent | | | |
| | VE303 LD: non-Recurrent vs | 0.272 | 0.316 | 0.391 |
| | Recurrent | | | |
| | VE303 HD: non-Recurrent vs | 0.810 | 0.457 | 0.079 |
| | Recurrent | | | |
| Treatment: Timepoint | Placebo: Day 14 vs Screening | −0.204 | 0.245 | 0.406 |
| | VE303 LD: Day 14 vs Screening | 0.175 | 0.226 | 0.440 |
| | VE303 HD: Day 14 vs Screening | 0.654 | 0.226 | 0.004 |
| Timepoint: Treatment | Day 14: VE303 LD vs Placebo | 0.277 | 0.312 | 0.376 |
| | Day 14: VE303 HD vs Placebo | 0.705 | 0.306 | 0.022 |

Models were fit to examine the differences in diversity due to a) Treatment group: VE303-dosed versus placebo subjects across all timepoints (Contrasts=Treatment) at Day 14 only (Contrasts=Timepoint: Treatment). b) On-study Response: non-Recurrent versus recurrent subjects regardless of treatment group (Contrasts=Response) and per treatment group (Contrasts=Treatment: Response). c) Time: Day 14 versus Screening per treatment group (Contrasts=Treatment: Timepoint). A positive Effect Size shows an increased diversity in Dosed (compared with placebo)/non-Recurrent (compared to recurrent)/Day 14 (compared with Screening) groups.

Metagenomics sequencing and analysis of stool samples from subjects was conducted to identify endogenous bacterial taxa associated with recurrence and non-recurrence of CDI. Taxonomic abundance for each organism was calculated using the bioinformatics methods described in Section 2.1. Statistical analysis to identify bacterial associations with recurrence and VE303 abundance was conducted using the LME model described in Section 2. Methods. The analysis focused on samples collected up to Day 14, during the period when subjects were thought to be at higher risk for CDI recurrence. Analysis was conducted at the species, genus, family, order, and class taxonomic levels. Prevalence and abundance thresholds were applied to exclude taxa that were too sparse or low in abundance to obtain accurate model results (prevalence threshold=present in at least 20% of samples, abundance threshold=0.01% average abundance across subjects).

FIG. 3A shows the associations between species abundance and CDI recurrence through Day 56 in the VE303-dosed groups for 378 bacterial species included in the analysis. Most species associations were not statistically significant, with a false discovery rate (FDR) adjusted p-value >0.2, potentially due to the reduced number of samples (only Day 1, 7, and 14 were included) and the unbalanced nature of the dataset (n=14 with recurrence, n=42 without recurrence). VE303 organisms (species VE303-08 and VE303-02) were found to be significantly associated with non-recurrence (FDR adjusted p-value <0.2), whereas Enterobacter cloacae and Lactobacillus rhamnosus were significantly associated with recurrence from among all analyzed species (FIGS. 3A-3B). Species associated with non-recurrence belonged to class Clostridia or Actinobacteria; those associated with recurrence included Negativicutes, Proteobacteria, and Bacilli (FIG. 3B). Similar patterns were seen at higher taxonomic levels (FIGS. 4A-4E), with a preponderance of Clostridia in non-recurrent subjects and Proteobacteria in recurrent subjects. These observations are consistent with prior work showing that the presence and recovery of commensal microbial organisms such as Clostridia, Bifidobacterium and Faecalibacterium spp. are associated with protection against recurrence of CDI (Khanna et al., 2016; Martinez et al., 2022; Yun et al., 2017), whereas resurgence of potential pathogens such as Enterobacteriaceae, Veillonella, and Klebsiella can be linked to CDI recurrence and exacerbation of antibiotic-associated diarrhea (Khanna et al., 2016; Larcombe et al., 2018; Motamedi et al., 2021; Zollner-Schwetz et al., 2008). Importantly, VE303 was significantly negatively correlated (FDR adjusted p-value <0.05) with recurrence-associated taxa Veillonella and Kluyvera, and positively correlated with response-associated taxa (FIGS. 4A-4E). Together, these results suggest that, in addition to facilitating an expedited increase in gut microbial diversity, administration of VE303 may drive changes in abundance of specific bacteria to protect against CDI recurrence.

Stool concentrations of BAs and SCFAs were measured over time to determine the effect of VE303 treatment on the recovery of metabolites in the gut of enrolled subjects. Stool samples were collected at Screening, Day 1, Day 7, Day 14, and Day 56 study timepoints and subjected to metabolomic analysis (Section 2. Methods). Consistent with our observations in Study VE303-01 (Dsouza et al., 2022), the secondary BAs decreased following antibiotic administration, from a median concentration of 540.7 ng/mg in all VE303-dosed subjects at Screening to a median of 59.4 ng/mg at Day 1 (FIG. 5A). A similar reduction in secondary BAs was observed in the placebo group, as the median concentration was reduced from 917.7 ng/mg at screening to 110.8 ng/mg at Day 1. The reduction in secondary BAs from screening to Day 1 corresponded with an increase in primary BAs in the stool for all subjects. Over time and after the start of VE303 or placebo administration, the total concentration of secondary BAs increased, consistent with the expected recovery of the gut metabolite pool (Brown et al., 2018; Seekatz et al., 2018; Weingarden et al., 2014). There were no significant differences in the rate of BA recovery in any of the treatment groups or between subjects with or without an on-study CDI recurrence. Likewise, there were no significant differences in the recovery of individual primary or secondary BAs (FIG. 5B-5F).

Antibiotic treatment for the qualifying CDI episode led to a reduction in the concentration of SCFAs in the stool for all subjects. In all VE303-dosed subjects, the total SCFA levels decreased from a median concentration of 3375.4 ng/mg at screening to a median of 1404.8 ng/mg at Day 1 (FIG. 6A). A similar reduction in SCFAs was observed in the placebo group, with the median concentration reduced from 4983.3 ng/mg at screening to 1103.9 ng/mg at Day 1. Like the secondary BA levels, over time and after the start of VE303 or placebo administration, the total concentration of SCFAs increased. There were no significant differences in the rate of SCFA recovery in any of the treatment groups or between subjects with or without an on-study CDI recurrence. Likewise, there were no significant differences in the recovery of individual SCFAs (FIGS. 6B-6F).

To determine whether BA and SCFA concentrations were associated with VE303 strain colonization through Study Day 14 in Study VE303-002, an LME model was used to correlate metabolite concentrations with VE303 strain relative abundance in VE303-dosed subjects. The analysis was focused on samples collected up to Day 14, during the period when subjects were thought to be at higher risk for CDI recurrence. The relative abundance of 5 of 8 VE303 strains (VE303-01, -02, -05, -07, and -08) was positively correlated with elevated levels of multiple SCFAs in the stool (FDR-adjusted p<0.05, LME, FIG. 7, Table E3-2). Notably, strains VE303-07 and VE303-08 were significantly associated with a higher butyrate concentration, which has been reported to involved in colonization resistance against C. difficile (Theriot et al., 2014) and protection against C. difficile-induced colitis (Fachi et al., 2019). The relative abundance of 3 of 8 VE303 strains (VE303-05, -07, and -08) was also positively correlated with the concentration of UDCA (FDR-adjusted p<0.05, Table E3-2), a secondary BA that has been linked to gut health and has direct inhibitory effects on C. difficile spore germination (Weingarden et al., 2016).

TABLE E3-2
Linear Mixed Model Summary: Correlation Between Short-Chain Fatty
Acids (SCFA), Bile Acids (BA) and VE303 Strain Abundance Up
to Day 14

| | | Effect | Std. | p- | adjusted |
|---|---|---|---|---|---|
| | Strain | Size | Error | value | p-value |
| SCFA | | | | | |
| 2-Methylbutyric acid | VE303.07 | 2.09 | 0.64 | 0.0047 | 0.015 |
| 2-Methylbutyric acid | VE303.05 | 1.18 | 0.50 | 0.0314 | 0.059 |
| 2-Methylbutyric acid | VE303.08 | 1.07 | 0.50 | 0.0470 | 0.081 |
| 2-Methylbutyric acid | VE303.02 | 1.02 | 0.52 | 0.0684 | 0.108 |
| Acetic acid | VE303.05 | 1.60 | 0.39 | 0.0008 | 0.003 |
| Acetic acid | VE303.08 | 1.47 | 0.39 | 0.0014 | 0.005 |
| Acetic acid | VE303.07 | 1.83 | 0.55 | 0.0041 | 0.013 |
| Acetic acid | VE303.02 | 1.16 | 0.45 | 0.0195 | 0.042 |
| Acetic acid | VE303.04 | 1.27 | 0.74 | 0.1047 | 0.156 |
| Butyric acid | VE303.07 | 2.99 | 0.73 | 0.0007 | 0.003 |
| Butyric acid | VE303.08 | 1.59 | 0.61 | 0.0187 | 0.041 |
| Butyric acid | VE303.05 | 1.45 | 0.6 | 0.0305 | 0.058 |
| Butyric acid | VE303.02 | 1.05 | 0.61 | 0.1040 | 0.156 |
| Hexanoic acid | VE303.07 | 0.93 | 0.50 | 0.0771 | 0.119 |
| Hexanoic acid | VE303.08 | 0.64 | 0.36 | 0.0924 | 0.140 |
| Hexanoic acid | VE303.05 | 0.59 | 0.37 | 0.1332 | 0.194 |
| Hexanoic acid | VE303.04 | 0.91 | 0.62 | 0.1568 | 0.223 |
| Hexanoic acid | VE303.02 | 0.56 | 0.39 | 0.1692 | 0.237 |
| Isobutyric acid | VE303.05 | 1.22 | 0.38 | 0.0051 | 0.016 |
| Isobutyric acid | VE303.07 | 1.41 | 0.56 | 0.0233 | 0.049 |
| Isobutyric acid | VE303.08 | 0.99 | 0.39 | 0.0236 | 0.049 |
| Isobutyric acid | VE303.02 | 0.92 | 0.42 | 0.0446 | 0.078 |
| Isovaleric acid | VE303.07 | 1.82 | 0.59 | 0.0068 | 0.020 |
| Isovaleric acid | VE303.08 | 1.00 | 0.45 | 0.0400 | 0.072 |
| Isovaleric acid | VE303.05 | 0.97 | 0.47 | 0.0554 | 0.093 |
| Isovaleric acid | VE303.04 | 1.21 | 0.79 | 0.1420 | 0.206 |
| Propionic acid | VE303.04 | 1.28 | 0.53 | 0.0278 | 0.055 |
| Propionic acid | VE303.06 | 1.32 | 0.88 | 0.1521 | 0.218 |
| Succinic acid | VE303.01 | 7.45 | 2.04 | 0.0020 | 0.007 |
| Succinic acid | VE303.05 | 0.88 | 0.55 | 0.1289 | 0.189 |
| Valeric acid | VE303.07 | 2.28 | 0.73 | 0.0059 | 0.018 |
| Valeric acid | VE303.08 | 1.21 | 0.56 | 0.0436 | 0.078 |
| Valeric acid | VE303.02 | 0.87 | 0.59 | 0.1577 | 0.223 |
| BA | | | | | |
| Alloiso_Isolithocholic | VE303.03 | −5.044 | 2.258 | 0.0392 | 0.103 |
| Acid | | | | | |
| Chenodeoxycholic Acid | VE303.08 | 1.426 | 0.813 | 0.0974 | 0.190 |
| Cholic Acid | VE303.03 | 3.259 | 1.959 | 0.1146 | 0.216 |
| Dehydrolithocholic | VE303.01 | 9.720 | 4.931 | 0.0652 | 0.143 |
| Acid | | | | | |
| Dehydrolithocholic | VE303.02 | 1.691 | 1.038 | 0.1218 | 0.224 |
| Acid | | | | | |
| Deoxycholic Acid | VE303.03 | −5.744 | 2.861 | 0.0608 | 0.137 |
| Deoxycholic Acid | VE303.01 | 9.374 | 4.979 | 0.0770 | 0.162 |
| Glycochenodeoxycholic | VE303.02 | −1.371 | 0.875 | 0.1357 | 0.239 |
| Acid | | | | | |
| Glycocholic Acid | VE303.02 | −1.881 | 0.937 | 0.0608 | 0.137 |
| Glycodeoxycholic Acid | VE303.03 | −1.515 | 0.979 | 0.1399 | 0.245 |
| Lithocholic Acid | VE303.03 | −5.148 | 2.676 | 0.0713 | 0.154 |
| Lithocholic Acid | VE303.01 | 7.201 | 4.684 | 0.1426 | 0.249 |
| Taurochenodeoxycholic | VE303.06 | 3.608 | 2.274 | 0.1310 | 0.235 |
| Acid | | | | | |
| Taurocholic Acid | VE303.06 | 4.782 | 2.539 | 0.0768 | 0.162 |
| Taurodeoxycholic Acid | VE303.08 | −0.675 | 0.355 | 0.0748 | 0.160 |
| Taurodeoxycholic Acid | VE303.03 | −1.436 | 0.800 | 0.0905 | 0.180 |
| Taurodeoxycholic Acid | VE303.01 | 2.250 | 1.426 | 0.1331 | 0.236 |
| Taurolithocholic Acid | VE303.03 | −1.199 | 0.688 | 0.0993 | 0.193 |
| Tauroursodeoxycholic | VE303.04 | 2.569 | 1.102 | 0.0324 | 0.092 |
| Acid | | | | | |
| Tauroursodeoxycholic | VE303.08 | 1.241 | 0.678 | 0.0849 | 0.172 |
| Acid | | | | | |
| Tauroursodeoxycholic | VE303.05 | 1.140 | 0.712 | 0.1278 | 0.231 |
| Acid | | | | | |
| Ursodeoxycholic Acid | VE303.08 | 3.174 | 0.976 | 0.0047 | 0.020 |
| Ursodeoxycholic Acid | VE303.07 | 4.290 | 1.362 | 0.0058 | 0.024 |
| Ursodeoxycholic Acid | VE303.05 | 3.051 | 1.043 | 0.0094 | 0.036 |
| Ursodeoxycholic Acid | VE303.04 | 3.411 | 1.819 | 0.0781 | 0.163 |

A linear mixed model was fit to correlate total VE303 strain abundance and concentrations of short-chain fatty acids (SCFA) and bile acids (BA) in VE303-dosed groups up to Day 14. A negative value for the Effect Size indicates negative correlation between the indicated metabolite and VE303 abundance. Associations with an adjusted p-value >0.25 are not shown.

The following conclusions can be drawn regarding the PD recovery of the resident microbiota and metabolites following VE303 or placebo administration in Study VE303-002:
The VE303 HD was associated with faster recovery of species diversity, which in turn was positively correlated with non-recurrence of CDI.
Species VE303-02 and VE303-08 were the top two species associated with non-recurrence from among 378 analyzed species in the VE303-dosed groups.
Through Study Day 14, VE303 abundance was negatively correlated with recurrence-associated Proteobacteria and positively correlated with response-associated commensal bacteria at multiple taxonomic levels, indicating a VE303-mediated beneficial shift in the host bacterial ecosystem.
Multiple VE303 strains were positively correlated with SCFAs (including acetate and butyrate) and the secondary BA UDCA, which is known to confer colonization resistance against C. difficile and inhibit spore germination.

### 4. Discussion

The PK and PD of administration of VE303 in Study VE303-002 was assessed. Metagenomics sequencing and metabolite datasets were analyzed to quantify VE303 exposure in subjects with CDI and to identify potential VE303 mechanisms of action. Study subpopulations, including stratifications by SoC antibiotic treatment regimen and number of previous CDI episodes, were also examined to understand their respective effects on host bacterial diversity and subsequent VE303 colonization.

To assess PD in the study subjects, species alpha diversity was calculated (using the Shannon Index) as a measure of early microbiome recovery across treatment and clinical response groups. The recovery of diversity from Day 1 to Day 14 and the relative diversity change in subjects with versus without an on-study CDI recurrence were greatest in the VE303 HD group; the difference was not statistically significant in VE303 LD and placebo groups. VE303 species had a strong association with prevention of an on-study CDI recurrence from among the 378 endogenous bacterial species identified in the VE303-dosed groups. Additionally, VE303 abundance was strongly negatively correlated with recurrence-associated Proteobacteria and positively correlated with beneficial Clostridia, indicating that recovery of the microbiota community likely contributes to the VE303 mechanism of action. The increase in diversity was accompanied by a recovery of secondary BAs and SCFAs, and a reduction in primary BAs over time for all study subjects. Furthermore, consistent with in-silico and in-vitro predictions (Dsouza et al., 2022), multiple VE303 organisms were positively correlated with SCFAs (notably acetate, butyrate, and valerate) and with the secondary BA UDCA, which is known to provide colonization resistance against C. difficile and inhibit spore germination.

The findings presented in this Example collectively indicate that greater exposure to VE303 strains individually and in total contributed to the improved efficacy observed in the VE303 HD arm of Study VE303-002. Administration of VE303 may protect against CDI recurrence through a combination of direct exposure to—and expansion of—the bacterial strains of VE303 and VE303-mediated recovery of the endogenous microbiota and metabolites. A Phase 3 study is planned to determine the efficacy of VE303 administered at the high dose to prevent CDI recurrence in a larger cohort of subjects.

### 5. References

Britton, R. A., and Young, V. B. (2014). Role of the intestinal microbiota in resistance to colonization by Clostridium difficile. Gastroenterology 146, 1547-1553. https://doi.org/10.1053/j.gastro.2014.01.059.
Brown, J. R.-M., Flemer, B., Joyce, S. A., Zulquernain, A., Sheehan, D., Shanahan, F., and O'Toole, P. W. (2018). Changes in microbiota composition, bile and fatty acid metabolism, in successful faecal microbiota transplantation for Clostridioides difficile infection. BMC Gastroenterol. 18, 131. https://doi.org/10.1186/s12876-018-0860-5.
Chen, E. Z., and Li, H. (2016). A two-part mixed-effects model for analyzing longitudinal microbiome compositional data. Bioinformatics 32, 2611-2617. https://doi.org/10.1093/bioinformatics/btw308.
Craven, L. J., Nair Parvathy, S., Tat-Ko, J., Burton, J. P., and Silverman, M. S. (2017). Extended Screening Costs Associated With Selecting Donors for Fecal Microbiota Transplantation for Treatment of Metabolic Syndrome-Associated Diseases. Open Forum Infect. Dis. 4, ofx243. https://doi.org/10.1093/ofid/ofx243.
DeFilipp, Z., Bloom, P. P., Torres Soto, M., Mansour, M. K., Sater, M. R. A., Huntley, M. H., Turbett, S., Chung, R. T., Chen, Y.-B., and Hohmann, E. L. (2019). Drug-Resistant E. coli Bacteremia Transmitted by Fecal Microbiota Transplant. N. Engl. J. Med. 381, 2043-2050. https://doi.org/10.1056/NEJMoa1910437.
Deshpande, A., Pasupuleti, V., Thota, P., Pant, C., Rolston, D. D. K., Hernandez, A. V., Donskey, C. J., and Fraser, T. G. (2015). Risk factors for recurrent Clostridium difficile infection: a systematic review and meta-analysis. Infect. Control Hosp. Epidemiol. 36, 452-460. https://doi.org/10.1017/ice.2014.88.
Dsouza, M., Menon, R., Crossette, E., Bhattarai, S. K., Schneider, J., Kim, Y.-G., Reddy, S., Caballero, S., Felix, C., Cornacchione, L., et al. (2022). Colonization of the live biotherapeutic product VE303 and modulation of the microbiota and metabolites in healthy volunteers. Cell Host Microbe 30, 583-598.e8. https://doi.org/10.1016/j.chom.2022.03.016.
Fachi, J. L., Felipe, J. de S., Pral, L. P., da Silva, B. K., Corrêa, R. O., de Andrade, M. C. P., da Fonseca, D. M., Basso, P. J., Câmara, N. O. S., de Sales E Souza, É. L., et al. (2019). Butyrate Protects Mice from Clostridium difficile-Induced Colitis through an HIF-1-Dependent Mechanism. Cell Rep. 27, 750-761.e7. https://doi.org/10.1016/j.celrep.2019.03.054.
Foley, M. H., O'Flaherty, S., Barrangou, R., and Theriot, C. M. (2019). Bile salt hydrolases: Gatekeepers of bile acid metabolism and host-microbiome crosstalk in the gastrointestinal tract. PLoS Pathog. 15, e1007581. https://doi.org/10.1371/journal.ppat.1007581.
Garey, K. W., Sethi, S., Yadav, Y., and DuPont, H. L. (2008). Meta-analysis to assess risk factors for recurrent Clostridium difficile infection. J. Hosp. Infect. 70, 298-304. https://doi.org/10.1016/j.jhin.2008.08.012.
Harrison, X. A., Donaldson, L., Correa-Cano, M. E., Evans, J., Fisher, D. N., Goodwin, C. E. D., Robinson, B. S., Hodgson, D. J., and Inger, R. (2018). A brief introduction to mixed effects modelling and multi-model inference in ecology. PeerJ 6, e4794. https://doi.org/10.7717/peerj.4794.
Henn, M. R., O'Brien, E. J., Diao, L., Feagan, B. G., Sandborn, W. J., Huttenhower, C., Wortman, J. R., McGovern, B. H., Wang-Weigand, S., Lichter, D. I., et al. (2021). A Phase 1b Safety Study of SER-287, a Spore-Based Microbiome Therapeutic, for Active Mild to Moderate Ulcerative Colitis. Gastroenterology 160, 115-127.e30. https://doi.org/10.1053/j.gastro.2020.07.048.
Kang, J. D., Myers, C. J., Harris, S. C., Kakiyama, G., Lee, I.-K., Yun, B.-S., Matsuzaki, K., Furukawa, M., Min, H.-K., Bajaj, J. S., et al. (2019). Bile Acid 7α-Dehydroxylating Gut Bacteria Secrete Antibiotics that Inhibit Clostridium difficile: Role of Secondary Bile Acids. Cell Chem. Biol. 26, 27-34.e4. https://doi.org/10.1016/j.chembiol.2018.10.003.
Khanna, S., Montassier, E., Schmidt, B., Patel, R., Knights, D., Pardi, D. S., and Kashyap, P. (2016). Gut microbiome predictors of treatment response and recurrence in primary Clostridium difficile infection. Aliment. Pharmacol. Ther. 44, 715-727. https://doi.org/10.1111/apt.13750.
Khanna, S., Pardi, D. S., Jones, C., Shannon, W. D., Gonzalez, C., and Blount, K. (2021). RBX7455, a Non-frozen, Orally Administered Investigational Live Biotherapeutic, Is Safe, Effective, and Shifts Patients' Microbiomes in a Phase 1 Study for Recurrent Clostridioides difficile Infections. Clin. Infect. Dis. 73, e1613-e1620. https://doi.org/10.1093/cid/ciaa1430.
Larcombe, S., Hutton, M. L., Riley, T. V., Abud, H. E., and Lyras, D. (2018). Diverse bacterial species contribute to antibiotic-associated diarrhoea and gastrointestinal damage. J. Infect. 77, 417-426. https://doi.org/10.1016/j.jinf.2018.06.006.
Mallick, H., Rahnavard, A., McIver, L. J., Ma, S., Zhang, Y., Nguyen, L. H., Tickle, T. L., Weingart, G., Ren, B., Schwager, E. H., et al. (2021). Multivariable association discovery in population-scale meta-omics studies. PLoS Comput. Biol. 17, e1009442. https://doi.org/10.1371/journal.pcbi.1009442.
Martinez, E., Taminiau, B., Rodriguez, C., and Daube, G. (2022). Gut Microbiota Composition Associated with Clostridioides difficile Colonization and Infection. Pathogens 11. https://doi.org/10.3390/pathogens11070781.
McDonald, L. C., Gerding, D. N., Johnson, S., Bakken, J. S., Carroll, K. C., Coffin, S. E., Dubberke, E. R., Garey, K. W., Gould, C. V., Kelly, C., et al. (2018). Clinical Practice Guidelines for Clostridium difficile Infection in Adults and Children: 2017 Update by the Infectious Diseases Society of America (IDSA) and Society for Healthcare Epidemiology of America (SHEA). Clin. Infect. Dis. 66, e1-e48. https://doi.org/10.1093/cid/cix1085.
Minot, S. S., Krumm, N., and Greenfield, N. B. (2015). One Codex: A Sensitive and Accurate Data Platform for Genomic Microbial Identification. BioRxiv https://doi.org/10.1101/027607.
Motamedi, H., Fathollahi, M., Abiri, R., Kadivarian, S., Rostamian, M., and Alvandi, A. (2021). A worldwide systematic review and meta-analysis of bacteria related to antibiotic-associated diarrhea in hospitalized patients. PLoS ONE 16, e0260667. https://doi.org/10.1371/journal.pone.0260667.
Murphy, J. I., Weaver, N. E., and Hendricks, A. E. (2022). Accessible analysis of longitudinal data with linear mixed effects models. Dis. Model. Mech. 15. https://doi.org/10.1242/dmm.048025.
Murphy, J., Weaver, N. E., and Hendricks, A. E. (2020). Accessible Analysis of Longitudinal Data with Linear Mixed Effects Models. BioRxiv https://doi.org/10.1101/2020.12.03.411058.
Palleja, A., Mikkelsen, K. H., Forslund, S. K., Kashani, A., Allin, K. H., Nielsen, T., Hansen, T. H., Liang, S., Feng, Q., Zhang, C., et al. (2018). Recovery of gut microbiota of healthy adults following antibiotic exposure. Nat. Microbiol. 3, 1255-1265. https://doi.org/10.1038/s41564-018-0257-9.
Palmieri, L.-J., Rainteau, D., Sokol, H., Beaugerie, L., Dior, M., Coffin, B., Humbert, L., Eguether, T., Bado, A., Hoys, S., et al. (2018). Inhibitory Effect of Ursodeoxycholic Acid on Clostridium difficile Germination Is Insufficient to Prevent Colitis: A Study in Hamsters and Humans. Front. Microbiol. 9, 2849. https://doi.org/10.3389/fmicb.2018.02849.
Rybak, M., Lomaestro, B., Rotschafer, J. C., Moellering, R., Craig, W., Billeter, M., Dalovisio, J. R., and Levine, D. P. (2009). Therapeutic monitoring of vancomycin in adult patients: a consensus review of the American Society of Health-System Pharmacists, the Infectious Diseases Society of America, and the Society of Infectious Diseases Pharmacists. Am. J. Health Syst. Pharm. 66, 82-98. https://doi.org/10.2146/ajhp080434.
Seekatz, A. M., Theriot, C. M., Rao, K., Chang, Y.-M., Freeman, A. E., Kao, J. Y., and Young, V. B. (2018). Restoration of short chain fatty acid and bile acid metabolism following fecal microbiota transplantation in patients with recurrent Clostridium difficile infection. Anaerobe 53, 64-73. https://doi.org/10.1016/j.anaerobe.2018.04.001.
Song, J. H., and Kim, Y. S. (2019). Recurrent Clostridium difficile Infection: Risk Factors, Treatment, and Prevention. Gut Liver 13, 16-24. https://doi.org/10.5009/gnl18071.
Sorg, J. A., and Sonenshein, A. L. (2008). Bile salts and glycine as cogerminants for Clostridium difficile spores. J. Bacteriol. 190, 2505-2512. https://doi.org/10.1128/JB.01765-07.
Thanissery, R., Winston, J. A., and Theriot, C. M. (2017). Inhibition of spore germination, growth, and toxin activity of clinically relevant C. difficile strains by gut microbiota derived secondary bile acids. Anaerobe 45, 86-100. https://doi.org/10.1016/j.anaerobe.2017.03.004.
Theriot, C. M., Koenigsknecht, M. J., Carlson, P. E., Hatton, G. E., Nelson, A. M., Li, B., Huffnagle, G. B., Z Li, J., and Young, V. B. (2014). Antibiotic-induced shifts in the mouse gut microbiome and metabolome increase susceptibility to Clostridium difficile infection. Nat. Commun. 5, 3114. https://doi.org/10.1038/ncomms4114.
Therneau, T. M. (2021). Survival Analysis [R package survival version 3.2-12].
Therneau, T. M., and Grambsch, P. M. (2000). The Cox Model. In Modeling survival data: extending the cox model (New York, NY: Springer New York), pp. 39-77.
Weingarden, A. R., Chen, C., Bobr, A., Yao, D., Lu, Y., Nelson, V. M., Sadowsky, M. J., and Khoruts, A. (2014). Microbiota transplantation restores normal fecal bile acid composition in recurrent Clostridium difficile infection. Am. J. Physiol. Gastrointest. Liver Physiol. 306, G310-9. https://doi.org/10.1152/ajpgi.00282.2013.
Weingarden, A. R., Chen, C., Zhang, N., Graiziger, C. T., Dosa, P. I., Steer, C. J., Shaughnessy, M. K., Johnson, J. R., Sadowsky, M. J., and Khoruts, A. (2016). Ursodeoxycholic Acid Inhibits Clostridium difficile Spore Germination and Vegetative Growth, and Prevents the Recurrence of Ileal Pouchitis Associated With the Infection. J. Clin. Gastroenterol. 50, 624-630. https://doi.org/10.1097/MCG.0000000000000427.
Winston, J. A., and Theriot, C. M. (2019). Diversification of host bile acids by members of the gut microbiota. Gut Microbes 1-14. https://doi.org/10.1080/19490976.2019.1674124.
Yun, B., Song, M., Park, D.-J., and Oh, S. (2017). Beneficial Effect of Bifidobacterium longum ATCC 15707 on Survival Rate of Clostridium difficile Infection in Mice. Korean Journal for Food Science of Animal Resources 37, 368-375. https://doi.org/10.5851/kosfa.2017.37.3.368.
Zollner-Schwetz, I., Högenauer, C., Joainig, M., Weberhofer, P., Gorkiewicz, G., Valentin, T., Hinterleitner, T. A., and Krause, R. (2008). Role of Klebsiella oxytoca in antibiotic-associated diarrhea. Clin. Infect. Dis. 47, e74-8. https://doi.org/10.1086/592074.

What is claimed is:

1. A method for treating or preventing recurrent Clostridioides difficile infection (rCDI) in a subject, the method comprising administering to the subject:
(a) a bacterial strain belonging to the species Clostridium bolteae;
(b) a bacterial strain belonging to the species Anaerotruncus colihominis; and/or
(c) a bacterial strain belonging to the species Flavonifractor plautii;
wherein at least one of the bacterial strains is detected in a fecal sample obtained from the subject at least two weeks after administration of the bacterial strains.

2. The method of claim 1, wherein at least one of the bacterial strains is detected in a fecal sample obtained from the subject at least 3, 4, 5, 6, 7, 8, 9, 10, 11, 12, 13, 14, 15, 16, 17, 18, 19, 20, 21, 22, 23, or 24 weeks after administration of the bacterial strains.

3. The method of claim 1 or 2, wherein the method comprises administering:
(a) the bacterial strain belonging to the species Clostridium bolteae;
(b) the bacterial strain belonging to the species Anaerotruncus colihominis;
(c) the bacterial strain belonging to the species Flavonifractor plautii;
(d) a bacterial strain belonging to the species Sellimonas intestinalis;
(e) a bacterial strain belonging to the species Clostridium symbiosum;
(f) a bacterial strain belonging to the species Blautia producta;
(g) a bacterial strain belonging to the species Dorea longicatena; and
(h) a bacterial strain belonging to the species Clostridium innocuum.

4. The method of any one of claims 1-3, wherein the bacterial strain belonging to the species Flavonifractor plautii is detected in a fecal sample obtained from the subject at least two weeks after administration of the bacterial strains.

5. The method of any one of claims 1-4, wherein the method further comprises measuring the abundance of the bacterial strains belonging to species Clostridium bolteae, Anaerotruncus colihominis, and/or Flavonifractor plautii in a fecal sample obtained from the subject at least two weeks after administration of the bacterial strains.

6. The method of any one of claims 1-5, wherein the method further comprises:
(i) identifying a bacterial strain belonging to the species Clostridium bolteae, Anaerotruncus colihominis, or Flavonifractor plautii as not having colonized the subject, if the bacterial strain is not detected in the fecal sample; and
(ii) administering to the subject one or more additional doses of the bacterial strain that did not colonize the subject.

7. A method for treating or preventing recurrent Clostridioides difficile infection (rCDI) in a subject, the method comprising administering to the subject:
(a) a bacterial strain comprising a 16S rDNA sequence with at least 97% sequence identity to the nucleotide sequence of SEQ ID NO: 1;
(b) a bacterial strain comprising a 16S rDNA sequence with at least 97% sequence identity to the nucleotide sequence of SEQ ID NO: 2; and/or
(c) a bacterial strain comprising a 16S rDNA sequence with at least 97% sequence identity to the nucleotide sequence of SEQ ID NO: 8;
wherein at least one of the bacterial strains is detected in a fecal sample obtained from the subject at least two weeks after administration of the bacterial strains.

8. The method of claim 7, wherein:
(a) the bacterial strain of (a) comprises a 16S rDNA sequence with at least 99% sequence identity to the nucleotide sequence of SEQ ID NO: 1;
(b) the bacterial strain of (b) comprises a 16S rDNA sequence with at least 99% sequence identity to the nucleotide sequence of SEQ ID NO: 2; and
(c) the bacterial strain of (c) comprises a 16S rDNA sequence with at least 99% sequence identity to the nucleotide sequence of SEQ ID NO: 8.

9. The method of claim 7 or 8, wherein at least one of the bacterial strains is detected in a fecal sample obtained from the subject at least 3, 4, 5, 6, 7, 8, 9, 10, 11, 12, 13, 14, 15, 16, 17, 18, 19, 20, 21, 22, 23, or 24 weeks after administration of the bacterial strains.

10. The method of claim 7 or 9, wherein the method comprises administering:
(a) a bacterial strain comprising a 16S rDNA sequence with at least 97% sequence identity to the nucleotide sequence of SEQ ID NO: 1;
(b) a bacterial strain comprising a 16S rDNA sequence with at least 97% sequence identity to the nucleotide sequence of SEQ ID NO: 2;
(c) a bacterial strain comprising a 16S rDNA sequence with at least 97% sequence identity to the nucleotide sequence of SEQ ID NO: 8;
(d) a bacterial strain comprising a 16S rDNA sequence with at least 97% sequence identity to the nucleotide sequence of SEQ ID NO: 3;
(e) a bacterial strain comprising a 16S rDNA sequence with at least 97% sequence identity to the nucleotide sequence of SEQ ID NO: 4;
(f) a bacterial strain comprising a 16S rDNA sequence with at least 97% sequence identity to the nucleotide sequence of SEQ ID NO: 5;
(g) a bacterial strain comprising a 16S rDNA sequence with at least 97% sequence identity to the nucleotide sequence of SEQ ID NO: 6; and
(h) a bacterial strain comprising a 16S rDNA sequence with at least 97% sequence identity to the nucleotide sequence of SEQ ID NO: 7.

11. The method of claim 10, wherein:
(a) the bacterial strain of (a) comprises a 16S rDNA sequence with at least 99% sequence identity to the nucleotide sequence of SEQ ID NO: 1;
(b) the bacterial strain of (b) comprises a 16S rDNA sequence with at least 99% sequence identity to the nucleotide sequence of SEQ ID NO: 2;
(c) the bacterial strain of (c) comprises a 16S rDNA sequence with at least 99% sequence identity to the nucleotide sequence of SEQ ID NO: 8;
(d) the bacterial strain of (d) comprises a 16S rDNA sequence with at least 99% sequence identity to the nucleotide sequence of SEQ ID NO: 3;
(e) the bacterial strain of (e) comprises a 16S rDNA sequence with at least 99% sequence identity to the nucleotide sequence of SEQ ID NO: 4;
(f) the bacterial strain of (f) comprises a 16S rDNA sequence with at least 99% sequence identity to the nucleotide sequence of SEQ ID NO: 5;
(g) the bacterial strain of (g) comprises a 16S rDNA sequence with at least 99% sequence identity to the nucleotide sequence of SEQ ID NO: 6; and
(h) the bacterial strain of (h) comprises a 16S rDNA sequence with at least 99% sequence identity to the nucleotide sequence of SEQ ID NO: 7.

12. The method of any one of claims 7-11, wherein the bacterial strain of (c) is detected in a fecal sample obtained from the subject at least two weeks after administration of the bacterial strains.

13. The method of any one of claims 7-12, wherein the method further comprises measuring the abundance of the bacterial strain of (a), (b), and/or (c) in a fecal sample obtained from the subject at least two weeks after administration of the bacterial strains.

14. The method of any one of claims 7-13, wherein the method further comprises:
(i) identifying the bacterial strain of (a), (b), and/or (c) as not having colonized the subject, if the bacterial strain is not detected in the fecal sample; and
(ii) administering to the subject one or more additional doses of the bacterial strain that did not colonize the subject.

15. The method of any one of claims 1-14, wherein the bacterial strains are comprised in a pharmaceutical composition comprising the bacterial strains and a pharmaceutically acceptable excipient.

16. The method of claim 15, wherein the pharmaceutical composition is administered orally or rectally.

17. The method of claim 15, wherein the pharmaceutical composition is administered orally.

18. The method of any one of claims 15-17, wherein the pharmaceutical composition is formulated for delivery to the intestine.

19. The method of any one of claims 15-18, wherein the pharmaceutical composition is formulated for delivery to the colon.

20. The method of any one of claims 15-19, wherein the pharmaceutical composition comprises one or more enteric polymers.

21. The method of any one of claims 15-20, wherein the pharmaceutical composition is administered to the subject as multiple doses over multiple days.

22. The method of claim 21, wherein the subject is administered about 1.6×10⁹ colony-forming units (CFUs) of the bacterial strains per day.

23. The method of any one of claims 15-22, wherein the subject is administered a total of about 2.2×10¹⁰ colony-forming units (CFUs) of the bacterial strains.

24. The method of claim 21, wherein the subject is administered about 8.0×10⁹ colony-forming units (CFUs) of the bacterial strains per day.

25. The method of any one of claim 15-22, or 24, wherein the subject is administered about 1.1×10¹¹ colony-forming units (CFUs) of the bacterial strains.

26. The method of any one of claims 15-25, wherein the method comprises administering an antibiotic to the subject prior to administration of the pharmaceutical composition.

27. The method of claim 26, wherein the antibiotic is vancomycin.

28. The method of any one of claims 1-27, wherein the subject is a human.


SEQUENCE LISTING (10 sequences)

SEQ ID NO: 1 - DNA, 1530 bp, Clostridium bolteae
  source 1..1530: mol_type = genomic DNA
atgagagtttgatcctggctcaggatgaacgctggcggcgtgcctaacacatgcaagtcgaacgaagcaattaaaatgaagttttcggatggatttttgattgactgagtggcggacgggtgagtaacgcgtggataacctgcctcacactgggggataacagttagaaatgactgctaataccgcataagcgcacagtaccgcatggtacggtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatcctcttgaccggcgtgtaacggcgccttcccttcggggcaggagagacaggtggtgcatggttgtcgtcagctcgtgtcgtgagatgttgggttaagtcccgcaacgagcgcaacccttatccttagtagccagcaggtagagctgggcactctagggagactgccagggataacctggaggaaggtggggatgacgtcaaatcatcatgccccttatgatttgggctacacacgtgctacaatggcgtaaacaaagggaagcaagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaactcgcaagagagggagctgccgaaggcggggcaggtaactggggtgaagtcgtaacaaggtagccgtatcggaaggtgcggctggatcacctccttt

SEQ ID NO: 2 - DNA, 1521 bp, Anaerotruncus colihominis
  source 1..1521: mol_type = genomic DNA
caaagagtttgatcctggctcaggacgaacgctggcggcgcgcctaacacatgcaagtcgaacggagcttacgttttgaagttttcggatggatgaatgtaagcttagtggcggacgggtgagtaacacgtgagcaacctgcctttcagagggggataacagccggaaacggctgctaataccgcatgatgttgcgggggcacatgcccctgcaaccaaaggagcaatccgctgaaagatgggctcgcgtccgattagccagttggcggggtaacggcccaccaaagcgacgatcggtagccggactgagaggttgaacggccacattgggactgagacacggcccagactcctacgggaggcagcagtgggggatattgcacaatgggcgaaagcctgatgcagcgacgccgcgtgagggaagacggtcttcggattgtaaacctctgtctttggggaagaaaatgacggtacccaaagaggaagctccggctaactacgtgccagcagccgcggtaatacgtagggagcaagcgttgtccggaattactgggtgtaaagggagcgtaggcgggatggcaagtagaatgttaaatccatcggctcaaccggtggctgcgttctaaactgccgttcttgagtgaagtagaggcaggcggaattcctagtgtagcggtgaaatgcgtagatattaggaggaacaccagtggcgaaggcggcctgctgggctttaactgacgctgaggctcgaaagcgtggggagcaaacaggattagataccctggtagtccacgccgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatcggatgcatagcctagagataggtgaagcccttcggggcatccagacaggtggtgcatggttgtcgtcagctcgtgtcgtgagatgttgggttaagtcccgcaacgagcgcaacccttattattagttgctacgcaagagcactctaatgagactgccgttgacaaaacggaggaaggtggggatgacgtcaaatcatcatgccccttatgacctgggctacacacgtactacaatggcactaaaacagagggcggcgacaccgcgaggtgaagcgaatcccgaaaaagtgtctcagttcagattgcaggctgcaacccgcctgcatgaagtcggaattgctagtaatcgcggatcagcatgccgcggtgaatacgttcccgggccttgtacacaccgcccgtcacaccatgggagtcggtaacacccgaagccagtagcctaaccgcaaggggggcgctgtcgaaggtgggattgatgactggggtgaagtcgtaacaaggtagccgtatcggaaggtgcggctggatcacctccttt

SEQ ID NO: 3 - DNA, 1528 bp, unidentified
  source 1..1528: mol_type = genomic DNA; note = Sellimonas intestinalis
acgagagtttgatcctggctcaggatgaacgctggcggcgtgcctaacacatgcaagtcgagcgaagcgctgttttcagaatcttcggaggaagaggacagtgactgagcggcggacgggtgagtaacgcgtgggcaacctgcctcatacagggggataacagttagaaatgactgctaataccgcataagcgcacaggaccgcatggtgtagtgtgaaaaactccggtggtatgagatggacccgcgtctgattaggtagttggtggggtaaaggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgaaggaagaagtatttcggtatgtaaacttctatcagcagggaagaagatgacggtacctgagtaagaagcaccggctaaatacgtgccagcagccgcggtaatacgtatggtgcaagcgttatccggatttactgggtgtaaagggagcgtagacggataggcaagtctggagtgaaaacccagggctcaaccctgggactgctttggaaactgcagatctggagtgccggagaggtaagcggaattcctagtgtagcggtgaaatgcgtagatattaggaggaacaccagtggcgaaggcggcttactggacggtgactgacgttgaggctcgaaagcgtggggagcaaacaggattagataccctggtagtccacgccgtaaacgatgactactaggtgtcggtgtgcaaagcacatcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctggtcttgacatccggatgacgggcgagtaatgtcgccgtcccttcggggcatccgagacaggtggtgcatggttgtcgtcagctcgtgtcgtgagatgttgggttaagtcccgcaacgagcgcaacccttatcttcagtagccagcatataaggtgggcactctggagagactgccagggagaacctggaggaaggtggggatgacgtcaaatcatcatgccccttatggccagggctacacacgtgctacaatggcgtaaacaaagggaagcgagagggtgacctggagcgaatcccaaaaataacgtctcagttcggattgtagtctgcaactcgactacatgaagctggaatcgctagtaatcgcggatcagcatgccgcggtgaatacgttcccgggtcttgtacacaccgcccgtcacaccatgggagtcagtaacgcccgaagccagtgacccaaccttagaggagggagctgtcgaaggcgggacggataactggggtgaagtcgtaacaaggtagccgtatcggaaggtgcggctggatcacctccttt

SEQ ID NO: 4 - DNA, 1527 bp, Clostridium symbiosum
  source 1..1527: mol_type = genomic DNA
atgagagtttgatcctggctcaggatgaacgctggcggcgtgcctaacacatgcaagtcgaacgaagcgatttaacggaagttttcggatggaagttggattgactgagtggcggacgggtgagtaacgcgtgggtaacctgccttgtactgggggacaacagttagaaatgactgctaataccgcataagcgcacagtatcgcatgatacagtgtgaaaaactccggtggtacaagatggacccgcgtctgattagctagttggtaaggtaacggcttaccaaggcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggtaaagcaagtctgaagtgaaagcccgcggctcaactgcgggactgctttggaaactgtttaactggagtgtcggagaggtaagtggaattcctagtgtagcggtgaaatgcgtagatattaggaggaacaccagtggcgaaggcgacttactggacgataactgacgttgaggctcgaaagcgtggggagcaaacaggattagataccctggtagtccacgccgtaaacgatgaatactaggtgttggggagcaaagctcttcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaggtcttgacatcgatccgacgggggagtaacgtccccttcccttcggggcggagaagacaggtggtgcatggttgtcgtcagctcgtgtcgtgagatgttgggttaagtcccgcaacgagcgcaacccttattctaagtagccagcggttcggccgggaactcttgggagactgccagggataacctggaggaaggtggggatgacgtcaaatcatcatgccccttatgatctgggctacacacgtgctacaatggcgtaaacaaagagaagcaagaccgcgaggtggagcaaatctcaaaaataacgtctcagttcggactgcaggctgcaactcgcctgcacgaagctggaatcgctagtaatcgcgaatcagaatgtcgcggtgaatacgttcccgggtcttgtacacaccgcccgtcacaccatgggagtcagtaacgcccgaagtcagtgacccaaccgcaaggagggagctgccgaaggcgggaccgataactggggtgaagtcgtaacaaggtagccgtatcggaaggtgcggctggatcacctccttt

SEQ ID NO: 5 - DNA, 1530 bp, Blautia producta
  source 1..1530: mol_type = genomic DNA
tcagagagtttgatcctggctcaggatgaacgctggcggcgtgcttaacacatgcaagtcgagcgaagcacttaagtggatctcttcggattgaaacttatttgactgagcggcggacgggtgagtaacgcgtgggtaacctgcctcatacagggggataacagttagaaatggctgctaataccgcataagcgcacaggaccgcatggtctggtgtgaaaaactccggtggtatgagatggacccgcgtctgattagctagttggaggggtaacggcccaccaaggcgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgtttttctagagtgccggagaggtaagcggaattcctagtgtagcggtgaaatgcgtagatattaggaggaacatcagtggcgaaggcggcttactggacggtaactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgaccggcccgtaacggggccttcccttcggggcagaggagacaggtggtgcatggttgtcgtcagctcgtgtcgtgagatgttgggttaagtcccgcaacgagcgcaacccctatccttagtagccagcaggtgaagctgggcactctagggagactgccggggataacccggaggaaggcggggacgacgtcaaatcatcatgccccttatgatttgggctacacacgtgctacaatggcgtaaacaaagggaagcgagacagcgatgttgagcaaatcccaaaaataacgtcctagttcggactgcagtctgcaactcgactgcacgaagctggaatcgctagtaatcgcgaatcagaatgtcgcggtgaatacgttcccgggtcttgtacacaccgcccgtcacaccatgggagtcagtaacgcccgaagtcagtgacccaaccttacaggagggagctgccgaaggcgggaccgataactggggtgaagtcgtaacaaggtagccgtatcggaaggtgcggctggatcacctccttt

SEQ ID NO: 6 - DNA, 1529 bp, Dorea longicatena
  source 1..1529: mol_type = genomic DNA
aacgagagtttgatcctggctcaggatgaacgctggcggcgtgcttaacacatgcaagtcgagcgaagcactttggaaagattcttcggatgatttcctttgtgactgagcggcggacgggtgagtaacgcgtgggtaacctgcctcatacagggggataacagttagaaatgactgctaataccgcataagaccacggtaccgcatggtacagtggtaaaaactccggtggtatgagatggacccgcgtctgattaggtagttggtggggtaacggcctaccaagccgacgatcagtagccgacctgagagggtgaccggccacattgggactgagacacggcccagactcctacgggaggcagcagtggggaatattgcacaatggaggaaactctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcacggcaagccagatgtgaaagcccggggctcaaccccgggactgcatttggaactgctgagctagagtgtcggagaggcaagtggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactgctaggtgtcgggtggcaaagccattcggtgccgcagctaacgcaataagcagtccacctggggagtacgttcgcaagaatgaaactcaaaggaattgacggggacccgcacaagcggtggagcatgtggtttaattcgaagcaacgcgaagaaccttacctgatcttgacatcccgatgaccgcttcgtaatggaagcttttcttcggaacatcggtgacaggtggtgcatggttgtcgtcagctcgtgtcgtgagatgttgggttaagtcccgcaacgagcgcaacccctatcttcagtagccagcaggttaagctgggcactctggagagactgccagggataacctggaggaaggtggggatgacgtcaaatcatcatgccccttatgaccagggctacacacgtgctacaatggcgtaaacaaagagaagcgaactcgcgagggtaagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcagtaacgcccgaagtcagtgacccaaccgtaaggagggagctgccgaaggtgggaccgataactggggtgaagtcgtaacaaggtagccgtatcggaaggtgcggctggatcacctccttt

SEQ ID NO: 7 - DNA, 1537 bp, Clostridium innocuum
  source 1..1537: mol_type = genomic DNA
atggagagtttgatcctggctcaggatgaacgctggcggcatgcctaatacatgcaagtcgaacgaagtttcgaggaagcttgcttccaaagagacttagtggcgaacgggtgagtaacacgtaggtaacctgcccatgtgtccgggataactgctggaaacggtagctaaaaccggataggtatacagagcgcatgctcagtatattaaagcgcccatcaaggcgtgaacatggatggacctgcggcgcattagctagttggtgaggtaacggcccaccaaggcgatgatgcgtagccggcctgagagggtaaacggccacattgggactgagacacggcccaaactcctacgggaggcagcagtagggaattttcgtcaatgggggaaaccctgaacgagcaatgccgcgtgagtgaagaaggtcttcggatcgtaaagctctgttgtaagtgaagaacggctcatagaggaaatgctatgggagtgacggtagcttaccagaaagccacggctaactacgtgccagcagccgcggtaatacgtaggtggcaagcgttatccggaatcattgggcgtaaagggtgcgtaggtggcgtactaagtctgtagtaaaaggcaatggctcaaccattgtaagctatggaaactggtatgctggagtgcagaagagggcgatggaattccatgtgtagcggtaaaatgcgtagatatatggaggaacaccagtggcgaaggcggtcgcctggtctgtaactgacactgaggcacgaaagcgtggggagcaaataggattagataccctagtagtccacgccgtaaacgatgagaactaagtgttggaggaattcagtgctgcagttaacgcaataagttctccgcctggggagtatgcacgcaagtgtgaaactcaaaggaattgacgggggcccgcacaagcggtggagtatgtggtttaattcgaagcaacgcgaagaaccttaccaggccttgacatggaaacaaataccctagagatagggggataattatggatcacacaggtggtgcatggttgtcgtcagctcgtgtcgtgagatgttgggttaagtcccgcaacgagcgcaacccttgtcgcatgttaccagcatcaagttggggactcatgcgagactgccggtgacaaaccggaggaaggtggggatgacgtcaaatcatcatgccccttatggcctgggctacacacgtactacaatggcgaccacaaagagcagcgacacagtgatgtgaagcgaatctcataaaggtcgtctcagttcggattgaagtctgcaactcgacttcatgaagtcggaatcgctagtaatcgcagatcagcatgctgcggtgaatacgttctcgggccttgtacacaccgcccgtcaaaccatgggagtcagtaatacccgaagccggtggcataaccgcaaggagtgagccgtcgaaggtaggaccgatgactggggttaagtcgtaacaaggtatccctacgggaacgtggggatggatcacctccttt

SEQ ID NO: 8 - DNA, 1530 bp, Flavonifractor plautii
  source 1..1530: mol_type = genomic DNA
tattgagagtttgatcctggctcaggatgaacgctggcggcgtgcttaacacatgcaagtcgaacggggtgctcatgacggaggattcgtccaacggattgagttacctagtggcggacgggtgagtaacgcgtgaggaacctgccttggagaggggaataacactccgaaaggagtgctaataccgcatgatgcagttgggtcgcatggctctgactgccaaagatttatcgctctgagatggcctcgcgtctgattagctagtaggcggggtaacggcccacctaggcgacgatcagtagccggactgagaggttgaccggccacattgggactgagacacggcccagactcctacgggaggcagcagtggggaatattgggcaatgggcgcaagcctgacccagcaacgccgcgtgaaggaagaaggctttcgggttgtaaacttcttttgtcggggacgaaacaaatgacggtacccgacgaataagccacggctaactacgtgccagcagccgcggtaatacgtaggtggcaagcgttatccggatttactgggtgtaaagggcgtgtaggcgggattgcaagtcagatgtgaaaactgggggctcaacctccagcctgcatttgaaactgtagttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacaggattagataccctggtagtccacgccgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcccactaacgaagcagagatgcattaggtgcccttcggggaaagtggagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggtggttaacagagggaggcaataccgcgaggtggagcaaatccctaaaagccatcccagttcggattgcaggctgaaacccgcctgtatgaagttggaatcgctagtaatcgcggatcagcatgccgcggtgaatacgttcccgggccttgtacacaccgcccgtcacaccatgagagtcgggaacacccgaagtccgtagcctaaccgcaaggagggcgcggccgaaggtgggttcgataattggggtgaagtcgtaacaaggtagccgtatcggaaggtgcggctggatcacctccttt

SEQ ID NO: 9 - DNA, 1530 bp, unidentified; identical to SEQ ID NO: 8
  source 1..1530: mol_type = genomic DNA; note = Lachnospiraceae bacterium

SEQ ID NO: 10 - DNA, 1525 bp, Bifidobacterium longum
  source 1..1525: mol_type = genomic DNA
tttgtggagggttcgattctggctcaggatgaacgctggcggcgtgcttaacacatgcaagtcgaacgggatccatcaagcttgcttggtggtgagagtggcgaacgggtgagtaatgcgtgaccgacctgccccatacaccggaatagctcctggaaacgggtggtaatgccggatgttccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggtcgtagagatacggcttcccttcggggcgggttcacaggtggtgcatggtcgtcgtcagctcgtgtcgtgagatgttgggttaagtcccgcaacgagcgcaaccctcgccccgtgttgccagcggattatgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaaggtgaggctcgtgattgggactaagtcgtaacaaggtagccgtaccggaaggtgcggctggatcacctccttt